(19) United States
(12) Patent Application Publication
(10) Pub. No.: US 2024/0408086 A1
(43) Pub. Date: Dec. 12, 2024

(54) USE OF LUVADAXISTAT FOR THE TREATMENT OF COGNITIVE IMPAIRMENT

(71) Applicants: TAKEDA PHARMACEUTICAL COMPANY LIMITED, Osaka (JP); NEUROCRINE BIOSCIENCES, INC., San Diego, CA (US)

(72) Inventors: Naga Venkatesha Murthy PATHI JAGANNATHAM, Cambridge, MA (US); Brian HAREL, Cambridge, MA (US); Nicholas DEMARTINIS, Cambridge, MA (US); Eduardo DUNAYEVICH, Cambridge, MA (US); Patricio O’DONNELL, Cambridge, MA (US); Thomas MACEK, Lexington, MA (US); Satjit Singh BRAR, San Diego, CA (US); Maura FUREY, San Diego, CA (US); Tingting GE, San Diego, CA (US); Jaskaran SINGH, San Diego, CA (US)

(21) Appl. No.: 18/548,469

(22) PCT Filed: Feb. 28, 2022
(86) PCT No.: PCT/US2022/018112

§ 371 Date: Aug. 30, 2023

Related U.S. Application Data
(60) Provisional application No. 63/200,327, filed on Mar. 1, 2021.
(60) Provisional application No. 63/229,945, filed on Aug. 5, 2021.
(60) Provisional application No. 63/264,747, filed on Dec. 1, 2021.
(60) Provisional application No. 63/265,628, filed on Dec. 17, 2021.

(51) Int. Cl. A61K 31/50; A61K 9/20; A61K 9/28; A61K 45/06; A61P 25/18; A61P 25/28

(52) U.S. Cl. CPC A61K 31/50; A61K 9/2013; A61K 9/2018; A61K 9/2054; A61K 9/2813; A61K 9/2866; A61K 45/06; A61P 25/18; A61P 25/28

(57) ABSTRACT

Methods of treating at least one cognitive symptom, e.g., at least one cognitive symptom associated with schizophrenia, comprising administering at least one compound chosen from Compound (I) and pharmaceutically acceptable salts thereof are disclosed. Also disclosed are methods of increasing D-serine levels, synaptic plasticity, and/or long-term potentiation comprising administering at least one compound chosen from Compound (I) and pharmaceutically acceptable salts thereof.

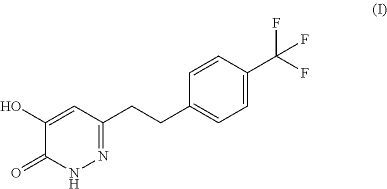 [SMILES: O=c1[nH]nc(CCc2ccc(C(F)(F)F)cc2)cc1O]

This application claims the benefit of priority of U.S. Provisional Application No. 63/200,327, filed Mar. 1, 2021; U.S. Provisional Application No. 63/229,945, filed Aug. 5, 2021; U.S. Provisional Application No. 63/264,747, filed Dec. 1, 2021; U.S. Provisional Application No. 63/265,628, filed Dec. 17, 2021, the contents of each of which are incorporated by reference herein in their entirety.

Disclosed herein are methods of treating cognitive impairment in a patient in need thereof. Methods of treating at least one cognitive symptom, e.g., at least one cognitive symptom associated with schizophrenia, in a patient in need thereof, as well as methods of increasing synaptic plasticity and/or long-term potentiation in a patient in need thereof, are also disclosed.

Schizophrenia is a severe mental disorder that affects approximately 1% of the population, with lifetime prevalence estimates ranging from 5.6 to 11.9 per 1000 persons. Schizophrenia is characterized by psychosis, cognitive impairments, and/or social and motivational deficits. For example, schizophrenia may be characterized by positive symptoms (e.g., hallucinations or delusions), negative symptoms (e.g., anhedonia, avolition, blunted affect, reduced spontaneous speech, and social withdrawal), and/or cognitive impairment associated with schizophrenia (CIAS). Cognitive symptoms of schizophrenia affect a wide range of domains, including, but not limited to, attention, working memory, and/or executive functions. While positive symptoms of schizophrenia tend to relapse and remit, in today's environment, negative and cognitive symptoms of schizophrenia are often chronic and impact social functioning for those afflicted, reflecting limited current knowledge on the course of symptom progression and available treatments.

Although negative and cognitive symptoms are highly predictive of quality of life and functional recovery, there are no approved treatments for the cognitive impairment associated with schizophrenia. Accordingly, there is a need for novel treatments of cognitive impairment, including cognitive impairment associated with schizophrenia.

D-amino acid oxidase (DAAO) is a peroxisomal enzyme that degrades neutral D-amino acids such as D-serine, a N-methyl-D-aspartate (NMDA) receptor co-agonist. Along with glutamate, D-serine mediates NMDA receptor transmission, synaptic plasticity, and other physiological functions. Additionally, D-serine is an endogenous ligand for the delta (δ)2 glutamate receptor (GluRδ2), which has been implicated in synaptic plasticity and long-term depression.

Thus, DAAO inhibitors may be useful for treating cognitive impairment, including treating cognitive symptoms associated with schizophrenia and other psychiatric disorders (e.g., psychotic disorders) and neurological disorders.

Compound (I) is a DAAO inhibitor of the following structure:

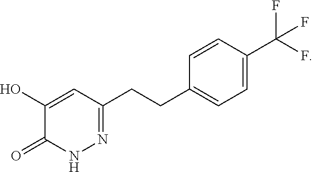 [SMILES: O=c1[nH]nc(CCc2ccc(C(F)(F)F)cc2)cc1O]

See PCT Publication No. WO 2013/027000, which is incorporated herein by reference, e.g., Example 36. Compound (I) is also referred to as luvadaxistat, TAK-831, and NBI-1065844.

Disclosed herein is a method of treating cognitive impairment associated with schizophrenia in a patient in need thereof comprising administering to the patient 1 mg to 500 mg (e.g., 1 mg to 100 mg) of at least one compound chosen from Compound (I) and pharmaceutically acceptable salts thereof once daily, wherein at least one cognitive symptom/domain associated with schizophrenia in the patient is treated by the administration.

Also disclosed herein is at least one compound chosen from Compound (I) and pharmaceutically acceptable salts thereof for use in a method of treating cognitive impairment associated with schizophrenia in a patient in need thereof, the method comprising administering to the patient 1 mg to 500 mg (e.g., 1 mg to 100 mg) of the at least one compound chosen from Compound (I) and pharmaceutically acceptable salts thereof once daily, wherein at least one cognitive symptom associated with schizophrenia in the patient is treated by the administration.

Also disclosed herein is use of at least one compound chosen from Compound (I) and pharmaceutically acceptable salts thereof in the manufacture of a medicament for use in a method of treating cognitive impairment associated with schizophrenia in a patient in need thereof, the method comprising administering to the patient 1 mg to 500 mg (e.g., 1 mg to 100 mg) of the at least one compound chosen from Compound (I) and pharmaceutically acceptable salts thereof once daily, wherein at least one cognitive symptom associated with schizophrenia in the patient is treated by the administration.

In some embodiments, the method comprises:
administering to the patient 1 mg to 500 mg (e.g., 1 mg to 100 mg; 10 mg to 50 mg; 20 mg to 50 mg; 25 mg to 50 mg; 20 mg or 50 mg; 20 mg; 50 mg) of at least one compound chosen from Compound (I) and pharmaceutically acceptable salts thereof once daily for 6 to 8 days;
determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
continuing to administer to the patient 1 mg to 500 mg (e.g., 1 mg to 100 mg; 20 mg to 50 mg; 20 mg or 50 mg; 20 mg; 50 mg) of at least one compound chosen from Compound (I) and pharmaceutically acceptable salts thereof once daily if the 6 to 8 day administration improved at least one biomarker.

In some embodiments, the at least one cognitive symptom associated with schizophrenia is chosen from impaired verbal memory, impaired working memory, impaired motor function, impaired attention, impaired processing speed, impaired verbal fluency, and impaired executive function. In some embodiments, the at least one cognitive symptom associated with schizophrenia is chosen from impaired attention, impaired memory, impaired reasoning, impaired problem solving, impaired working memory, impaired processing speed, impaired language functions, and impaired social cognition.

In some embodiments, the administration improves the patient's eye-blink conditioning (EBC) response. In some embodiments, the administration improves the patient's mismatch negativity (MMN) amplitude. In some embodiments, the administration improves the patient's auditory steady state response (ASSR) gamma band power.

In some embodiments, the administration increases the patient's D-serine levels.

In some embodiments, the administration improves the patient's performance on a cognitive measure. In some embodiments, the administration improves the patient's performance on a measure of cognitive domains. In some embodiments, the administration improves the patient's total score on cognitive measures.

In some embodiments, the administration improves the patient's Brief Assessment of Cognition in Schizophrenia (BACS) Composite Score relative to a BACS Composite Score measured for the patient prior to the administration. In some embodiments, the administration improves the patient's Schizophrenia Cognition Rating Scale (SCORS) interviewer total score relative to a SCORS interviewer total score measured for the patient prior to the administration. In some embodiments, the administration improves the patient's performance on the Continuous Performance Test-Identical Pairs (CPT-IP) test relative to a CPT-IP test prior to the administration. In some embodiments, the administration improves the patient's performance on the Brief Visuospatial Memory Test-Revised (BVMT-R) test relative to the patient's performance on a BVMT-R test prior to the administration. In some embodiments, the administration improves the patient's performance on the Mayer-Salovey-Caruso Emotional Intelligence Test (MSCEIT) relative to the patient's performance on a MSCEIT prior to the administration. In some embodiments, the administration improves the patient's performance on the Virtual Reality Functional Capacity Assessment Tool (VRFCAT) relative to the patient's performance on a VRFCAT prior to the administration. In some embodiments, the administration improves the patient's Clinical Global Impression-Severity Scale (CGI-S) score relative to a CGI-S score measured for the patient prior to the administration.

In some embodiments, the patient has at least one stable negative symptom associated with schizophrenia prior to the administration. In some embodiments, the at least one stable negative symptom associated with schizophrenia is stable for at least one month prior to the administration. In some embodiments, the at least one stable negative symptom associated with schizophrenia is chosen from anhedonia, loss of motivation, and reduced interest in social interaction. In some embodiments, the stability of the at least one stable negative symptom associated with schizophrenia is assessed/measured using the Positive and Negative Syndrome Scale (PANSS). In some embodiments, the stability of the at least one stable negative symptom associated with schizophrenia is assessed using the Brief Negative Symptom Scale (BNSS) instrument.

In some embodiments, the patient does not exhibit any depressive or extrapyramidal symptoms prior to the administration. In some embodiments, the patient was administered a stable antipsychotic treatment at a 2-6 mg daily dose of risperidone equivalents prior to the administration.

In some embodiments, there is not statistically significant improvement in any negative symptom associated with schizophrenia in the patient following the administration.

In some embodiments, the administration does not treat at least one negative symptom associated with schizophrenia. In some embodiments, the at least one negative symptom associated with schizophrenia is measured according to Positive and Negative Syndrome Scale (PANSS).

In some embodiments, the at least one compound is administered in combination with at least one additional active agent. In some embodiments, the at least one additional active agent treats at least one negative symptom associated with schizophrenia in the patient. In some embodiments, the at least one negative symptom associated with schizophrenia is chosen from anhedonia, loss of motivation, and reduced interest in social interaction.

In some embodiments, the at least one additional therapeutic agent is a hypnotic. In some embodiments, the at least one additional therapeutic agent is chosen from aripiprazole, olanzapine, risperidone, and quetiapine fumarate.

In some embodiments, the at least one compound is administered to the patient for more than 14 weeks. In some embodiments, the at least one compound is administered to the patient for more than 20 weeks.

In some embodiments, the patient is administered 20 mg to 50 mg of the at least one compound once daily. In some embodiments, the patient is administered 20 mg of the at least one compound once daily. In some embodiments, the patient is administered 50 mg of the at least one compound once daily.

In some embodiments, the patient is administered 20 mg of the at least one compound once daily and then the patient is administered 50 mg of the at least one compound once daily. In some embodiments, the patient is administered 20 mg of the at least one compound once daily for 14 weeks and then the patient is administered 50 mg of the at least one compound once daily. In some embodiments, the patient is administered 20 mg of the at least one compound once daily for 14 weeks and then the patient is administered 50 mg of the at least one compound once daily for 54 weeks.

In some embodiments, the at least one compound is orally administered. In some embodiments, the at least one compound is administered in the form of at least one tablet. In some embodiments, the at least one compound is administered in the form of at least one film-coated tablet. In some embodiments, the at least one compound is administered in the form of two film-coated tablets.

In some embodiments, the at least one compound is administered in the morning. In some embodiments, the at least one compound is administered with water or milk.

In some embodiments, the patient does not consume juice within 1 hour before or 1 hour after the administration.

In some embodiments, the patient was diagnosed with schizophrenia at least one year prior to the administration. In some embodiments, the patient was diagnosed with schizophrenia as defined by the Diagnostic and Statistical Manual of Mental Disorders (DSM-5). In some embodiments, the patient was diagnosed with schizophrenia as defined by the MINI Version 7.0.2.

In some embodiments, the patient is on a stable regimen of psychotropic medications. In some embodiments, the patient is on a regimen of psychotropic medications, wherein the psychotropic medication doses have not increased for at least 2 months prior to the administration. In some embodiments, the patient is on a regimen of psychotropic medications, wherein the psychotropic medication doses have not decreased by more than 25% for at least 2 months prior to the administration.

In some embodiments, the patient has stable symptomatology for at least 3 months prior to the administration.

In some embodiments, the patient was diagnosed with schizophrenia after the age of 12 years old. In some embodiments, the patient has not received a lifetime diagnosis of schizoaffective disorder, a lifetime diagnosis of bipolar disorder, or a lifetime diagnosis of obsessive-compulsive disorder. In some embodiments, the patient does not suffer from depression prior the administration. In some embodiments, the patient does not suffer from depression as measured by a Calgary Depression Scale for Schizophrenia Score (CDSS) prior the administration.

Also disclosed herein is a method of treating at least one cognitive symptom associated with schizophrenia in a patient, wherein the patient has no more than moderate-severe positive symptoms associated with schizophrenia (<5 rating on PANSS positive symptom items P1, P3, P4, P5, P6), comprising administering to the patient 1 mg to 500 mg (e.g., 1 mg to 100 mg) of at least one compound chosen from Compound (I) and pharmaceutically acceptable salts thereof once daily, wherein at least one cognitive symptom associated with schizophrenia in the patient is treated by the administration.

Also disclosed herein is at least one compound chosen from Compound (I) and pharmaceutically acceptable salts thereof for use in a method of treating at least one cognitive symptom associated with schizophrenia in a patient, wherein the patient has no more than moderate-severe positive symptoms associated with schizophrenia (<5 rating on PANSS positive symptom items P1 (delusions), P3 (hallucinatory behavior), P4 (excitement), P5 (grandiosity), and/or P6 (suspiciousness), the method comprising administering to the patient 1 mg to 500 mg (e.g., 1 mg to 100 mg) of the at least one compound chosen from Compound (I) and pharmaceutically acceptable salts thereof once daily.

Also disclosed herein is use of at least one compound chosen from Compound (I) and pharmaceutically acceptable salts thereof in the manufacture of a medicament for use in a method of treating at least one cognitive symptom associated with schizophrenia in a patient, wherein the patient has no more than moderate-severe positive symptoms associated with schizophrenia (<5 rating on PANSS positive symptom items P1, P3, P4, P5, P6), the method comprising administering to the patient 1 mg to 500 mg (e.g., 1 mg to 100 mg) of the at least one compound chosen from Compound (I) and pharmaceutically acceptable salts thereof once daily.

In some embodiments, the method comprises:
administering to the patient 1 mg to 500 mg (e.g., 1 mg to 100 mg; 10 mg to 50 mg; 20 mg to 50 mg; 25 mg to 50 mg; 20 mg or 50 mg; 20 mg; 50 mg) of at least one compound chosen from Compound (I) and pharmaceutically acceptable salts thereof once daily for 6 to 8 days;
determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
continuing to administer to the patient 1 mg to 500 mg (e.g., 1 mg to 100 mg; 10 mg to 50 mg; 20 mg to 50 mg; 25 mg to 50 mg; 20 mg or 50 mg; 20 mg; 50 mg) of at least one compound chosen from Compound (I) and pharmaceutically acceptable salts thereof once daily if the 6 to 8 day administration improved at least one biomarker.

In some embodiments, the administration improves the patient's eye-blink conditioning (EBC) response. In some embodiments, the administration improves the patient's mismatch negativity (MMN) amplitude. In some embodiments, the administration improves the patient's auditory steady state response (ASSR) gamma band power.

In some embodiments, the administration increases the patient's D-serine levels.

In some embodiments, the administration improves the patient's performance on a cognitive measure. In some embodiments, the administration improves the patient's performance on a measure of cognitive domains. In some embodiments, the administration improves the patient's total score on cognitive measures.

In some embodiments, the administration improves the patient's Brief Assessment of Cognition in Schizophrenia (BACS) Composite Score relative to a BACS Composite Score measured for the patient prior to the administration. In some embodiments, the administration improves the patient's Schizophrenia Cognition Rating Scale (SCORS) interviewer total score relative to a SCORS interviewer total score measured for the patient prior to the administration. In some embodiments, the administration improves the patient's performance on the Continuous Performance Test-Identical Pairs (CPT-IP) test relative to a CPT-IP test prior to the administration. In some embodiments, the administration improves the patient's performance on the Brief Visuospatial Memory Test-Revised (BVMT-R) test relative to the patient's performance on a BVMT-R test prior to the administration. In some embodiments, the administration improves the patient's performance on the Mayer-Salovey-Caruso Emotional Intelligence Test (MSCEIT) relative to the patient's performance on a MSCEIT prior to the administration. In some embodiments, the administration improves the patient's performance on the Virtual Reality Functional Capacity Assessment Tool (VRFCAT) relative to the patient's performance on a VRFCAT prior to the administration. In some embodiments, the administration improves the patient's Clinical Global Impression-Severity Scale (CGI-S) score relative to a CGI-S score measured for the patient prior to the administration.

In some embodiments, there is not statistically significant improvement in any negative symptom associated with schizophrenia in the patient following the administration.

In some embodiments, the administration does not treat at least one negative symptom associated with schizophrenia.

In some embodiments, the patient does not exhibit any depressive or extrapyramidal symptoms prior to the administration. In some embodiments, the patient was administered a stable antipsychotic treatment at a 2-6 mg daily dose of risperidone equivalents prior to the administration.

In some embodiments, the at least one compound is administered to the patient for more than 14 weeks. In some embodiments, the at least one compound is administered to the patient for more than 20 weeks.

In some embodiments, the patient is administered 20 mg to 50 mg of the at least one compound once daily. In some embodiments, the patient is administered 20 mg of the at least one compound once daily. In some embodiments, the patient is administered 50 mg of the at least one compound once daily.

In some embodiments, the patient is administered 20 mg of the at least one compound once daily and then the patient is administered 50 mg of the at least one compound once daily. In some embodiments, the patient is administered 20 mg of the at least one compound once daily for 14 weeks and then the patient is administered 50 mg of the at least one compound once daily. In some embodiments, the patient is administered 20 mg of the at least one compound once daily for 14 weeks and then the patient is administered 50 mg of the at least one compound once daily for 54 weeks.

In some embodiments, the at least one compound is administered orally. In some embodiments, the at least one compound is administered in the form of at least one tablet. In some embodiments, the at least one compound is administered in the form of at least one film-coated tablet. In some embodiments, the at least one compound is administered in the form of two film-coated tablets.

In some embodiments, the at least one compound is administered in the morning. In some embodiments, the at least one compound is administered with water or milk.

In some embodiments, the patient does not consume juice within 1 hour before or 1 hour after the administration.

In some embodiments, the at least one compound is administered in combination with at least one additional active agent. In some embodiments, the at least one additional active agent treats at least one negative symptom associated with schizophrenia in the patient. In some embodiments, the at least one additional active agent treats behavioral problems associated with at least one negative symptom of schizophrenia in the patient. In some embodiments, the severity of the at least one symptom is measured according to Positive and Negative Syndrome Scale (PANSS).

In some embodiments, the at least one negative symptom associated with schizophrenia is a motivational syndrome. In some embodiments, the at least one negative symptom associated with schizophrenia is chosen from anhedonia, affective flattening, loss of motivation, diminished energy, social withdrawal, and reduced interest in social interaction.

In some embodiments, the at least one additional therapeutic agent is a hypnotic. In some embodiments, the at least one additional therapeutic agent is chosen from aripiprazole, olanzapine, risperidone, and quetiapine fumarate.

In some embodiments, the at least one compound is administered in combination with at least one additional active agent. In some embodiments, the at least one additional active agent treats at least one negative symptom associated with schizophrenia in the patient. In some embodiments, the at least one negative symptom associated with schizophrenia is chosen from anhedonia, loss of motivation, and reduced interest in social interaction.

In some embodiments, the patient was diagnosed with schizophrenia at least one year prior to the administration. In some embodiments, the patient was diagnosed with schizophrenia as defined by the Diagnostic and Statistical Manual of Mental Disorders (DSM-5). In some embodiments, the patient was diagnosed with schizophrenia as defined by the MINI Version 7.0.2.

In some embodiments, the patient is on a stable regimen of psychotropic medications. In some embodiments, the patient is on a regimen of psychotropic medications, wherein the psychotropic medication doses have not increased for at least 2 months prior to the administration. In some embodiments, the patient is on a regimen of psychotropic medications, wherein the psychotropic medication doses have not decreased by more than 25% for at least 2 months prior to the administration.

In some embodiments, the patient has stable symptomatology for at least 3 months prior to the administration.

In some embodiments, the patient was diagnosed with schizophrenia after the age of 12 years old. In some embodiments, the patient has not received a lifetime diagnosis of schizoaffective disorder, a lifetime diagnosis of bipolar disorder, or a lifetime diagnosis of obsessive-compulsive disorder. In some embodiments, the patient does not suffer from depression prior the administration. In some embodiments, the patient does not suffer from depression as measured by a Calgary Depression Scale for Schizophrenia Score (CDSS) prior the administration.

Also disclosed herein is a method of treating cognitive impairment associated with schizophrenia in a patient in need thereof comprising administering to the patient 50 mg to 500 mg of at least one compound chosen from Compound (I) and pharmaceutically acceptable salts thereof once daily, wherein at least one cognitive symptom/domain associated with schizophrenia in the patient is treated by the administration. In some embodiments, the at least one cognitive symptom/domain associated with schizophrenia is expressed by a broad range of cognitive dysfunction(s). In some embodiments, the at least one cognitive symptom/domain associated with schizophrenia is poor information processing, impaired ability to focus on objectives, abnormalities of working memory and learning, or any combination thereof.

Also disclosed herein is at least one compound chosen from Compound (I) and pharmaceutically acceptable salts thereof for use in a method of treating cognitive impairment associated with schizophrenia in a patient in need thereof, the method comprising administering to the patient 50 mg to 500 mg of the at least one compound chosen from Compound (I) and pharmaceutically acceptable salts thereof once daily, wherein at least one cognitive symptom/domain associated with schizophrenia in the patient is treated by the administration.

Also disclosed herein is use of at least one compound chosen from Compound (I) and pharmaceutically acceptable salts thereof in the manufacture of a medicament for use in a method of treating cognitive impairment associated with schizophrenia in a patient in need thereof, the method comprising administering to the patient 50 mg to 500 mg of the at least one compound chosen from Compound (I) and pharmaceutically acceptable salts thereof once daily, wherein at least one cognitive symptom/domain associated with schizophrenia in the patient is treated by the administration.

In some embodiments, the method comprises:
administering to the patient 50 mg to 500 mg of at least one compound chosen from Compound (I) and pharmaceutically acceptable salts thereof once daily for 6 to 8 days;
determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
continuing to administer to the patient 50 mg to 500 mg of at least one compound chosen from Compound (I) and pharmaceutically acceptable salts thereof once daily if the 6 to 8 day administration improved at least one biomarker.

In some embodiments, the at least one cognitive symptom associated with schizophrenia is chosen from impaired verbal memory, impaired working memory, impaired motor function, impaired attention, impaired processing speed, impaired verbal fluency, and impaired executive function. In some embodiments, the at least one cognitive symptom associated with schizophrenia is chosen from impaired attention, impaired memory, impaired reasoning, impaired problem solving, impaired working memory, impaired processing speed, impaired language functions, and impaired social cognition.

In some embodiments, the administration improves the patient's eye-blink conditioning (EBC) response. In some embodiments, the administration improves the patient's mismatch negativity (MMN) amplitude. In some embodiments, the administration improves the patient's auditory steady state response (ASSR) gamma band power.

In some embodiments, the administration increases the patient's D-serine levels.

In some embodiments, the administration improves the patient's performance on a cognitive measure. In some embodiments, the administration improves the patient's performance on a measure of cognitive domains. In some embodiments, the administration improves the patient's total score on cognitive measures.

In some embodiments, the administration improves the patient's Brief Assessment of Cognition in Schizophrenia (BACS) Composite Score relative to a BACS Composite Score measured for the patient prior to the administration. In some embodiments, the administration improves the patient's Schizophrenia Cognition Rating Scale (SCORS) interviewer total score relative to a SCORS interviewer total score measured for the patient prior to the administration. In some embodiments, the administration improves the patient's performance on the Continuous Performance Test-Identical Pairs (CPT-IP) test relative to a CPT-IP test prior to the administration. In some embodiments, the administration improves the patient's performance on the Brief Visuospatial Memory Test-Revised (BVMT-R) test relative to the patient's performance on a BVMT-R test prior to the administration. In some embodiments, the administration improves the patient's performance on the Mayer-Salovey-Caruso Emotional Intelligence Test (MSCEIT) relative to the patient's performance on a MSCEIT prior to the administration. In some embodiments, the administration improves the patient's performance on the Virtual Reality Functional Capacity Assessment Tool (VRFCAT) relative to the patient's performance on a VRFCAT prior to the administration. In some embodiments, the administration improves the patient's Clinical Global Impression-Severity Scale (CGI-S) score relative to a CGI-S score measured for the patient prior to the administration.

In some embodiments, the patient has at least one stable negative symptom associated with schizophrenia prior to the administration. In some embodiments, the at least one stable negative symptom associated with schizophrenia is stable for at least one month prior to the administration. In some embodiments, the at least one stable negative symptom associated with schizophrenia is chosen from anhedonia, loss of motivation, and reduced interest in social interaction. In some embodiments, the stability of the at least one stable negative symptom associated with schizophrenia is assessed using the PANSS assessment. In some embodiments, the stability of the at least one stable negative symptom associated with schizophrenia is assessed using the Brief Negative Symptom Scale (BNSS) instrument.

In some embodiments, the patient does not exhibit any depressive or extrapyramidal symptoms prior to the administration. In some embodiments, the patient was administered a stable antipsychotic treatment at a 2-6 mg daily dose of risperidone equivalents prior to the administration.

In some embodiments, there is not statistically significant improvement in any negative symptom associated with schizophrenia in the patient following the administration.

In some embodiments, the administration does not treat at least one negative symptom associated with schizophrenia.

In some embodiments, the at least one compound is administered in combination with at least one additional active agent. In some embodiments, the at least one additional active agent treats at least one negative symptom associated with schizophrenia in the patient. In some embodiments, the at least one negative symptom associated with schizophrenia is chosen from anhedonia, loss of motivation, and reduced interest in social interaction.

In some embodiments, the at least one additional therapeutic agent is a hypnotic. In some embodiments, the at least one additional therapeutic agent is chosen from aripiprazole, olanzapine, risperidone, and quetiapine fumarate.

In some embodiments, the at least one compound is administered to the patient for more than 14 weeks. In some embodiments, the at least one compound is administered to the patient for more than 20 weeks.

In some embodiments, the patient is administered 50 mg of the at least one compound once daily. In some embodiments, the patient is administered 125 mg of the at least one compound once daily. In some embodiments, the patient is administered 500 mg of the at least one compound once daily. In some embodiments, the patient is administered less than 500 mg of the at least one compound once daily.

In some embodiments, the at least one compound is administered in the form of at least one film-coated tablet. In some embodiments, the at least one compound is administered in the form of two film-coated tablets.

In some embodiments, the at least one compound is administered in the morning. In some embodiments, the at least one compound is administered with water or milk.

In some embodiments, the patient does not consume juice within 1 hour before or 1 hour after the administration.

In some embodiments, the patient was diagnosed with schizophrenia at least one year prior to the administration. In some embodiments, the patient was diagnosed with schizophrenia as defined by the Diagnostic and Statistical Manual of Mental Disorders (DSM-5). In some embodiments, the patient was diagnosed with schizophrenia as defined by the MINI Version 7.0.2.

In some embodiments, the patient is on a stable regimen of psychotropic medications. In some embodiments, the patient is on a regimen of psychotropic medications, wherein the psychotropic medication doses have not increased for at least 2 months prior to the administration. In some embodiments, the patient is on a regimen of psychotropic medications, wherein the psychotropic medication doses have not decreased by more than 25% for at least 2 months prior to the administration.

In some embodiments, the patient has stable symptomatology for at least 3 months prior to the administration.

In some embodiments, the patient was diagnosed with schizophrenia after the age of 12 years old. In some embodiments, the patient has not received a lifetime diagnosis of schizoaffective disorder, a lifetime diagnosis of bipolar disorder, or a lifetime diagnosis of obsessive-compulsive disorder. In some embodiments, the patient does not suffer from depression prior the administration. In some embodiments, the patient does not suffer from depression as measured by a Calgary Depression Scale for Schizophrenia Score (CDSS) prior the administration.

Also disclosed herein is a method of treating at least one cognitive symptom associated with schizophrenia in a patient, wherein the patient has no more than moderate-severe positive symptoms associated with schizophrenia (<5 rating on PANSS positive symptom items P1, P3, P4, P5, P6), comprising administering to the patient 50 mg to 500 mg of at least one compound chosen from Compound (I) and pharmaceutically acceptable salts thereof once daily, wherein at least one cognitive symptom associated with schizophrenia in the patient is treated by the administration.

Also disclosed herein is at least one compound chosen from Compound (I) and pharmaceutically acceptable salts thereof for use in a method of treating at least one cognitive symptom associated with schizophrenia in a patient, wherein the patient has no more than moderate-severe positive symptoms associated with schizophrenia (<5 rating on PANSS positive symptom items P1, P3, P4, P5, P6), the method comprising administering to the patient 50 mg to 500 mg of the at least one compound chosen from Compound (I) and pharmaceutically acceptable salts thereof once daily.

Also disclosed herein is use of at least one compound chosen from Compound (I) and pharmaceutically acceptable salts thereof in the manufacture of a medicament for use in a method of treating at least one cognitive symptom associated with schizophrenia in a patient, wherein the patient has no more than moderate-severe positive symptoms associated with schizophrenia (<5 rating on PANSS positive symptom items P1, P3, P4, P5, P6), the method comprising administering to the patient 50 mg to 500 mg of the at least one compound chosen from Compound (I) and pharmaceutically acceptable salts thereof once daily.

In some embodiments, the method comprises:
administering to the patient 50 mg to 500 mg of at least one compound chosen from Compound (I) and pharmaceutically acceptable salts thereof once daily for 6 to 8 days;
determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
continuing to administer to the patient 50 mg to 500 mg of at least one compound chosen from Compound (I) and pharmaceutically acceptable salts thereof once daily if the 6 to 8 day administration improved at least one biomarker.

In some embodiments, the administration improves the patient's eye-blink conditioning (EBC) response. In some embodiments, the administration improves the patient's mismatch negativity (MMN) amplitude. In some embodiments, the administration improves the patient's auditory steady state response (ASSR) gamma band power.

In some embodiments, the administration increases the patient's D-serine levels.

In some embodiments, the administration improves the patient's performance on a cognitive measure. In some embodiments, the administration improves the patient's performance on a measure of cognitive domains. In some embodiments, the administration improves the patient's total score on cognitive measures.

In some embodiments, the administration improves the patient's Brief Assessment of Cognition in Schizophrenia (BACS) Composite Score relative to a BACS Composite Score measured for the patient prior to the administration. In some embodiments, the administration improves the patient's Schizophrenia Cognition Rating Scale (SCORS) interviewer total score relative to a SCoRS interviewer total score measured for the patient prior to the administration. In some embodiments, the administration improves the patient's performance on the Continuous Performance Test-Identical Pairs (CPT-IP) test relative to a CPT-IP test prior to the administration. In some embodiments, the administration improves the patient's performance on the Brief Visuospatial Memory Test-Revised (BVMT-R) test relative to the patient's performance on a BVMT-R test prior to the administration. In some embodiments, the administration improves the patient's performance on the Mayer-Salovey-Caruso Emotional Intelligence Test (MSCEIT) relative to the patient's performance on a MSCEIT prior to the administration. In some embodiments, the administration improves the patient's performance on the Virtual Reality Functional Capacity Assessment Tool (VRFCAT) relative to the patient's performance on a VRFCAT prior to the administration. In some embodiments, the administration improves the patient's Clinical Global Impression-Severity Scale (CGI-S) score relative to a CGI-S score measured for the patient prior to the administration.

In some embodiments, there is not statistically significant improvement in any negative symptom associated with schizophrenia in the patient following the administration.

In some embodiments, the administration does not treat at least one negative symptom associated with schizophrenia.

In some embodiments, the patient does not exhibit any depressive or extrapyramidal symptoms prior to the administration. In some embodiments, the patient was administered a stable antipsychotic treatment at a 2-6 mg daily dose of risperidone equivalents prior to the administration.

In some embodiments, the at least one compound is administered to the patient for more than 14 weeks. In some embodiments, the at least one compound is administered to the patient for more than 20 weeks.

In some embodiments, the patient is administered 50 mg of the at least one compound once daily. In some embodiments, the patient is administered 125 mg of the at least one compound once daily. In some embodiments, the patient is administered 500 mg of the at least one compound once daily. In some embodiments, the patient is administered less than 500 mg of the at least one compound once daily.

In some embodiments, the at least one compound is administered in the form of at least one film-coated tablet. In some embodiments, the at least one compound is administered in the form of two film-coated tablets.

In some embodiments, the at least one compound is administered in the morning. In some embodiments, the at least one compound is administered with water or milk.

In some embodiments, the patient does not consume juice within 1 hour before or 1 hour after the administration.

In some embodiments, the at least one compound is administered in combination with at least one additional active agent. In some embodiments, the at least one additional active agent treats at least one negative symptom associated with schizophrenia in the patient. In some embodiments, the at least one negative symptom associated with schizophrenia is chosen from anhedonia, loss of motivation, and reduced interest in social interaction.

In some embodiments, the at least one additional therapeutic agent is a hypnotic. In some embodiments, the at least one additional therapeutic agent is chosen from aripiprazole, olanzapine, risperidone, and quetiapine fumarate.

In some embodiments, the patient was diagnosed with schizophrenia at least one year prior to the administration. In some embodiments, the patient was diagnosed with schizophrenia as defined by the Diagnostic and Statistical Manual of Mental Disorders (DSM-5). In some embodiments, the patient was diagnosed with schizophrenia as defined by the MINI Version 7.0.2.

In some embodiments, the patient is on a stable regimen of psychotropic medications. In some embodiments, the patient is on a regimen of psychotropic medications, wherein the psychotropic medication doses have not increased for at least 2 months prior to the administration. In some embodiments, the patient is on a regimen of psychotropic medications, wherein the psychotropic medication doses have not decreased by more than 25% for at least 2 months prior to the administration.

In some embodiments, the patient has stable symptomatology for at least 3 months prior to the administration.

In some embodiments, the patient was diagnosed with schizophrenia after the age of 12 years old. In some embodiments, the patient has not received a lifetime diagnosis of schizoaffective disorder, a lifetime diagnosis of bipolar disorder, or a lifetime diagnosis of obsessive-compulsive disorder. In some embodiments, the patient does not suffer from depression prior the administration. In some embodiments, the patient does not suffer from depression as measured by a Calgary Depression Scale for Schizophrenia Score (CDSS) prior the administration.

Also disclosed herein is a method of treating cognitive impairment in a patient in need thereof comprising administering to the patient a therapeutically effective amount of at least one compound chosen from Compound (I) and pharmaceutically acceptable salts thereof, wherein the patient exhibits at least one cognitive symptom prior to the administration.

Also disclosed herein is at least one compound chosen from Compound (I) and pharmaceutically acceptable salts thereof for use in a method of treating cognitive impairment in a patient in need thereof, the method comprising administering to the patient a therapeutically effective amount of the at least one compound chosen from Compound (I) and pharmaceutically acceptable salts thereof, wherein the patient exhibits at least one cognitive symptom prior to the administration.

Also disclosed herein is use of at least one compound chosen from Compound (I) and pharmaceutically acceptable salts thereof in the manufacture of a medicament for use in a method of treating cognitive impairment in a patient in need thereof, the method comprising administering to the patient a therapeutically effective amount of the at least one compound chosen from Compound (I) and pharmaceutically acceptable salts thereof, wherein the patient exhibits at least one cognitive symptom prior to the administration.

In some embodiments, the method comprises:
administering to the patient a therapeutically effective amount of at least one compound chosen from Compound (I) and pharmaceutically acceptable salts thereof once daily for 6 to 8 days;
determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
continuing to administer to the patient a therapeutically effective amount of at least one compound chosen from Compound (I) and pharmaceutically acceptable salts thereof once daily if the 6 to 8 day administration improved at least one biomarker.

In some embodiments, the at least one cognitive symptom is chosen from impaired verbal memory, impaired working memory, impaired motor function, impaired attention, impaired processing speed, impaired verbal fluency, and impaired executive function. In some embodiments, the at least one cognitive symptom is chosen from impaired attention, impaired memory, impaired reasoning, impaired problem solving, impaired working memory, impaired processing speed, impaired language functions, and impaired social cognition.

In some embodiments, the administration treats the at least one cognitive symptom.

In some embodiments, the administration improves the patient's eye-blink conditioning (EBC) response. In some embodiments, the administration improves the patient's mismatch negativity (MMN) amplitude. In some embodiments, the administration improves the patient's auditory steady state response (ASSR) gamma band power.

In some embodiments, the administration increases the patient's D-serine levels.

In some embodiments, the at least one cognitive symptom is associated with a psychotic disorder. In some embodiments, the psychotic disorder is chosen from psychosis, schizophreniform disorder, schizoaffective disorder, and schizophrenia unassociated with aggression. In some embodiments, the psychotic disorder is schizophreniform disorder, schizoaffective disorder, and schizophrenia unassociated with aggression. In some embodiments, the psychotic disorder is schizophrenia unassociated with aggression.

In some embodiments, the patient suffers from dementia. In some embodiments, the patient suffers from a mood disorder.

In some embodiments, the patient suffers from a disease chosen from attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium. In some embodiments, the patient suffers from a disease chosen from Cushing's disease, Lewy body disease, multiple sclerosis, stroke, addictive disorder, pervasive development disorder, fragile X syndrome, Prader-Willi syndrome, and delirium.

In some embodiments, the patient is administered 1 mg to 100 mg of the at least one compound once daily. In some embodiments, the patient is administered 20 mg to 50 mg of the at least one compound once daily. In some embodiments, the patient is administered 20 mg of the at least one compound once daily. In some embodiments, the patient is administered 50 mg of the at least one compound once daily. In some embodiments, the patient is administered 20 mg or 50 mg of the at least one compound once daily.

In some embodiments, the patient is administered 20 mg of the at least one compound once daily and then the patient is administered 50 mg of the at least one compound once daily. In some embodiments, the patient is administered 20 mg of the at least one compound once daily for 14 weeks and then the patient is administered 50 mg of the at least one compound once daily. In some embodiments, the patient is administered 20 mg of the at least one compound once daily for 14 weeks and then the patient is administered 50 mg of the at least one compound once daily for 54 weeks.

In some embodiments, the patient is administered 50 mg to 500 mg of the at least one compound once daily. In some embodiments, the patient is administered 125 mg of the at least one compound once daily. In some embodiments, the patient is administered 500 mg of the at least one compound once daily. In some embodiments, the patient is administered less than 500 mg of the at least one compound once daily.

In some embodiments, the at least one compound is administered in the form of at least one film-coated tablet. In some embodiments, the at least one compound is administered in the form of two film-coated tablets.

In some embodiments, the at least one compound is administered in the morning. In some embodiments, the at least one compound is administered with water or milk.

In some embodiments, the patient does not consume juice within 1 hour before or 1 hour after the administration.

In some embodiments, the at least one compound is administered in combination with at least one additional active agent. In some embodiments, the at least one additional active agent treats at least one negative symptom associated with schizophrenia in the patient. In some embodiments, the at least one negative symptom associated with schizophrenia is chosen from anhedonia, loss of motivation, and reduced interest in social interaction.

Also disclosed herein is a method of increasing D-serine levels in a patient in need thereof comprising administering to the patient less than 500 mg of at least one compound chosen from Compound (I) and pharmaceutically acceptable salts thereof once daily.

Also disclosed herein is at least one compound chosen from Compound (I) and pharmaceutically acceptable salts thereof for use in a method of increasing D-serine levels in a patient in need thereof, the method comprising administering to the patient less than 500 mg of the at least one compound chosen from Compound (I) and pharmaceutically acceptable salts thereof once daily.

Also disclosed herein is use of at least one compound chosen from Compound (I) and pharmaceutically acceptable salts thereof in the manufacture of a medicament for use in a method of increasing D-serine levels in a patient in need thereof, the method comprising administering to the patient less than 500 mg of the at least one compound chosen from Compound (I) and pharmaceutically acceptable salts thereof once daily.

In some embodiments, the patient is administered 20 mg of the at least one compound once daily. In some embodiments, the patient is administered 50 mg of the at least one compound once daily. In some embodiments, the patient is administered 125 mg of the at least one compound once daily.

In some embodiments, the patient is administered 1 mg to 100 mg of the at least one compound once daily. In some embodiments, the patient is administered 20 mg to 50 mg of the at least one compound once daily.

In some embodiments, the patient is administered 20 mg or 50 mg of the at least one compound once daily.

In some embodiments, the patient is administered 20 mg of the at least one compound once daily and then the patient is administered 50 mg of the at least one compound once daily. In some embodiments, the patient is administered 20 mg of the at least one compound once daily for 14 weeks and then the patient is administered 50 mg of the at least one compound once daily. In some embodiments, the patient is administered 20 mg of the at least one compound once daily for 14 weeks and then the patient is administered 50 mg of the at least one compound once daily for 54 weeks.

In some embodiments, the patient exhibits at least one cognitive symptom prior to the administration.

In some embodiments, the at least one compound is administered to the patient for more than 14 weeks. In some embodiments, the at least one compound is administered to the patient for more than 20 weeks.

In some embodiments, the at least one compound is administered in the form of at least one film-coated tablet. In some embodiments, the at least one compound is administered in the form of two film-coated tablets.

In some embodiments, the at least one compound is administered in the morning. In some embodiments, the at least one compound is administered with water or milk.

In some embodiments, the patient does not consume juice within 1 hour before or 1 hour after the administration.

In some embodiments, the administration improves the patient's eye-blink conditioning (EBC) response. In some embodiments, the administration improves the patient's mismatch negativity (MMN) amplitude. In some embodiments, the administration improves the patient's auditory steady state response (ASSR) gamma band power.

In some embodiments, the at least one compound is administered in combination with at least one additional active agent. In some embodiments, the at least one additional therapeutic agent is a hypnotic. In some embodiments, the at least one additional therapeutic agent is chosen from aripiprazole, olanzapine, risperidone, and quetiapine fumarate.

In some embodiments, the patient suffers from a disease chosen from attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium. In some embodiments, the patient suffers from a disease chosen from Cushing's disease, Lewy body disease, multiple sclerosis, stroke, addictive disorder, pervasive development disorder, fragile X syndrome, Prader-Willi syndrome, and delirium.

In some embodiments, the patient suffers from a disease chosen from psychosis, schizophreniform disorder, schizoaffective disorder, and schizophrenia unassociated with aggression.

In some embodiments, the patient suffers from dementia. In some embodiments, the patient suffers from a mood disorder.

Also disclosed herein is a method of increasing long-term potentiation in a patient in need thereof comprising administering to the patient less than 500 mg of at least one compound chosen from Compound (I) and pharmaceutically acceptable salts thereof once daily.

Also disclosed herein is at least one compound chosen from Compound (I) and pharmaceutically acceptable salts thereof for use in a method of increasing long-term potentiation in a patient in need thereof, the method comprising administering to the patient less than 500 mg of the at least one compound chosen from Compound (I) and pharmaceutically acceptable salts thereof once daily.

Also disclosed herein is use of at least one compound chosen from Compound (I) and pharmaceutically acceptable salts thereof in the manufacture of a medicament for use in a method of increasing long-term potentiation in a patient in need thereof, the method comprising administering to the patient less than 500 mg of the at least one compound chosen from Compound (I) and pharmaceutically acceptable salts thereof once daily.

In some embodiments, the patient is administered 20 mg of the at least one compound once daily. In some embodiments, the patient is administered 50 mg of the at least one compound once daily. In some embodiments, the patient is administered 125 mg of the at least one compound once daily.

In some embodiments, the patient is administered 1 mg to 100 mg of the at least one compound once daily. In some embodiments, the patient is administered 20 mg to 50 mg of the at least one compound once daily.

In some embodiments, the patient is administered 20 mg or 50 mg of the at least one compound once daily.

In some embodiments, the patient is administered 20 mg of the at least one compound once daily and then the patient is administered 50 mg of the at least one compound once daily. In some embodiments, the patient is administered 20 mg of the at least one compound once daily for 14 weeks and then the patient is administered 50 mg of the at least one compound once daily. In some embodiments, the patient is administered 20 mg of the at least one compound once daily for 14 weeks and then the patient is administered 50 mg of the at least one compound once daily for 54 weeks.

In some embodiments, the patient exhibits at least one cognitive symptom prior to the administration.

In some embodiments, the at least one compound is administered to the patient for more than 14 weeks. In some embodiments, the at least one compound is administered to the patient for more than 20 weeks.

In some embodiments, the at least one compound is administered in the form of at least one film-coated tablet. In some embodiments, the at least one compound is administered in the form of two film-coated tablets.

In some embodiments, the at least one compound is administered in the morning. In some embodiments, the at least one compound is administered with water or milk.

In some embodiments, the patient does not consume juice within 1 hour before or 1 hour after the administration.

In some embodiments, the administration increases the patient's D-serine levels.

In some embodiments, the administration improves the patient's eye-blink conditioning (EBC) response. In some embodiments, the administration improves the patient's mismatch negativity (MMN) amplitude. In some embodiments, the administration improves the patient's auditory steady state response (ASSR) gamma band power.

In some embodiments, the at least one compound is administered in combination with at least one additional active agent. In some embodiments, the at least one additional therapeutic agent is a hypnotic. In some embodiments, the at least one additional therapeutic agent is chosen from aripiprazole, olanzapine, risperidone, and quetiapine fumarate.

Also disclosed herein is a method of increasing synaptic plasticity in a patient in need thereof comprising administering to the patient less than 500 mg of at least one compound chosen from Compound (I) and pharmaceutically acceptable salts thereof once daily.

Also disclosed herein is at least one compound chosen from Compound (I) and pharmaceutically acceptable salts thereof for use in a method of increasing synaptic plasticity in a patient in need thereof, the method comprising administering to the patient less than 500 mg of the at least one compound chosen from Compound (I) and pharmaceutically acceptable salts thereof once daily.

Also disclosed herein is use of at least one compound chosen from Compound (I) and pharmaceutically acceptable salts thereof in the manufacture of a medicament for use in a method of increasing synaptic plasticity in a patient in need thereof, the method comprising administering to the patient less than 500 mg of the at least one compound chosen from Compound (I) and pharmaceutically acceptable salts thereof once daily.

In some embodiments, the patient is administered 20 mg of the at least one compound once daily. In some embodiments, the patient is administered 50 mg of the at least one compound once daily. In some embodiments, the patient is administered 125 mg of the at least one compound once daily.

In some embodiments, the patient is administered 1 mg to 100 mg of the at least one compound once daily. In some embodiments, the patient is administered 20 mg to 50 mg of the at least one compound once daily.

In some embodiments, the patient is administered 20 mg or 50 mg of the at least one compound once daily.

In some embodiments, the patient is administered 20 mg of the at least one compound once daily and then the patient is administered 50 mg of the at least one compound once daily. In some embodiments, the patient is administered 20 mg of the at least one compound once daily for 14 weeks and then the patient is administered 50 mg of the at least one compound once daily. In some embodiments, the patient is administered 20 mg of the at least one compound once daily for 14 weeks and then the patient is administered 50 mg of the at least one compound once daily for 54 weeks.

In some embodiments, the patient exhibits at least one cognitive symptom prior to the administration.

In some embodiments, the at least one compound is administered to the patient for more than 14 weeks. In some embodiments, the at least one compound is administered to the patient for more than 20 weeks.

In some embodiments, the at least one compound is administered in the form of at least one film-coated tablet. In some embodiments, the at least one compound is administered in the form of two film-coated tablets.

In some embodiments, the at least one compound is administered in the morning. In some embodiments, the at least one compound is administered with water or milk.

In some embodiments, the patient does not consume juice within 1 hour before or 1 hour after the administration.

In some embodiments, the at least one compound is administered in the morning. In some embodiments, the at least one compound is administered with water or milk.

In some embodiments, the patient does not consume juice within 1 hour before or after the administration.

In some embodiments, the administration increases the patient's D-serine levels.

In some embodiments, the administration increases long-term potentiation in the patient.

In some embodiments, the administration improves the patient's eye-blink conditioning (EBC) response. In some embodiments, the administration improves the patient's mismatch negativity (MMN) amplitude. In some embodiments, the administration improves the patient's auditory steady state response (ASSR) gamma band power.

In some embodiments, the at least one compound is administered in combination with at least one additional active agent. In some embodiments, the at least one additional therapeutic agent is a hypnotic. In some embodiments, the at least one additional therapeutic agent is chosen from aripiprazole, olanzapine, risperidone, and quetiapine fumarate.

In some embodiments, the patient suffers from a disease chosen from attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium. In some embodiments, the patient suffers from a disease chosen from Cushing's disease, Lewy body disease, multiple sclerosis, stroke, addictive disorder, pervasive development disorder, fragile X syndrome, Prader-Willi syndrome, and delirium.

In some embodiments, the patient suffers from a disease chosen from psychosis, schizophreniform disorder, schizoaffective disorder, and schizophrenia unassociated with aggression.

In some embodiments, the patient suffers from dementia. In some embodiments, the patient suffers from a mood disorder.

It should be understood that references herein to methods of treatment (e.g., methods of treating cognitive impairment) using at least one compound chosen from Compound (I) and pharmaceutically acceptable salts should also be interpreted as references to:
at least one compound chosen from Compound (I) and pharmaceutically acceptable salts for use in methods of treating, e.g., cognitive impairment; and/or
the use of at least one compound chosen from Compound (I) and pharmaceutically acceptable salts in the manufacture of a medicament for treating, e.g., cognitive impairment.

### Non-Limiting Example Embodiments 1

Without limitation, some embodiments of the disclosure include:
1. A method of treating cognitive impairment associated with schizophrenia in a patient in need thereof comprising administering to the patient 50 mg to 500 mg of at least one compound chosen from Compound (I):

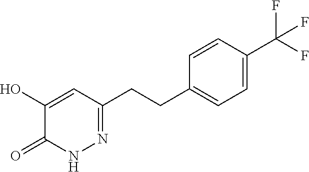 [SMILES: O=c1[nH]nc(CCc2ccc(C(F)(F)F)cc2)cc1O]

and pharmaceutically acceptable salts thereof once daily, wherein at least one cognitive symptom associated with schizophrenia in the patient is treated by the administration.
2. The method of treating cognitive impairment associated with schizophrenia according to Embodiment 1, wherein the at least one cognitive symptom associated with schizophrenia is chosen from impaired verbal memory, impaired working memory, impaired motor function, impaired attention, impaired processing speed, impaired verbal fluency, and impaired executive function.
3. The method of treating cognitive impairment associated with schizophrenia according to Embodiment 1 or 2, wherein the administration improves the patient's Brief Assessment of Cognition in Schizophrenia (BACS) Composite Score relative to a BACS Composite Score measured for the patient prior to the administration.
4. The method of treating cognitive impairment associated with schizophrenia according to any one of Embodiments 1 to 3, wherein the at least one cognitive symptom associated with schizophrenia is chosen from impaired attention, impaired memory, impaired reasoning, impaired problem solving, impaired working memory, impaired processing speed, impaired language functions, and impaired social cognition.
5. The method of treating cognitive impairment associated with schizophrenia according to any one of Embodiments 1 to 4, wherein the administration improves the patient's Schizophrenia Cognition Rating Scale (SCORS) interviewer total score relative to a SCORS interviewer total score measured for the patient prior to the administration.
6. The method of treating cognitive impairment associated with schizophrenia according to any one of Embodiments 1 to 5, wherein the patient has at least one stable negative symptom associated with schizophrenia prior to the administration.
7. The method of treating cognitive impairment associated with schizophrenia according to Embodiment 6, wherein the at least one stable negative symptom associated with schizophrenia is chosen from anhedonia, loss of motivation, and reduced interest in social interaction.
8. The method of treating cognitive impairment associated with schizophrenia according to Embodiment 6, wherein the stability of the at least one stable negative symptom associated with schizophrenia is assessed using PANSS.
9. The method of treating cognitive impairment associated with schizophrenia according to Embodiment 6 or 7, wherein the stability of the at least one stable negative symptom associated with schizophrenia is assessed using the Brief Negative Symptom Scale (BNSS) instrument.
10. The method of treating cognitive impairment associated with schizophrenia according to any one of Embodiments 6 to 9, wherein the at least one stable negative symptom associated with schizophrenia is stable for at least one month prior to the administration.
11. The method of treating cognitive impairment associated with schizophrenia according to any one of Embodiments 1 to 5, wherein there is not statistically significant improvement in any negative symptom associated with schizophrenia in the patient following the administration.
12. The method of treating cognitive impairment associated with schizophrenia according to any one of Embodiments 1 to 11, wherein the at least one compound is administered in combination with at least one additional active agent.
13. The method of treating cognitive impairment associated with schizophrenia according to Embodiment 12, wherein the at least one additional active agent treats at least one negative symptom associated with schizophrenia in the patient.
14. The method of treating cognitive impairment associated with schizophrenia according to Embodiment 13, wherein the at least one negative symptom associated with schizophrenia is chosen from anhedonia, loss of motivation, and reduced interest in social interaction.
15. The method of treating cognitive impairment associated with schizophrenia according to any one of Embodiments 1 to 14, wherein the at least one compound is administered to the patient for more than 14 weeks.
16. The method of treating cognitive impairment associated with schizophrenia according to any one of Embodiments 1 to 15, wherein the at least one compound is administered to the patient for more than 20 weeks.
17. The method of treating cognitive impairment associated with schizophrenia according to any one of Embodiments 1 to 16, wherein the patient is administered 50 mg of the at least one compound once daily.
18. The method of treating cognitive impairment associated with schizophrenia according to any one of Embodiments 1 to 16, wherein the patient is administered 125 mg of the at least one compound once daily.
19. The method of treating cognitive impairment associated with schizophrenia according to any one of Embodiments 1 to 16, wherein the patient is administered 500 mg of the at least one compound once daily.
20. A method of treating cognitive impairment in a patient in need thereof comprising administering to the patient a therapeutically effective amount of at least one compound chosen from Compound (I):

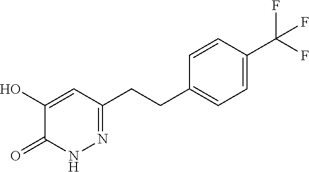 [SMILES: O=c1[nH]nc(CCc2ccc(C(F)(F)F)cc2)cc1O]

and pharmaceutically acceptable salts thereof, wherein the patient exhibits at least one cognitive symptom prior to the administration.
21. The method of treating cognitive impairment according to Embodiment 20, wherein the at least one cognitive symptom is chosen from impaired verbal memory, impaired working memory, impaired motor function, impaired attention, impaired processing speed, impaired verbal fluency, and impaired executive function.
22. The method of treating cognitive impairment according to Embodiment 20 or 21, wherein the at least one cognitive symptom is chosen from impaired attention, impaired memory, impaired reasoning, impaired problem solving, impaired working memory, impaired processing speed, impaired language functions, and impaired social cognition.
23. The method of treating cognitive impairment according to any one of Embodiments 20 to 22, wherein the administration treats the at least one cognitive symptom.
24. The method of treating cognitive impairment according to any one of Embodiments 20 to 23, wherein the at least one cognitive symptom is associated with a psychotic disorder.
25. The method of treating cognitive impairment according to Embodiment 24, wherein the psychotic disorder is chosen from psychosis, schizophreniform disorder, schizoaffective disorder, and schizophrenia unassociated with aggression.
26. The method of treating cognitive impairment according to Embodiment 24 or 25, wherein the psychotic disorder is schizophreniform disorder, schizoaffective disorder, and schizophrenia unassociated with aggression.
27. The method of treating cognitive impairment according to any one of Embodiments 24 to 26, wherein the psychotic disorder is schizophrenia unassociated with aggression.
28. The method of treating cognitive impairment according to any one of Embodiments 20 to 23, wherein the at least one cognitive symptom is associated with attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
29. The method of treating cognitive impairment according to any one of Embodiments 20 to 23, wherein the at least one cognitive symptom is associated with Cushing's disease, Lewy body disease, multiple sclerosis, stroke, addictive disorder, pervasive development disorder, fragile X syndrome, Prader-Willi syndrome, and delirium.
30. The method of treating cognitive impairment according to any one of Embodiments 20 to 29, wherein the patient is administered 50 mg to 500 mg of the at least one compound once daily.
31. The method of treating cognitive impairment according to any one of Embodiments 20 to 30, wherein the at least one compound is administered in combination with at least one additional active agent.
32. The method of treating cognitive impairment according to Embodiment 31, wherein the patient has schizophrenia and the at least one additional active agent treats at least one negative symptom in the patient.
33. The method of treating cognitive impairment according to Embodiment 32, wherein the at least one negative symptom is chosen from anhedonia, loss of motivation, and reduced interest in social interaction.
34. The method of treating cognitive impairment according to any one of Embodiments 20 to 33, wherein the patient has at least one stable negative symptom chosen from anhedonia, loss of motivation, and reduced interest in social interaction prior to the administration.
35. The method of treating cognitive impairment according to any one of Embodiments 1 to 34, wherein the at least one compound is administered in the form of at least one film-coated tablet.
36. A method of treating at least one cognitive symptom associated with schizophrenia in a patient, wherein the patient has no more than moderate-severe positive symptoms associated with schizophrenia (<5 rating on PANSS positive symptom items P1, P3, P4, P5, P6),
comprising administering to the patient 50 mg to 500 mg of at least one compound chosen from Compound (I):

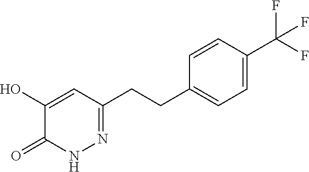 [SMILES: O=c1[nH]nc(CCc2ccc(C(F)(F)F)cc2)cc1O]

and pharmaceutically acceptable salts thereof once daily, wherein at least one cognitive symptom associated with schizophrenia in the patient is treated by the administration.
37. The method of treating at least one cognitive symptom associated with schizophrenia according to Embodiment 36, wherein the administration improves the patient's Brief Assessment of Cognition in Schizophrenia (BACS) Composite Score relative to a BACS Composite Score measured for the patient prior to the administration.
38. The method of treating at least one cognitive symptom associated with schizophrenia according to Embodiment 36 or 37, wherein the administration improves the patient's Schizophrenia Cognition Rating Scale (SCORS) interviewer total score relative to a SCORS interviewer total score measured for the patient prior to the administration.
39. The method of treating at least one cognitive symptom associated with schizophrenia according to any one of Embodiments 36 to 38, wherein there is not statistically significant improvement in any negative symptom associated with schizophrenia in the patient following the administration.
40. The method of treating at least one cognitive symptom associated with schizophrenia according to any one of Embodiments 36 to 39, wherein the administration does not treat at least one negative symptom associated with schizophrenia.
41. The method of treating at least one cognitive symptom associated with schizophrenia according to Embodiment 40, wherein the at least one negative symptom associated with schizophrenia is chosen from anhedonia, loss of motivation, and reduced interest in social interaction.
42. The method of treating cognitive impairment associated with schizophrenia according to any one of Embodiments 1 to 19, wherein the administration does not treat at least one negative symptom associated with schizophrenia.
43. The method of treating at least one cognitive symptom associated with schizophrenia according to Embodiment 42, wherein the at least one negative symptom associated with schizophrenia is chosen from anhedonia, loss of motivation, and reduced interest in social interaction.
44. A pharmaceutical composition for use in the treatment of cognitive impairment associated with schizophrenia in a patient in need thereof, wherein:
   the pharmaceutical composition comprises 50 mg to 500 mg of at least one compound chosen from Compound (I):

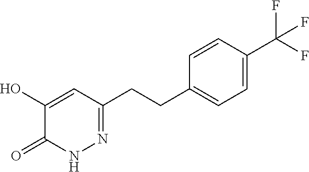 [SMILES: O=c1[nH]nc(CCc2ccc(C(F)(F)F)cc2)cc1O]

and pharmaceutically acceptable salts thereof;
   the pharmaceutical composition is for use once daily; and
   at least one cognitive symptom associated with schizophrenia in the patient is treated by the use.
45. The pharmaceutical composition for use according to Embodiment 44, wherein the at least one cognitive symptom associated with schizophrenia is chosen from impaired verbal memory, impaired working memory, impaired motor function, impaired attention, impaired processing speed, impaired verbal fluency, and impaired executive function.
46. The pharmaceutical composition for use according to Embodiment 44 or 45, wherein the use improves the patient's Brief Assessment of Cognition in Schizophrenia (BACS) Composite Score relative to a BACS Composite Score measured for the patient prior to the use.
47. The pharmaceutical composition for use according to any one of Embodiments 44 to 46, wherein the at least one cognitive symptom associated with schizophrenia is chosen from impaired attention, impaired memory, impaired reasoning, impaired problem solving, impaired working memory, impaired processing speed, impaired language functions, and impaired social cognition.
48. The pharmaceutical composition for use according to any one of Embodiments 44 to 47, wherein the use improves the patient's Schizophrenia Cognition Rating Scale (SCORS) interviewer total score relative to a SCORS interviewer total score measured for the patient prior to the use.
49. The pharmaceutical composition for use according to any one of Embodiments 44 to 48, wherein the patient has at least one stable negative symptom associated with schizophrenia prior to the use.
50. The pharmaceutical composition for use according to Embodiment 49, wherein the at least one stable negative symptom associated with schizophrenia is chosen from anhedonia, loss of motivation, and reduced interest in social interaction.
51. The pharmaceutical composition for use according to Embodiment 49, wherein the stability of the at least one stable negative symptom associated with schizophrenia is assessed using PANSS.
52. The pharmaceutical composition for use according to Embodiment 49 or 50, wherein the stability of the at least one stable negative symptom associated with schizophrenia is assessed using the Brief Negative Symptom Scale (BNSS) instrument.
53. The pharmaceutical composition for use according to any one of Embodiments 49 to 52, wherein the at least one stable negative symptom associated with schizophrenia is stable for at least one month prior to the use.
54. The pharmaceutical composition for use according to any one of Embodiments 44 to 48, wherein there is not statistically significant improvement in any negative symptom associated with schizophrenia in the patient following the use.
55. The pharmaceutical composition for use according to any one of Embodiments 44 to 54, wherein the pharmaceutical composition is for use in combination with at least one additional active agent.
56. The pharmaceutical composition for use according to Embodiment 55, wherein the at least one additional active agent treats at least one negative symptom associated with schizophrenia in the patient.
57. The pharmaceutical composition for use according to Embodiment 56, wherein the at least one negative symptom associated with schizophrenia is chosen from anhedonia, loss of motivation, and reduced interest in social interaction.
58. The pharmaceutical composition for use according to any one of Embodiments 44 to 57, wherein the pharmaceutical composition is for use for more than 14 weeks.
59. The pharmaceutical composition for use according to any one of Embodiments 44 to 58, wherein the pharmaceutical composition is for use for more than 20 weeks.
60. The pharmaceutical composition for use according to any one of Embodiments 44 to 59, wherein the pharmaceutical composition comprises 50 mg of the at least one compound.
61. The pharmaceutical composition for use according to any one of Embodiments 44 to 59, wherein the pharmaceutical composition comprises 125 mg of the at least one compound.
62. The pharmaceutical composition for use according to any one of Embodiments 44 to 59, wherein the pharmaceutical composition comprises 500 mg of the at least one compound.
63. The pharmaceutical composition for use according to any one of Embodiments 44 to 62, wherein the use does not treat a negative symptom associated with schizophrenia.
64. The pharmaceutical composition for use according to Embodiment 63, wherein the at least one negative symptom associated with schizophrenia is chosen from anhedonia, loss of motivation, and reduced interest in social interaction prior to the administration.
65. A pharmaceutical composition for use in the treatment of cognitive impairment in a patient in need thereof, wherein:
   the pharmaceutical composition comprises a therapeutically effective amount of at least one compound chosen from Compound (I):

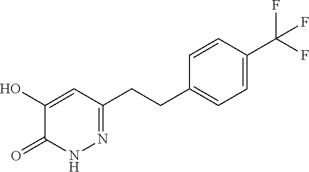 [SMILES: O=c1[nH]nc(CCc2ccc(C(F)(F)F)cc2)cc1O]

and pharmaceutically acceptable salts thereof; and
   the patient exhibits at least one cognitive symptom prior to the use.
66. The pharmaceutical composition for use according to Embodiment 65, wherein the at least one cognitive symptom associated with schizophrenia is chosen from impaired verbal memory, impaired working memory, impaired motor function, impaired attention, impaired processing speed, impaired verbal fluency, and impaired executive function.
67. The pharmaceutical composition for use according to Embodiment 65 or 66, wherein the at least one cognitive symptom is chosen from impaired attention, impaired memory, impaired reasoning, impaired problem solving, impaired working memory, impaired processing speed, impaired language functions, and impaired social cognition.
68. The pharmaceutical composition for use according to any one of Embodiments 65 to 67, wherein the use treats the at least one cognitive symptom.
69. The pharmaceutical composition for use according to any one of Embodiments 65 to 68, wherein the at least one cognitive symptom is associated with a psychotic disorder.
70. The pharmaceutical composition for use according to Embodiment 69, wherein the psychotic disorder is chosen from psychosis, schizophreniform disorder, schizoaffective disorder, and schizophrenia unassociated with aggression.
71. The pharmaceutical composition for use according to Embodiment 69 or 70, wherein the psychotic disorder is schizophreniform disorder, schizoaffective disorder, and schizophrenia unassociated with aggression.
72. The pharmaceutical composition for use according to any one of Embodiments 69 to 71, wherein the psychotic disorder is schizophrenia unassociated with aggression.
73. The pharmaceutical composition for use according to any one of Embodiments 65 to 68, wherein the at least one cognitive symptom is associated with attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
74. The pharmaceutical composition for use according to any one of Embodiments 65 to 68, wherein the at least one cognitive symptom is associated with Cushing's disease, Lewy body disease, multiple sclerosis, stroke, addictive disorder, pervasive development disorder, fragile X syndrome, Prader-Willi syndrome, and delirium.
75. The pharmaceutical composition for use according to any one of Embodiments 65 to 74, wherein:
   the pharmaceutical composition comprises 50 mg to 500 mg of the at least one compound;
   the pharmaceutical composition is for use once daily.
76. The pharmaceutical composition for use according to any one of Embodiments 65 to 75, wherein the pharmaceutical composition is for use in combination with at least one additional active agent.
77. The pharmaceutical composition for use according to Embodiment 76, wherein the patient has schizophrenia and the at least one additional active agent treats at least one negative symptom in the patient.
78. The pharmaceutical composition for use according to Embodiment 77, wherein the at least one negative symptom is chosen from anhedonia, loss of motivation, and reduced interest in social interaction.
79. The pharmaceutical composition for use according to any one of Embodiments 65 to 78, wherein the patient has at least one stable negative symptom chosen from anhedonia, loss of motivation, and reduced interest in social interaction prior to the use.
80. The pharmaceutical composition for use according to any one of Embodiments 44 to 79, wherein the pharmaceutical composition is in the form of at least one film-coated tablet.
81. A pharmaceutical composition for use in the treatment of at least one cognitive symptom associated with schizophrenia in a patient, wherein:
   the pharmaceutical composition comprises 50 mg to 500 mg of at least one compound chosen from Compound (I):

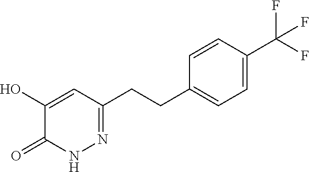 [SMILES: O=c1[nH]nc(CCc2ccc(C(F)(F)F)cc2)cc1O]

and pharmaceutically acceptable salts thereof;
   the pharmaceutical composition is for use once daily;
   the patient has no more than moderate-severe positive symptoms associated with schizophrenia (<5 rating on PANSS positive symptom items P1, P3, P4, P5, P6); and
   at least one cognitive symptom associated with schizophrenia in the patient is treated by the use.
82. The pharmaceutical composition for use according to Embodiment 81, wherein the use improves the patient's Brief Assessment of Cognition in Schizophrenia (BACS) Composite Score relative to a BACS Composite Score measured for the patient prior to the use.
83. The pharmaceutical composition for use according to Embodiment 81 or 82, wherein the use improves the patient's Schizophrenia Cognition Rating Scale (SCORS) interviewer total score relative to a SCORS interviewer total score measured for the patient prior to the use.
84. The pharmaceutical composition for use according to any one of Embodiments 81 to 83, wherein there is not statistically significant improvement in any negative symptom associated with schizophrenia in the patient following the use.
85. The pharmaceutical composition for use according to any one of Embodiments 81 to 84, wherein the use does not treat at least one negative symptom associated with schizophrenia.
86. The pharmaceutical composition for use according to Embodiment 85, wherein the at least one negative symptom associated with schizophrenia is chosen from anhedonia, loss of motivation, and reduced interest in social interaction.
87. Use of a pharmaceutical composition comprising 50 mg to 500 mg of at least one compound chosen from Compound (I):

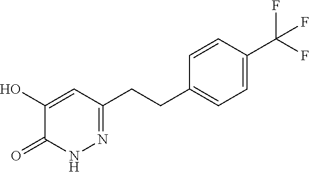 [SMILES: O=c1[nH]nc(CCc2ccc(C(F)(F)F)cc2)cc1O]

and pharmaceutically acceptable salts thereof, in the manufacture of a medicament for the treatment of cognitive impairment associated with schizophrenia in a patient in need thereof, wherein:
   the pharmaceutical composition is for use once daily; and
   at least one cognitive symptom associated with schizophrenia in the patient is treated by the use.
88. The use according to Embodiment 87, wherein the at least one cognitive symptom associated with schizophrenia is chosen from impaired verbal memory, impaired working memory, impaired motor function, impaired attention, impaired processing speed, impaired verbal fluency, and impaired executive function.
89. The use according to Embodiment 87 or 88, wherein the use improves the patient's Brief Assessment of Cognition in Schizophrenia (BACS) Composite Score relative to a BACS Composite Score measured for the patient prior to the use.
90. The use according to any one of Embodiments 87 to 89, wherein the at least one cognitive symptom associated with schizophrenia is chosen from impaired attention, impaired memory, impaired reasoning, impaired problem solving, impaired working memory, impaired processing speed, impaired language functions, and impaired social cognition.
91. The use according to any one of Embodiments 87 to 90, wherein the use improves the patient's Schizophrenia Cognition Rating Scale (SCORS) interviewer total score relative to a SCORS interviewer total score measured for the patient prior to the use.
92. The use according to any one of Embodiments 87 to 91, wherein the patient has at least one stable negative symptom associated with schizophrenia prior to the use.
93. The use according to Embodiment 92, wherein the at least one stable negative symptom associated with schizophrenia is chosen from anhedonia, loss of motivation, and reduced interest in social interaction.
94. The use according to Embodiment 92, wherein the stability of the at least one stable negative symptom associated with schizophrenia is assessed using PANSS.
95. The use according to Embodiment 92 or 93, wherein the stability of the at least one stable negative symptom associated with schizophrenia is assessed using the Brief Negative Symptom Scale (BNSS) instrument.
96. The use according to any one of Embodiments 87 to 95, wherein the at least one stable negative symptom associated with schizophrenia is stable for at least one month prior to the use.
97. The use according to any one of Embodiments 87 to 91, wherein there is not statistically significant improvement in any negative symptom associated with schizophrenia in the patient following the use.
98. The use according to any one of Embodiments 87 to 97, wherein the pharmaceutical composition is for use in combination with at least one additional active agent.
99. The use according to Embodiment 98, wherein the at least one additional active agent treats at least one negative symptom associated with schizophrenia in the patient.
100. The use according to Embodiment 99, wherein the at least one negative symptom associated with schizophrenia is chosen from anhedonia, loss of motivation, and reduced interest in social interaction.
101. The use according to any one of Embodiments 87 to 100, wherein the pharmaceutical composition is for use for more than 14 weeks.
102. The use according to any one of Embodiments 87 to 101, wherein the pharmaceutical composition is for use for more than 20 weeks.
103. The use according to any one of Embodiments 87 to 102, wherein the pharmaceutical composition comprises 50 mg of the at least one compound.
104. The use according to any one of Embodiments 87 to 102, wherein the pharmaceutical composition comprises 125 mg of the at least one compound.
105. The use according to any one of Embodiments 87 to 102, wherein the pharmaceutical composition comprises 500 mg of the at least one compound.
106. The use according to any one of Embodiments 87 to 105, wherein the use does not treat a negative symptom associated with schizophrenia.
107. The use according to Embodiment 106, wherein the at least one negative symptom associated with schizophrenia is chosen from anhedonia, loss of motivation, and reduced interest in social interaction prior to the administration.
108. Use of a pharmaceutical composition comprising a therapeutically effective amount of at least one compound chosen from Compound (I):

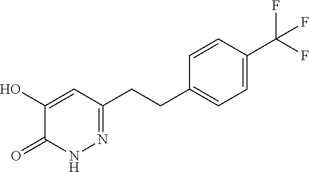 [SMILES: O=c1[nH]nc(CCc2ccc(C(F)(F)F)cc2)cc1O]

and pharmaceutically acceptable salts thereof in the treatment of cognitive impairment in a patient in need thereof, wherein the patient exhibits at least one cognitive symptom prior to the use.
109. The use according to Embodiment 108, wherein the at least one cognitive symptom is chosen from impaired verbal memory, impaired working memory, impaired motor function, impaired attention, impaired processing speed, impaired verbal fluency, and impaired executive function.
110. The use according to Embodiment 108 or 109, wherein the at least one cognitive symptom is chosen from impaired attention, impaired memory, impaired reasoning, impaired problem solving, impaired working memory, impaired processing speed, impaired language functions, and impaired social cognition.
111. The use according to any one of Embodiments 108 to 110, wherein the use treats the at least one cognitive symptom.
112. The use according to any one of Embodiments 108 to 111, wherein the at least one cognitive symptom is associated with a psychotic disorder.
113. The use according to Embodiment 112, wherein the psychotic disorder is chosen from psychosis, schizophreniform disorder, schizoaffective disorder, and schizophrenia unassociated with aggression.
114. The use according to Embodiment 112 or 113, wherein the psychotic disorder is schizophreniform disorder, schizoaffective disorder, and schizophrenia unassociated with aggression.
115. The use according to any one of Embodiments 112 to 114, wherein the psychotic disorder is schizophrenia unassociated with aggression.
116. The use according to any one of Embodiments 108 to 111, wherein the at least one cognitive symptom is associated with attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
117. The use according to any one of Embodiments 108 to 111, wherein the at least one cognitive symptom is associated with Cushing's disease, Lewy body disease, multiple sclerosis, stroke, addictive disorder, pervasive development disorder, fragile X syndrome, Prader-Willi syndrome, and delirium.
118. The use according to any one of Embodiments 108 to 117, wherein:
   the pharmaceutical composition comprises 50 mg to 500 mg of the at least one compound;
   the pharmaceutical composition is for use once daily.
119. The use according to any one of Embodiments 108 to 118, wherein the pharmaceutical composition is for use in combination with at least one additional active agent.
120. The use according to Embodiment 119, wherein the patient has schizophrenia and the at least one additional active agent treats at least one negative symptom in the patient.
121. The use according to Embodiment 120, wherein the at least one negative symptom is chosen from anhedonia, loss of motivation, and reduced interest in social interaction.
122. The use according to any one of Embodiments 108 to 121, wherein the patient has at least one stable negative symptom chosen from anhedonia, loss of motivation, and reduced interest in social interaction prior to the use.
123. The use according to any one of Embodiments 87 to 122, wherein the pharmaceutical composition is in the form of at least one film-coated tablet.
124. Use of a pharmaceutical composition comprising 50 mg to 500 mg of at least one compound chosen from Compound (I):

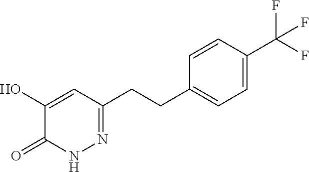 [SMILES: O=c1[nH]nc(CCc2ccc(C(F)(F)F)cc2)cc1O]

and pharmaceutically acceptable salts thereof in the treatment of at least one cognitive symptom associated with schizophrenia in a patient, wherein:
   the pharmaceutical composition is for use once daily;
   the patient has no more than moderate-severe positive symptoms associated with schizophrenia (<5 rating on PANSS positive symptom items P1, P3, P4, P5, P6); and
   at least one cognitive symptom associated with schizophrenia in the patient is treated by the use.
125. The use according to Embodiment 124, wherein the use improves the patient's Brief Assessment of Cognition in Schizophrenia (BACS) Composite Score relative to a BACS Composite Score measured for the patient prior to the use.
126. The use according to Embodiment 124 or 125, wherein the use improves the patient's Schizophrenia Cognition Rating Scale (SCORS) interviewer total score relative to a SCORS interviewer total score measured for the patient prior to the use.
127. The use according to any one of Embodiments 124 to 126, wherein there is not statistically significant improvement in any negative symptom associated with schizophrenia in the patient following the use.
128. The use according to any one of Embodiments 124 to 127, wherein the use does not treat at least one negative symptom associated with schizophrenia.
129. The use according to Embodiment 128, wherein the at least one negative symptom associated with schizophrenia is chosen from anhedonia, loss of motivation, and reduced interest in social interaction.
130. A method of increasing D-serine levels, long-term potentiation, and/or synaptic plasticity in a patient in need thereof comprising administering to the patient less than 500 mg of at least one compound chosen from Compound (I):

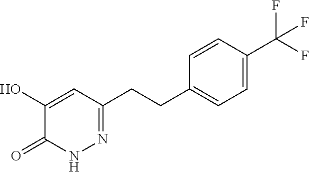 [SMILES: O=c1[nH]nc(CCc2ccc(C(F)(F)F)cc2)cc1O]

and pharmaceutically acceptable salts thereof once daily.
131. The method according to Embodiment 130, wherein the patient is administered 50 mg of the at least one compound once daily.
132. The method according to Embodiment 130 or 131, wherein the method increases D-serine levels in the patient.
133. The method according to any one of Embodiments 130 to 132, wherein the method increases long-term potentiation in the patient.
134. The method according to any one of Embodiments 130 to 133, wherein the method increases synaptic plasticity in the patient.
135. The method according to any one of Embodiments 130 to 134, wherein the patient exhibits at least one cognitive symptom prior to the administration.
136. The method according to Embodiment 135, wherein the at least one cognitive symptom is chosen from impaired verbal memory, impaired working memory, impaired motor function, impaired attention, impaired processing speed, impaired verbal fluency, and impaired executive function.
137. The method according to Embodiment 135, wherein the at least one cognitive symptom is chosen from impaired attention, impaired memory, impaired reasoning, impaired problem solving, impaired working memory, impaired processing speed, impaired language functions, and impaired social cognition.
138. The method according to any one of Embodiments 130 to 137, wherein the administration improves at least one biomarker chosen from the patient's eye-blink conditioning (EBC) response, the patient's mismatch negativity (MMN) amplitude, and the patient's auditory steady state response (ASSR) gamma band power.
139. The method according to any one of Embodiments 130 to 138, wherein the patient suffers from a disease chosen from attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
140. The method according to any one of Embodiments 130 to 138, wherein the patient suffers from a disease chosen from psychosis, schizophreniform disorder, schizoaffective disorder, and schizophrenia unassociated with aggression.
141. The method according to any one of Embodiments 130 to 138, wherein the patient has no more than moderate-severe positive symptoms associated with schizophrenia (<5 rating on PANSS positive symptom items P1, P3, P4, P5, P6).
142. A pharmaceutical composition for use in increasing D-serine levels, long-term potentiation, and/or synaptic plasticity in a patient in need thereof, wherein:
   the pharmaceutical composition comprises less than 500 mg of at least one compound chosen from Compound (I):

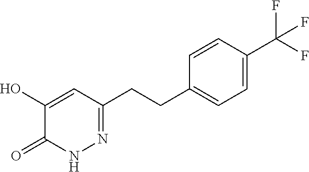 [SMILES: O=c1[nH]nc(CCc2ccc(C(F)(F)F)cc2)cc1O]

and pharmaceutically acceptable salts thereof; and
   the pharmaceutical composition is for use once daily.
143. The pharmaceutical composition for use according to Embodiment 142, wherein the pharmaceutical composition comprises 50 mg of the at least one compound.
144. The pharmaceutical composition for use according to Embodiment 142 or 143, wherein the use increases D-serine levels in the patient.
145. The pharmaceutical composition for use according to any one of Embodiments 142 to 144, wherein the use increases long-term potentiation in the patient.
146. The pharmaceutical composition for use according to any one of Embodiments 142 to 145, wherein the use increases synaptic plasticity in the patient.
147. The pharmaceutical composition for use according to any one of Embodiments 142 to 146, wherein the patient exhibits at least one cognitive symptom prior to the use.
148. The pharmaceutical composition for use according to Embodiment 147, wherein the at least one cognitive symptom is chosen from impaired verbal memory, impaired working memory, impaired motor function, impaired attention, impaired processing speed, impaired verbal fluency, and impaired executive function.
149. The pharmaceutical composition for use according to Embodiment 147, wherein the at least one cognitive symptom is chosen from impaired attention, impaired memory, impaired reasoning, impaired problem solving, impaired working memory, impaired processing speed, impaired language functions, and impaired social cognition.
150. The pharmaceutical composition for use according to any one of Embodiments 142 to 149, wherein the use improves at least one biomarker chosen from the patient's eye-blink conditioning (EBC) response, the patient's mismatch negativity (MMN) amplitude, and the patient's auditory steady state response (ASSR) gamma band power.
151. The pharmaceutical composition for use according to any one of Embodiments 142 to 150, wherein the patient suffers from a disease chosen from attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
152. The pharmaceutical composition for use according to any one of Embodiments 142 to 150, wherein the patient suffers from a disease chosen from psychosis, schizophreniform disorder, schizoaffective disorder, and schizophrenia unassociated with aggression.
153. The pharmaceutical composition for use according to any one of Embodiments 142 to 150, wherein the patient has no more than moderate-severe positive symptoms associated with schizophrenia (<5 rating on PANSS positive symptom items P1, P3, P4, P5, P6).
154. Use of a pharmaceutical composition comprising less than 500 mg of at least one compound chosen from Compound (I):

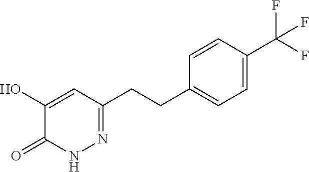 [SMILES: O=c1[nH]nc(CCc2ccc(C(F)(F)F)cc2)cc1O]

and pharmaceutically acceptable salts thereof, in the manufacture of a medicament for increasing D-serine levels, long-term potentiation, and/or synaptic plasticity in a patient in need thereof, wherein the pharmaceutical composition is for use once daily.
155. The use according to Embodiment 154, wherein the pharmaceutical composition comprises 50 mg of the at least one compound.
156. The use according to Embodiment 154 or 155, wherein the use increases D-serine levels in the patient.
157. The use according to any one of Embodiments 154 to 156, wherein the use increases long-term potentiation in the patient.
158. The use according to any one of Embodiments 154 to 157, wherein the use increases synaptic plasticity in the patient.
159. The use according to any one of Embodiments 154 to 158, wherein the patient exhibits at least one cognitive symptom prior to the use.
160. The use according to Embodiment 159, wherein the at least one cognitive symptom is chosen from impaired verbal memory, impaired working memory, impaired motor function, impaired attention, impaired processing speed, impaired verbal fluency, and impaired executive function.
161. The use according to Embodiment 159, wherein the at least one cognitive symptom is chosen from impaired attention, impaired memory, impaired reasoning, impaired problem solving, impaired working memory, impaired processing speed, impaired language functions, and impaired social cognition.
162. The use according to any one of Embodiments 154 to 161, wherein the use improves at least one biomarker chosen from the patient's eye-blink conditioning (EBC) response, the patient's mismatch negativity (MMN) amplitude, and the patient's auditory steady state response (ASSR) gamma band power.
163. The use according to any one of Embodiments 154 to 162, wherein the patient suffers from a disease chosen from attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
164. The use according to any one of Embodiments 154 to 162, wherein the patient suffers from a disease chosen from psychosis, schizophreniform disorder, schizoaffective disorder, and schizophrenia unassociated with aggression.
165. The use according to any one of Embodiments 154 to 162, wherein the patient has no more than moderate-severe positive symptoms associated with schizophrenia (<5 rating on PANSS positive symptom items P1, P3, P4, P5, P6).

### Non-Limiting Example Embodiments 2

Without limitation, some embodiments/clauses of the disclosure include:
1. A method of treating cognitive impairment associated with schizophrenia in a patient in need thereof comprising administering to the patient 50 mg to 500 mg of at least one compound chosen from Compound (I):

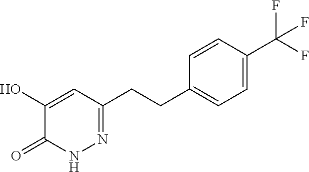 [SMILES: O=c1[nH]nc(CCc2ccc(C(F)(F)F)cc2)cc1O]

and pharmaceutically acceptable salts thereof once daily, wherein at least one cognitive symptom associated with schizophrenia in the patient is treated by the administration.
2. The method of treating cognitive impairment associated with schizophrenia according to Clause 1, wherein the method comprises:
   administering to the patient 50 mg to 500 mg of at least one compound chosen from Compound (I) and pharmaceutically acceptable salts thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient 50 mg to 500 mg of at least one compound chosen from Compound (I) and pharmaceutically acceptable salts thereof once daily if the 6 to 8 day administration improved at least one biomarker.
3. The method of treating cognitive impairment associated with schizophrenia according to Clause 1 or 2, wherein the at least one cognitive symptom associated with schizophrenia is chosen from impaired verbal memory, impaired working memory, impaired motor function, impaired attention, impaired processing speed, impaired verbal fluency, and impaired executive function.
4. The method of treating cognitive impairment associated with schizophrenia according to any one of Clauses 1 to 3, wherein the administration improves the patient's Brief Assessment of Cognition in Schizophrenia (BACS) Composite Score relative to a BACS Composite Score measured for the patient prior to the administration.
5. The method of treating cognitive impairment associated with schizophrenia according to any one of Clauses 1 to 4, wherein the at least one cognitive symptom associated with schizophrenia is chosen from impaired attention, impaired memory, impaired reasoning, impaired problem solving, impaired working memory, impaired processing speed, impaired language functions, and impaired social cognition.
6. The method of treating cognitive impairment associated with schizophrenia according to any one of Clauses 1 to 5, wherein the administration improves the patient's Schizophrenia Cognition Rating Scale (SCORS) interviewer total score relative to a SCORS interviewer total score measured for the patient prior to the administration.
7. The method of treating cognitive impairment associated with schizophrenia according to any one of Clauses 1 to 6, wherein the patient has at least one stable negative symptom associated with schizophrenia prior to the administration.
8. The method of treating cognitive impairment associated with schizophrenia according to Clause 7, wherein the at least one stable negative symptom associated with schizophrenia is chosen from anhedonia, loss of motivation, and reduced interest in social interaction.
9. The method of treating cognitive impairment associated with schizophrenia according to Clause 7, wherein the stability of the at least one stable negative symptom associated with schizophrenia is assessed using PANSS.
10. The method of treating cognitive impairment associated with schizophrenia according to Clause 7 or 8, wherein the stability of the at least one stable negative symptom associated with schizophrenia is assessed using the Brief Negative Symptom Scale (BNSS) instrument.
11. The method of treating cognitive impairment associated with schizophrenia according to any one of Clauses 7 to 10, wherein the at least one stable negative symptom associated with schizophrenia is stable for at least one month prior to the administration.
12. The method of treating cognitive impairment associated with schizophrenia according to any one of Clauses 1 to 6, wherein there is not statistically significant improvement in any negative symptom associated with schizophrenia in the patient following the administration.
13. The method of treating cognitive impairment associated with schizophrenia according to any one of Clauses 1 to 12, wherein the at least one compound is administered in combination with at least one additional active agent.
14. The method of treating cognitive impairment associated with schizophrenia according to Clause 13, wherein the at least one additional active agent treats at least one negative symptom associated with schizophrenia in the patient.
15. The method of treating cognitive impairment associated with schizophrenia according to Clause 14, wherein the at least one negative symptom associated with schizophrenia is chosen from anhedonia, loss of motivation, and reduced interest in social interaction.
16. The method of treating cognitive impairment associated with schizophrenia according to Clause 13, wherein the at least one additional therapeutic agent is chosen from aripiprazole, olanzapine, risperidone, and quetiapine fumarate.
17. The method of treating cognitive impairment associated with schizophrenia according to any one of Clauses 1 to 16, wherein the at least one compound is administered to the patient for more than 14 weeks.
18. The method of treating cognitive impairment associated with schizophrenia according to any one of Clauses 1 to 17, wherein the at least one compound is administered to the patient for more than 20 weeks.
19. The method of treating cognitive impairment associated with schizophrenia according to any one of Clauses 1 to 18, wherein the patient is administered 50 mg of the at least one compound once daily.
20. The method of treating cognitive impairment associated with schizophrenia according to any one of Clauses 1 to 19, wherein the patient is administered 125 mg of the at least one compound once daily.
21. The method of treating cognitive impairment associated with schizophrenia according to any one of Clauses 1 to 20, wherein the administration does not treat at least one negative symptom associated with schizophrenia.
22. The method of treating cognitive impairment associated with schizophrenia according to Clause 21, wherein the at least one negative symptom associated with schizophrenia is chosen from anhedonia, loss of motivation, and reduced interest in social interaction.
23. A method of treating cognitive impairment in a patient in need thereof comprising administering to the patient a therapeutically effective amount of at least one compound chosen from Compound (I):

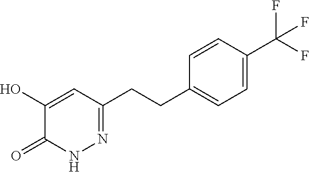 [SMILES: O=c1[nH]nc(CCc2ccc(C(F)(F)F)cc2)cc1O]

and pharmaceutically acceptable salts thereof, wherein the patient exhibits at least one cognitive symptom prior to the administration.
24. The method of treating cognitive impairment according to Clause 23, wherein the method comprises:
   administering to the patient a therapeutically effective amount of at least one compound chosen from Compound (I) and pharmaceutically acceptable salts thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient a therapeutically effective amount of at least one compound chosen from Compound (I) and pharmaceutically acceptable salts thereof once daily if the 6 to 8 day administration improved at least one biomarker.
25. The method of treating cognitive impairment according to Clause 23 or 24, wherein the at least one cognitive symptom is chosen from impaired verbal memory, impaired working memory, impaired motor function, impaired attention, impaired processing speed, impaired verbal fluency, and impaired executive function.
26. The method of treating cognitive impairment according to any one of Clauses 23 to 25, wherein the at least one cognitive symptom is chosen from impaired attention, impaired memory, impaired reasoning, impaired problem solving, impaired working memory, impaired processing speed, impaired language functions, and impaired social cognition.
27. The method of treating cognitive impairment according to any one of Clauses 23 to 26, wherein the administration treats the at least one cognitive symptom.
28. The method of treating cognitive impairment according to any one of Clauses 23 to 27, wherein the at least one cognitive symptom is associated with a psychotic disorder.
29. The method of treating cognitive impairment according to Clause 28, wherein the psychotic disorder is chosen from psychosis, schizophreniform disorder, schizoaffective disorder, and schizophrenia unassociated with aggression.
30. The method of treating cognitive impairment according to Clause 28 or 29, wherein the psychotic disorder is schizophreniform disorder, schizoaffective disorder, and schizophrenia unassociated with aggression.
31. The method of treating cognitive impairment according to any one of Clauses 28 to 30, wherein the psychotic disorder is schizophrenia unassociated with aggression.
32. The method of treating cognitive impairment according to any one of Clauses 23 to 28, wherein the at least one cognitive symptom is associated with attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
33. The method of treating cognitive impairment according to any one of Clauses 23 to 28, wherein the at least one cognitive symptom is associated with Cushing's disease, Lewy body disease, multiple sclerosis, stroke, addictive disorder, pervasive development disorder, fragile X syndrome, Prader-Willi syndrome, and delirium.
34. The method of treating cognitive impairment according to any one of Clauses 23 to 33, wherein the patient is administered 50 mg of the at least one compound once daily.
35. The method of treating cognitive impairment according to any one of Clauses 23 to 34, wherein the at least one compound is administered in combination with at least one additional active agent.
36. The method of treating cognitive impairment according to Clause 35, wherein the patient has schizophrenia and the at least one additional active agent treats at least one negative symptom in the patient.
37. The method of treating cognitive impairment according to Clause 36, wherein the at least one negative symptom is chosen from anhedonia, loss of motivation, and reduced interest in social interaction.
38. The method of treating cognitive impairment according to any one of Clauses 23 to 37, wherein the patient has at least one stable negative symptom chosen from anhedonia, loss of motivation, and reduced interest in social interaction prior to the administration.
39. The method of treating cognitive impairment according to any one of Clauses 1 to 38, wherein the at least one compound is administered in the form of at least one film-coated tablet.
40. A method of treating at least one cognitive symptom associated with schizophrenia in a patient, wherein the patient has no more than moderate-severe positive symptoms associated with schizophrenia (<5 rating on PANSS positive symptom items P1, P3, P4, P5, P6), comprising administering to the patient 50 mg to 500 mg of at least one compound chosen from Compound (I):

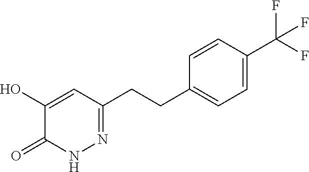 [SMILES: O=c1[nH]nc(CCc2ccc(C(F)(F)F)cc2)cc1O]

and pharmaceutically acceptable salts thereof once daily, wherein at least one cognitive symptom associated with schizophrenia in the patient is treated by the administration.
41. The method of treating at least one cognitive symptom associated with schizophrenia according to Clause 40, wherein the method comprises:
   administering to the patient 50 mg to 500 mg of at least one compound chosen from Compound (I) and pharmaceutically acceptable salts thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient 50 mg to 500 mg of at least one compound chosen from Compound (I) and pharmaceutically acceptable salts thereof once daily if the 6 to 8 day administration improved at least one biomarker.
42. The method of treating at least one cognitive symptom associated with schizophrenia according to Clause 40 or 41, wherein the administration improves the patient's Brief Assessment of Cognition in Schizophrenia (BACS) Composite Score relative to a BACS Composite Score measured for the patient prior to the administration.
43. The method of treating at least one cognitive symptom associated with schizophrenia according to any one of Clauses 40 to 42, wherein the administration improves the patient's Schizophrenia Cognition Rating Scale (SCORS) interviewer total score relative to a SCORS interviewer total score measured for the patient prior to the administration.
44. The method of treating at least one cognitive symptom associated with schizophrenia according to any one of Clauses 40 to 43, wherein there is not statistically significant improvement in any negative symptom associated with schizophrenia in the patient following the administration.
45. The method of treating at least one cognitive symptom associated with schizophrenia according to any one of Clauses 40 to 44, wherein the administration does not treat at least one negative symptom associated with schizophrenia.
46. The method of treating at least one cognitive symptom associated with schizophrenia according to Clause 45, wherein the at least one negative symptom associated with schizophrenia is chosen from anhedonia, loss of motivation, and reduced interest in social interaction.
47. The method of treating at least one cognitive symptom associated with schizophrenia according to any one of Clauses 40 to 46, wherein the patient is administered 50 mg of the at least one compound once daily.
48. A method of increasing D-serine levels, long-term potentiation, and/or synaptic plasticity in a patient in need thereof comprising administering to the patient less than 500 mg of at least one compound chosen from Compound (I):

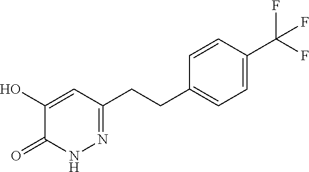 [SMILES: O=c1[nH]nc(CCc2ccc(C(F)(F)F)cc2)cc1O]

and pharmaceutically acceptable salts thereof once daily.
49. The method according to Clause 48, wherein the patient is administered 50 mg of the at least one compound once daily.
50. The method according to Clause 48 or 49, wherein the method increases D-serine levels in the patient.
51. The method according to any one of Clauses 48 to 50, wherein the method increases long-term potentiation in the patient.
52. The method according to any one of Clauses 48 to 51, wherein the method increases synaptic plasticity in the patient.
53. The method according to any one of Clauses 48 to 52, wherein the patient exhibits at least one cognitive symptom prior to the administration.
54. The method according to Clause 53, wherein the at least one cognitive symptom is chosen from impaired verbal memory, impaired working memory, impaired motor function, impaired attention, impaired processing speed, impaired verbal fluency, and impaired executive function.
55. The method according to Clause 53, wherein the at least one cognitive symptom is chosen from impaired attention, impaired memory, impaired reasoning, impaired problem solving, impaired working memory, impaired processing speed, impaired language functions, and impaired social cognition.
56. The method according to any one of Clauses 48 to 55, wherein the administration improves at least one biomarker chosen from the patient's eye-blink conditioning (EBC) response, the patient's mismatch negativity (MMN) amplitude, and the patient's auditory steady state response (ASSR) gamma band power.
57. The method according to any one of Clauses 48 to 56, wherein the patient suffers from a disease chosen from attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
58. The method according to any one of Clauses 48 to 56, wherein the patient suffers from a disease chosen from psychosis, schizophreniform disorder, schizoaffective disorder, and schizophrenia unassociated with aggression.
59. The method according to any one of Clauses 48 to 56, wherein the patient has no more than moderate-severe positive symptoms associated with schizophrenia (<5 rating on PANSS positive symptom items P1, P3, P4, P5, P6).

### Non-Limiting Example Embodiments 3

Without limitation, some embodiments/features of the disclosure include:
1. A method of treating cognitive impairment associated with schizophrenia in a patient in need thereof comprising administering to the patient 1 mg to 500 mg of at least one compound chosen from Compound (I):

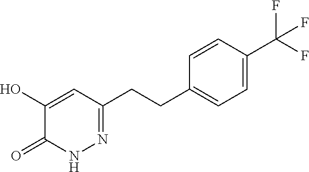 [SMILES: O=c1[nH]nc(CCc2ccc(C(F)(F)F)cc2)cc1O]

and pharmaceutically acceptable salts thereof once daily, wherein at least one cognitive symptom associated with schizophrenia in the patient is treated by the administration.
2. The method of treating cognitive impairment associated with schizophrenia according to Feature 1, wherein the method comprises:
   administering to the patient 1 mg to 500 mg of at least one compound chosen from Compound (I) and pharmaceutically acceptable salts thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient 1 mg to 500 mg of at least one compound chosen from Compound (I) and pharmaceutically acceptable salts thereof once daily if the 6 to 8 day administration improved at least one biomarker.
3. The method of treating cognitive impairment associated with schizophrenia according to Feature 1 or 2, wherein the at least one cognitive symptom associated with schizophrenia is chosen from impaired verbal memory, impaired working memory, impaired motor function, impaired attention, impaired processing speed, impaired verbal fluency, and impaired executive function.
4. The method of treating cognitive impairment associated with schizophrenia according to any one of Features 1 to 3, wherein the administration improves the patient's Brief Assessment of Cognition in Schizophrenia (BACS) Composite Score relative to a BACS Composite Score measured for the patient prior to the administration.
5. The method of treating cognitive impairment associated with schizophrenia according to any one of Features 1 to 4, wherein the at least one cognitive symptom associated with schizophrenia is chosen from impaired attention, impaired memory, impaired reasoning, impaired problem solving, impaired working memory, impaired processing speed, impaired language functions, and impaired social cognition.
6. The method of treating cognitive impairment associated with schizophrenia according to any one of Features 1 to 5, wherein the administration improves the patient's Schizophrenia Cognition Rating Scale (SCORS) interviewer total score relative to a SCORS interviewer total score measured for the patient prior to the administration.
7. The method of treating cognitive impairment associated with schizophrenia according to any one of Features 1 to 6, wherein the administration improves at least one metric chosen from:
   the patient's performance on the Continuous Performance Test-Identical Pairs (CPT-IP) test relative to a CPT-IP test prior to the administration;
   the patient's performance on the Brief Visuospatial Memory Test-Revised (BVMT-R) test relative to the patient's performance on a BVMT-R test prior to the administration;
   the patient's performance on the Mayer-Salovey-Caruso Emotional Intelligence Test (MSCEIT) relative to the patient's performance on a MSCEIT prior to the administration;
   the patient's performance on the Virtual Reality Functional Capacity Assessment Tool (VRFCAT) relative to the patient's performance on a VRFCAT prior to the administration; and
   the patient's Clinical Global Impression-Severity Scale (CGI-S) score relative to a CGI-S score measured for the patient prior to the administration.
8. The method of treating cognitive impairment associated with schizophrenia according to any one of Features 1 to 7, wherein the patient has at least one stable negative symptom associated with schizophrenia prior to the administration.
9. The method of treating cognitive impairment associated with schizophrenia according to Feature 8, wherein the at least one stable negative symptom associated with schizophrenia is chosen from anhedonia, loss of motivation, and reduced interest in social interaction.
10. The method of treating cognitive impairment associated with schizophrenia according to Feature 8, wherein the stability of the at least one stable negative symptom associated with schizophrenia is assessed using PANSS.
11. The method of treating cognitive impairment associated with schizophrenia according to Feature 8 or 9, wherein the stability of the at least one stable negative symptom associated with schizophrenia is assessed using the Brief Negative Symptom Scale (BNSS) instrument.
12. The method of treating cognitive impairment associated with schizophrenia according to any one of Features 8 to 11, wherein the at least one stable negative symptom associated with schizophrenia is stable for at least one month prior to the administration.
13. The method of treating cognitive impairment associated with schizophrenia according to any one of Features 1 to 7, wherein there is not statistically significant improvement in any negative symptom associated with schizophrenia in the patient following the administration.
14. The method of treating cognitive impairment associated with schizophrenia according to any one of Features 1 to 13, wherein the at least one compound is administered in combination with at least one additional active agent.
15. The method of treating cognitive impairment associated with schizophrenia according to Feature 14, wherein the at least one additional active agent treats at least one negative symptom associated with schizophrenia in the patient.
16. The method of treating cognitive impairment associated with schizophrenia according to Feature 15, wherein the at least one negative symptom associated with schizophrenia is chosen from anhedonia, loss of motivation, and reduced interest in social interaction.
17. The method of treating cognitive impairment associated with schizophrenia according to Feature 14, wherein the at least one additional therapeutic agent is chosen from aripiprazole, olanzapine, risperidone, and quetiapine fumarate.
18. The method of treating cognitive impairment associated with schizophrenia according to any one of Features 1 to 17, wherein the at least one compound is administered to the patient for more than 14 weeks.
19. The method of treating cognitive impairment associated with schizophrenia according to any one of Features 1 to 18, wherein the at least one compound is administered to the patient for more than 20 weeks.
20. The method of treating cognitive impairment associated with schizophrenia according to any one of Features 1 to 19, wherein the patient is administered 1 mg to 100 mg of the at least one compound once daily.
21. The method of treating cognitive impairment associated with schizophrenia according to any one of Features 1 to 19, wherein the patient is administered 20 mg or 50 mg of the at least one compound once daily.
22. The method of treating cognitive impairment associated with schizophrenia according to any one of Features 1 to 19, wherein the patient is administered 20 mg of the at least one compound once daily.
23. The method of treating cognitive impairment associated with schizophrenia according to any one of Features 1 to 19, wherein the patient is administered 50 mg of the at least one compound once daily.
24. The method of treating cognitive impairment associated with schizophrenia according to any one of Features 1 to 23, wherein the administration does not treat at least one negative symptom associated with schizophrenia.
25. The method of treating cognitive impairment associated with schizophrenia according to Feature 24, wherein the at least one negative symptom associated with schizophrenia is chosen from anhedonia, loss of motivation, and reduced interest in social interaction.
26. The method of treating cognitive impairment associated with schizophrenia according to any one of Features 1 to 25, wherein the patient is on a stable regimen of psychotropic medications.
27. The method of treating cognitive impairment associated with schizophrenia according to any one of Features 1 to 26, wherein the patient was diagnosed with schizophrenia at least one year prior to the administration.
28. The method of treating cognitive impairment associated with schizophrenia according to any one of Features 1 to 27, wherein the patient has stable symptomatology for at least 3 months prior to the administration.
29. A method of treating cognitive impairment in a patient in need thereof comprising administering to the patient a therapeutically effective amount of at least one compound chosen from Compound (I):

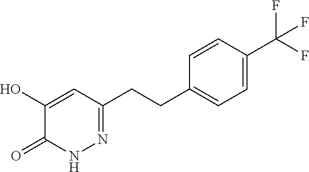 [SMILES: O=c1[nH]nc(CCc2ccc(C(F)(F)F)cc2)cc1O]

and pharmaceutically acceptable salts thereof, wherein the patient exhibits at least one cognitive symptom prior to the administration.
30. The method of treating cognitive impairment according to Feature 29, wherein the method comprises:
   administering to the patient a therapeutically effective amount of at least one compound chosen from Compound (I) and pharmaceutically acceptable salts thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient a therapeutically effective amount of at least one compound chosen from Compound (I) and pharmaceutically acceptable salts thereof once daily if the 6 to 8 day administration improved at least one biomarker.
31. The method of treating cognitive impairment according to Feature 29 or 30, wherein the at least one cognitive symptom is chosen from impaired verbal memory, impaired working memory, impaired motor function, impaired attention, impaired processing speed, impaired verbal fluency, and impaired executive function.
32. The method of treating cognitive impairment according to any one of Features 29 to 31, wherein the at least one cognitive symptom is chosen from impaired attention, impaired memory, impaired reasoning, impaired problem solving, impaired working memory, impaired processing speed, impaired language functions, and impaired social cognition.
33. The method of treating cognitive impairment according to any one of Features 29 to 32, wherein the administration treats the at least one cognitive symptom.
34. The method of treating cognitive impairment according to any one of Features 29 to 33, wherein the at least one cognitive symptom is associated with a psychotic disorder.
35. The method of treating cognitive impairment according to Feature 34, wherein the psychotic disorder is chosen from psychosis, schizophreniform disorder, schizoaffective disorder, and schizophrenia unassociated with aggression.
36. The method of treating cognitive impairment according to Feature 34 or 35, wherein the psychotic disorder is schizophreniform disorder, schizoaffective disorder, and schizophrenia unassociated with aggression.
37. The method of treating cognitive impairment according to any one of Features 34 to 36, wherein the psychotic disorder is schizophrenia unassociated with aggression.
38. The method of treating cognitive impairment according to any one of Features 29 to 34, wherein the at least one cognitive symptom is associated with attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
39. The method of treating cognitive impairment according to any one of Features 29 to 34, wherein the at least one cognitive symptom is associated with Cushing's disease, Lewy body disease, multiple sclerosis, stroke, addictive disorder, pervasive development disorder, fragile X syndrome, Prader-Willi syndrome, and delirium.
40. The method of treating cognitive impairment according to any one of Features 29 to 39, wherein the patient is administered 50 mg of the at least one compound once daily.
41. The method of treating cognitive impairment according to any one of Features 29 to 39, wherein the patient is administered 20 mg of the at least one compound once daily.
42. The method of treating cognitive impairment according to any one of Features 29 to 41, wherein the at least one compound is administered in combination with at least one additional active agent.
43. The method of treating cognitive impairment according to Feature 42, wherein the patient has schizophrenia and the at least one additional active agent treats at least one negative symptom in the patient.
44. The method of treating cognitive impairment according to Feature 43, wherein the at least one negative symptom is chosen from anhedonia, loss of motivation, and reduced interest in social interaction.
45. The method of treating cognitive impairment according to any one of Features 29 to 44, wherein the patient has at least one stable negative symptom chosen from anhedonia, loss of motivation, and reduced interest in social interaction prior to the administration.
46. The method of treating cognitive impairment according to any one of Features 1 to 45, wherein the at least one compound is administered in the form of at least one film-coated tablet.
47. The method of treating cognitive impairment according to any one of Features 1 to 46, wherein the at least one compound is administered with water or milk.
48. A method of treating at least one cognitive symptom associated with schizophrenia in a patient, wherein the patient has no more than moderate-severe positive symptoms associated with schizophrenia (<5 rating on PANSS positive symptom items P1, P3, P4, P5, P6), comprising administering to the patient 1 mg to 500 mg of at least one compound chosen from Compound (I):

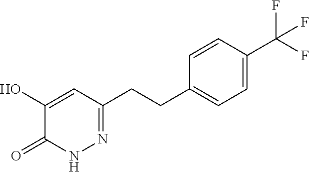 [SMILES: O=c1[nH]nc(CCc2ccc(C(F)(F)F)cc2)cc1O]

and pharmaceutically acceptable salts thereof once daily, wherein at least one cognitive symptom associated with schizophrenia in the patient is treated by the administration.
49. The method of treating at least one cognitive symptom associated with schizophrenia according to Feature 48, wherein the method comprises:
   administering to the patient 1 mg to 500 mg of at least one compound chosen from Compound (I) and pharmaceutically acceptable salts thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient 1 mg to 500 mg of at least one compound chosen from Compound (I) and pharmaceutically acceptable salts thereof once daily if the 6 to 8 day administration improved at least one biomarker.
50. The method of treating at least one cognitive symptom associated with schizophrenia according to Feature 48 or 49, wherein the administration improves the patient's Brief Assessment of Cognition in Schizophrenia (BACS) Composite Score relative to a BACS Composite Score measured for the patient prior to the administration.
51. The method of treating at least one cognitive symptom associated with schizophrenia according to any one of Features 48 to 50, wherein the administration improves the patient's Schizophrenia Cognition Rating Scale (SCORS) interviewer total score relative to a SCORS interviewer total score measured for the patient prior to the administration.
52. The method of treating at least one cognitive symptom associated with schizophrenia according to any one of Features 48 to 51, wherein the administration improves at least one metric chosen from:
   the patient's performance on the Continuous Performance Test-Identical Pairs (CPT-IP) test relative to a CPT-IP test prior to the administration;
   the patient's performance on the Brief Visuospatial Memory Test-Revised (BVMT-R) test relative to the patient's performance on a BVMT-R test prior to the administration;
   the patient's performance on the Mayer-Salovey-Caruso Emotional Intelligence Test (MSCEIT) relative to the patient's performance on a MSCEIT prior to the administration;
   the patient's performance on the Virtual Reality Functional Capacity Assessment Tool (VRFCAT) relative to the patient's performance on a VRFCAT prior to the administration; and
   the patient's Clinical Global Impression-Severity Scale (CGI-S) score relative to a CGI-S score measured for the patient prior to the administration.
53. The method of treating at least one cognitive symptom associated with schizophrenia according to any one of Features 48 to 52, wherein there is not statistically significant improvement in any negative symptom associated with schizophrenia in the patient following the administration.
54. The method of treating at least one cognitive symptom associated with schizophrenia according to any one of Features 48 to 53, wherein the administration does not treat at least one negative symptom associated with schizophrenia.
55. The method of treating at least one cognitive symptom associated with schizophrenia according to Feature 54, wherein the at least one negative symptom associated with schizophrenia is chosen from anhedonia, loss of motivation, and reduced interest in social interaction.
56. The method of treating at least one cognitive symptom associated with schizophrenia according to any one of Features 48 to 55, wherein the patient is administered 50 mg of the at least one compound once daily.
57. The method of treating at least one cognitive symptom associated with schizophrenia according to any one of Features 48 to 55, wherein the patient is administered 20 mg of the at least one compound once daily.
58. A method of increasing D-serine levels, long-term potentiation, and/or synaptic plasticity in a patient in need thereof comprising administering to the patient less than 500 mg of at least one compound chosen from Compound (I):

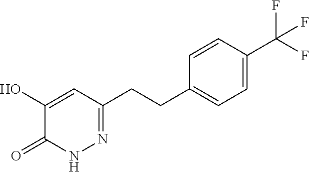 [SMILES: O=c1[nH]nc(CCc2ccc(C(F)(F)F)cc2)cc1O]

and pharmaceutically acceptable salts thereof once daily.
59. The method according to Feature 58, wherein the patient is administered 50 mg of the at least one compound once daily.
60. The method according to Feature 58, wherein the patient is administered 20 mg of the at least one compound once daily.
61. The method according to any one of Features 58 to 60, wherein the method increases D-serine levels in the patient.
62. The method according to any one of Features 58 to 61, wherein the method increases long-term potentiation in the patient.
63. The method according to any one of Features 58 to 62, wherein the method increases synaptic plasticity in the patient.
64. The method according to any one of Features 58 to 63, wherein the patient exhibits at least one cognitive symptom prior to the administration.
65. The method according to Feature 64, wherein the at least one cognitive symptom is chosen from impaired verbal memory, impaired working memory, impaired motor function, impaired attention, impaired processing speed, impaired verbal fluency, and impaired executive function.
66. The method according to Feature 64, wherein the at least one cognitive symptom is chosen from impaired attention, impaired memory, impaired reasoning, impaired problem solving, impaired working memory, impaired processing speed, impaired language functions, and impaired social cognition.
67. The method according to any one of Features 58 to 66, wherein the administration improves at least one biomarker chosen from the patient's eye-blink conditioning (EBC) response, the patient's mismatch negativity (MMN) amplitude, and the patient's auditory steady state response (ASSR) gamma band power.
68. The method according to any one of Features 58 to 67, wherein the patient suffers from a disease chosen from attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
69. The method according to any one of Features 58 to 67, wherein the patient suffers from a disease chosen from psychosis, schizophreniform disorder, schizoaffective disorder, and schizophrenia unassociated with aggression.
70. The method according to any one of Features 58 to 67, wherein the patient has no more than moderate-severe positive symptoms associated with schizophrenia (<5 rating on PANSS positive symptom items P1, P3, P4, P5, P6).
71.

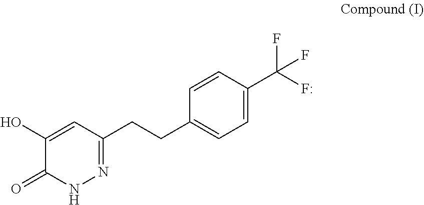

or a pharmaceutically acceptable salt thereof,
   for use in a method of treating cognitive impairment associated with schizophrenia in a patient in need thereof,
   the method comprising administering to the patient 1 mg to 500 mg of the Compound (I) or a pharmaceutically acceptable salt thereof once daily,
   wherein at least one cognitive symptom associated with schizophrenia in the patient is treated by the administration.
72. The Compound (I) or a pharmaceutically acceptable salt thereof for use according to Feature 71, wherein the method comprises:
   administering to the patient 1 mg to 500 mg of the Compound (I) or a pharmaceutically acceptable salt thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient 1 mg to 500 mg of the Compound (I) or a pharmaceutically acceptable salt thereof once daily if the 6 to 8 day administration improved at least one biomarker.
73. The Compound (I) or a pharmaceutically acceptable salt thereof for use according to Feature 71 or 72, wherein the at least one cognitive symptom associated with schizophrenia is chosen from impaired verbal memory, impaired working memory, impaired motor function, impaired attention, impaired processing speed, impaired verbal fluency, and impaired executive function.
74. The Compound (I) or a pharmaceutically acceptable salt thereof for use according to any one of Features 71 to 73, wherein the administration improves the patient's Brief Assessment of Cognition in Schizophrenia (BACS) Composite Score relative to a BACS Composite Score measured for the patient prior to the administration.
75. The Compound (I) or a pharmaceutically acceptable salt thereof for use according to any one of Features 71 to 74, wherein the at least one cognitive symptom associated with schizophrenia is chosen from impaired attention, impaired memory, impaired reasoning, impaired problem solving, impaired working memory, impaired processing speed, impaired language functions, and impaired social cognition.
76. The Compound (I) or a pharmaceutically acceptable salt thereof for use according to any one of Features 71 to 75, wherein the administration improves the patient's Schizophrenia Cognition Rating Scale (SCORS) interviewer total score relative to a SCORS interviewer total score measured for the patient prior to the administration.
77. The Compound (I) or a pharmaceutically acceptable salt thereof for use according to any one of Features 71 to 76, wherein the administration improves at least one metric chosen from:
   the patient's performance on the Continuous Performance Test-Identical Pairs (CPT-IP) test relative to a CPT-IP test prior to the administration;
   the patient's performance on the Brief Visuospatial Memory Test-Revised (BVMT-R) test relative to the patient's performance on a BVMT-R test prior to the administration;
   the patient's performance on the Mayer-Salovey-Caruso Emotional Intelligence Test (MSCEIT) relative to the patient's performance on a MSCEIT prior to the administration;
   the patient's performance on the Virtual Reality Functional Capacity Assessment Tool (VRFCAT) relative to the patient's performance on a VRFCAT prior to the administration; and
   the patient's Clinical Global Impression-Severity Scale (CGI-S) score relative to a CGI-S score measured for the patient prior to the administration.
78. The Compound (I) or a pharmaceutically acceptable salt thereof for use according to any one of Features 71 to 77, wherein the patient has at least one stable negative symptom associated with schizophrenia prior to the administration.
79. The Compound (I) or a pharmaceutically acceptable salt thereof for use according to Feature 78, wherein the at least one stable negative symptom associated with schizophrenia is chosen from anhedonia, loss of motivation, and reduced interest in social interaction.
80. The Compound (I) or a pharmaceutically acceptable salt thereof for use according to Feature 78, wherein the stability of the at least one stable negative symptom associated with schizophrenia is assessed using PANSS.
81. The Compound (I) or a pharmaceutically acceptable salt thereof for use according to Feature 78 or 79, wherein the stability of the at least one stable negative symptom associated with schizophrenia is assessed using the Brief Negative Symptom Scale (BNSS) instrument.
82. The Compound (I) or a pharmaceutically acceptable salt thereof for use according to any one of Features 78 to 81, wherein the at least one stable negative symptom associated with schizophrenia is stable for at least one month prior to the administration.
83. The Compound (I) or a pharmaceutically acceptable salt thereof for use according to any one of Features 71 to 77, wherein there is not statistically significant improvement in any negative symptom associated with schizophrenia in the patient following the administration.
84. The Compound (I) or a pharmaceutically acceptable salt thereof for use according to any one of Features 71 to 83, wherein the Compound (I) or a pharmaceutically acceptable salt thereof is administered in combination with at least one additional active agent.
85. The Compound (I) or a pharmaceutically acceptable salt thereof for use according to Feature 84, wherein the at least one additional active agent treats at least one negative symptom associated with schizophrenia in the patient.
86. The Compound (I) or a pharmaceutically acceptable salt thereof for use according to Feature 85, wherein the at least one negative symptom associated with schizophrenia is chosen from anhedonia, loss of motivation, and reduced interest in social interaction.
87. The Compound (I) or a pharmaceutically acceptable salt thereof for use according to Feature 84, wherein the at least one additional therapeutic agent is chosen from aripiprazole, olanzapine, risperidone, and quetiapine fumarate.
88. The Compound (I) or a pharmaceutically acceptable salt thereof for use according to any one of Features 71 to 87, wherein the Compound (I) or a pharmaceutically acceptable salt thereof is administered to the patient for more than 14 weeks.
89. The Compound (I) or a pharmaceutically acceptable salt thereof for use according to any one of Features 71 to 88, wherein the Compound (I) or a pharmaceutically acceptable salt thereof is administered to the patient for more than 20 weeks.
90. The Compound (I) or a pharmaceutically acceptable salt thereof for use according to any one of Features 71 to 89, wherein the patient is administered 1 mg to 100 mg of the Compound (I) or a pharmaceutically acceptable salt thereof once daily.
91. The Compound (I) or a pharmaceutically acceptable salt thereof for use according to any one of Features 71 to 89, wherein the patient is administered 20 mg or 50 mg of the Compound (I) or a pharmaceutically acceptable salt thereof once daily.
92. The Compound (I) or a pharmaceutically acceptable salt thereof for use according to any one of Features 71 to 89, wherein the patient is administered 20 mg of the Compound (I) or a pharmaceutically acceptable salt thereof once daily.
93. The Compound (I) or a pharmaceutically acceptable salt thereof for use according to any one of Features 71 to 89, wherein the patient is administered 50 mg of the Compound (I) or a pharmaceutically acceptable salt thereof once daily.
94. The Compound (I) or a pharmaceutically acceptable salt thereof for use according to any one of Features 71 to 93, wherein the administration does not treat at least one negative symptom associated with schizophrenia.
95. The Compound (I) or a pharmaceutically acceptable salt thereof for use according to Feature 94, wherein the at least one negative symptom associated with schizophrenia is chosen from anhedonia, loss of motivation, and reduced interest in social interaction.
96. The Compound (I) or a pharmaceutically acceptable salt thereof for use according to any one of Features 71 to 95, wherein the patient is on a stable regimen of psychotropic medications.
97. The Compound (I) or a pharmaceutically acceptable salt thereof for use according to any one of Features 71 to 96, wherein the patient was diagnosed with schizophrenia at least one year prior to the administration.
98. The Compound (I) or a pharmaceutically acceptable salt thereof for use according to any one of Features 71 to 97, wherein the patient has stable symptomatology for at least 3 months prior to the administration.
99.

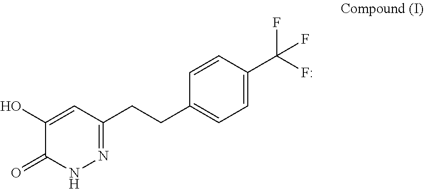

or a pharmaceutically acceptable salt thereof,
   for use in a method of treating cognitive impairment in a patient in need thereof,
   the method comprising administering to the patient a therapeutically effective amount of Compound (I) or a pharmaceutically acceptable salt thereof,
   wherein the patient exhibits at least one cognitive symptom prior to the administration.
100. The Compound (I) or a pharmaceutically acceptable salt thereof for use according to Feature 99, wherein the method comprises:
   administering to the patient a therapeutically effective amount of at least one compound chosen from the Compound (I) or a pharmaceutically acceptable salt thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient a therapeutically effective amount of the Compound (I) or a pharmaceutically acceptable salt thereof once daily if the 6 to 8 day administration improved at least one biomarker.
101. The Compound (I) or a pharmaceutically acceptable salt thereof for use according to Feature 99 or 100, wherein the at least one cognitive symptom is chosen from impaired verbal memory, impaired working memory, impaired motor function, impaired attention, impaired processing speed, impaired verbal fluency, and impaired executive function.
102. The Compound (I) or a pharmaceutically acceptable salt thereof for use according to any one of Features 99 to 101, wherein the at least one cognitive symptom is chosen from impaired attention, impaired memory, impaired reasoning, impaired problem solving, impaired working memory, impaired processing speed, impaired language functions, and impaired social cognition.
103. The Compound (I) or a pharmaceutically acceptable salt thereof for use according to any one of Features 99 to 102, wherein the administration treats the at least one cognitive symptom.
104. The Compound (I) or a pharmaceutically acceptable salt thereof for use according to any one of Features 99 to 103, wherein the at least one cognitive symptom is associated with a psychotic disorder.
105. The Compound (I) or a pharmaceutically acceptable salt thereof for use according to Feature 104, wherein the psychotic disorder is chosen from psychosis, schizophreniform disorder, schizoaffective disorder, and schizophrenia unassociated with aggression.
106. The Compound (I) or a pharmaceutically acceptable salt thereof for use according to Feature 104 or 105, wherein the psychotic disorder is schizophreniform disorder, schizoaffective disorder, and schizophrenia unassociated with aggression.
107. The Compound (I) or a pharmaceutically acceptable salt thereof for use according to any one of Features 104 to 106, wherein the psychotic disorder is schizophrenia unassociated with aggression.
108. The Compound (I) or a pharmaceutically acceptable salt thereof for use according to any one of Features 99 to 104, wherein the at least one cognitive symptom is associated with attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
109. The Compound (I) or a pharmaceutically acceptable salt thereof for use according to any one of Features 99 to 104, wherein the at least one cognitive symptom is associated with Cushing's disease, Lewy body disease, multiple sclerosis, stroke, addictive disorder, pervasive development disorder, fragile X syndrome, Prader-Willi syndrome, and delirium.
110. The Compound (I) or a pharmaceutically acceptable salt thereof for use according to any one of Features 99 to 109, wherein the patient is administered 50 mg of the Compound (I) or a pharmaceutically acceptable salt thereof once daily.
111. The Compound (I) or a pharmaceutically acceptable salt thereof for use according to any one of Features 99 to 109, wherein the patient is administered 20 mg of the Compound (I) or a pharmaceutically acceptable salt thereof once daily.
112. The Compound (I) or a pharmaceutically acceptable salt thereof for use according to any one of Features 99 to 111, wherein the Compound (I) or a pharmaceutically acceptable salt thereof is administered in combination with at least one additional active agent.
113. The Compound (I) or a pharmaceutically acceptable salt thereof for use according to Feature 112, wherein the patient has schizophrenia and the at least one additional active agent treats at least one negative symptom in the patient.
114. The Compound (I) or a pharmaceutically acceptable salt thereof for use according to Feature 113, wherein the at least one negative symptom is chosen from anhedonia, loss of motivation, and reduced interest in social interaction.
115. The Compound (I) or a pharmaceutically acceptable salt thereof for use according to any one of Features 99 to 114, wherein the patient has at least one stable negative symptom chosen from anhedonia, loss of motivation, and reduced interest in social interaction prior to the administration.
116. The Compound (I) or a pharmaceutically acceptable salt thereof for use according to any one of Features 71 to 115, wherein the Compound (I) or a pharmaceutically acceptable salt thereof is administered in the form of at least one film-coated tablet.
117. The Compound (I) or a pharmaceutically acceptable salt thereof for use according to any one of Features 71 to 116, wherein the Compound (I) or a pharmaceutically acceptable salt thereof is administered with water or milk.
118.

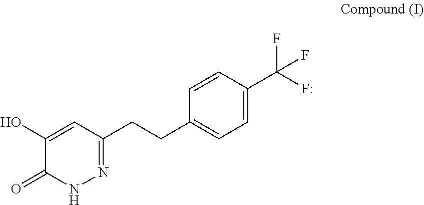

or a pharmaceutically acceptable salt thereof,
   for use in a method of treating at least one cognitive symptom associated with schizophrenia in a patient,
   wherein the patient has no more than moderate-severe positive symptoms associated with schizophrenia (<5 rating on PANSS positive symptom items P1, P3, P4, P5, P6),
   the method comprising administering to the patient 1 mg to 500 mg of the Compound (I) or a pharmaceutically acceptable salt thereof for use once daily.
119. The Compound (I) or a pharmaceutically acceptable salt thereof for use according to Feature 118, wherein the method comprises:
   administering to the patient 1 mg to 500 mg of the Compound (I) or a pharmaceutically acceptable salt thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient 1 mg to 500 mg of the Compound (I) or a pharmaceutically acceptable salt thereof once daily if the 6 to 8 day administration improved at least one biomarker.
120. The Compound (I) or a pharmaceutically acceptable salt thereof for use according to Feature 118 or 119, wherein the administration improves the patient's Brief Assessment of Cognition in Schizophrenia (BACS) Composite Score relative to a BACS Composite Score measured for the patient prior to the administration.
121. The Compound (I) or a pharmaceutically acceptable salt thereof for use according to any one of Features 118 to 120, wherein the administration improves the patient's Schizophrenia Cognition Rating Scale (SCORS) interviewer total score relative to a SCORS interviewer total score measured for the patient prior to the administration.
122. The Compound (I) or a pharmaceutically acceptable salt thereof for use according to any one of Features 118 to 121, wherein the administration improves at least one metric chosen from:
   the patient's performance on the Continuous Performance Test-Identical Pairs (CPT-IP) test relative to a CPT-IP test prior to the administration;
   the patient's performance on the Brief Visuospatial Memory Test-Revised (BVMT-R) test relative to the patient's performance on a BVMT-R test prior to the administration;
   the patient's performance on the Mayer-Salovey-Caruso Emotional Intelligence Test (MSCEIT) relative to the patient's performance on a MSCEIT prior to the administration;
   the patient's performance on the Virtual Reality Functional Capacity Assessment Tool (VRFCAT) relative to the patient's performance on a VRFCAT prior to the administration; and
   the patient's Clinical Global Impression-Severity Scale (CGI-S) score relative to a CGI-S score measured for the patient prior to the administration.
123. The Compound (I) or a pharmaceutically acceptable salt thereof for use according to any one of Features 118 to 122, wherein there is not statistically significant improvement in any negative symptom associated with schizophrenia in the patient following the administration.
124. The Compound (I) or a pharmaceutically acceptable salt thereof for use according to any one of Features 118 to 123, wherein the administration does not treat at least one negative symptom associated with schizophrenia.
125. The Compound (I) or a pharmaceutically acceptable salt thereof for use according to Feature 124, wherein the at least one negative symptom associated with schizophrenia is chosen from anhedonia, loss of motivation, and reduced interest in social interaction.
126. The Compound (I) or a pharmaceutically acceptable salt thereof for use according to any one of Features 118 to 125, wherein the patient is administered 50 mg of the Compound (I) or a pharmaceutically acceptable salt thereof once daily.
127. The Compound (I) or a pharmaceutically acceptable salt thereof for use according to any one of Features 118 to 125, wherein the patient is administered 20 mg of the Compound (I) or a pharmaceutically acceptable salt thereof once daily.
128.

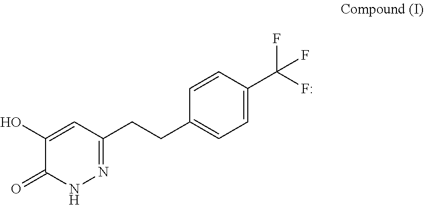

or a pharmaceutically acceptable salt thereof,
   for use in a method of increasing D-serine levels, long-term potentiation, and/or synaptic plasticity in a patient in need thereof,
   the method comprising administering to the patient less than 500 mg of the Compound (I) or a pharmaceutically acceptable salt thereof once daily.
129. The Compound (I) or a pharmaceutically acceptable salt thereof for use according to Feature 128, wherein the patient is administered 50 mg of the Compound (I) or a pharmaceutically acceptable salt thereof once daily.
130. The Compound (I) or a pharmaceutically acceptable salt thereof for use according to Feature 128, wherein the patient is administered 20 mg of the Compound (I) or a pharmaceutically acceptable salt thereof once daily.
131. The Compound (I) or a pharmaceutically acceptable salt thereof for use according to any one of Features 128 to 130, wherein the method increases D-serine levels in the patient.
132. The Compound (I) or a pharmaceutically acceptable salt thereof for use according to any one of Features 128 to 131, wherein the method increases long-term potentiation in the patient.
133. The Compound (I) or a pharmaceutically acceptable salt thereof for use according to any one of Features 128 to 132, wherein the method increases synaptic plasticity in the patient.
134. The Compound (I) or a pharmaceutically acceptable salt thereof for use according to any one of Features 128 to 133, wherein the patient exhibits at least one cognitive symptom prior to the administration.
135. The Compound (I) or a pharmaceutically acceptable salt thereof for use according to Feature 134, wherein the at least one cognitive symptom is chosen from impaired verbal memory, impaired working memory, impaired motor function, impaired attention, impaired processing speed, impaired verbal fluency, and impaired executive function.
136. The Compound (I) or a pharmaceutically acceptable salt thereof for use according to Feature 134, wherein the at least one cognitive symptom is chosen from impaired attention, impaired memory, impaired reasoning, impaired problem solving, impaired working memory, impaired processing speed, impaired language functions, and impaired social cognition.
137. The Compound (I) or a pharmaceutically acceptable salt thereof for use according to any one of Features 128 to 136, wherein the administration improves at least one biomarker chosen from the patient's eye-blink conditioning (EBC) response, the patient's mismatch negativity (MMN) amplitude, and the patient's auditory steady state response (ASSR) gamma band power.
138. The Compound (I) or a pharmaceutically acceptable salt thereof for use according to any one of Features 128 to 137, wherein the patient suffers from a disease chosen from attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
139. The Compound (I) or a pharmaceutically acceptable salt thereof for use according to any one of Features 128 to 137, wherein the patient suffers from a disease chosen from psychosis, schizophreniform disorder, schizoaffective disorder, and schizophrenia unassociated with aggression.
140. The Compound (I) or a pharmaceutically acceptable salt thereof for use according to any one of Features 128 to 137, wherein the patient has no more than moderate-severe positive symptoms associated with schizophrenia (<5 rating on PANSS positive symptom items P1, P3, P4, P5, P6).
141. Use of Compound (I):

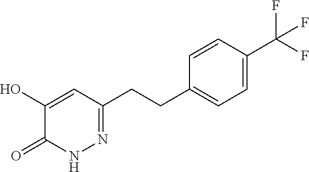 [SMILES: O=c1[nH]nc(CCc2ccc(C(F)(F)F)cc2)cc1O]

or a pharmaceutically acceptable salt thereof,
   in the manufacture of a medicament for use in a method of treating cognitive impairment associated with schizophrenia in a patient in need thereof,
   wherein the method comprises administering to the patient 1 mg to 500 mg of the Compound (I) or a pharmaceutically acceptable salt thereof once daily,
   wherein at least one cognitive symptom associated with schizophrenia in the patient is treated by the administration.
142. The use according to Feature 141, wherein the method comprises:
   administering to the patient 1 mg to 500 mg of the Compound (I) or a pharmaceutically acceptable salt thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient 1 mg to 500 mg of the Compound (I) or a pharmaceutically acceptable salt thereof once daily if the 6 to 8 day administration improved at least one biomarker.
143. The use according to Feature 141 or 142, wherein the at least one cognitive symptom associated with schizophrenia is chosen from impaired verbal memory, impaired working memory, impaired motor function, impaired attention, impaired processing speed, impaired verbal fluency, and impaired executive function.
144. The use according to any one of Features 141 to 143, wherein the administration improves the patient's Brief Assessment of Cognition in Schizophrenia (BACS) Composite Score relative to a BACS Composite Score measured for the patient prior to the administration.
145. The use according to any one of Features 141 to 144, wherein the at least one cognitive symptom associated with schizophrenia is chosen from impaired attention, impaired memory, impaired reasoning, impaired problem solving, impaired working memory, impaired processing speed, impaired language functions, and impaired social cognition.
146. The use according to any one of Features 141 to 145, wherein the administration improves the patient's Schizophrenia Cognition Rating Scale (SCORS) interviewer total score relative to a SCORS interviewer total score measured for the patient prior to the administration.
147. The use according to any one of Features 141 to 146, wherein the administration improves at least one metric chosen from:
   the patient's performance on the Continuous Performance Test-Identical Pairs (CPT-IP) test relative to a CPT-IP test prior to the administration;
   the patient's performance on the Brief Visuospatial Memory Test-Revised (BVMT-R) test relative to the patient's performance on a BVMT-R test prior to the administration;
   the patient's performance on the Mayer-Salovey-Caruso Emotional Intelligence Test (MSCEIT) relative to the patient's performance on a MSCEIT prior to the administration;
   the patient's performance on the Virtual Reality Functional Capacity Assessment Tool (VRFCAT) relative to the patient's performance on a VRFCAT prior to the administration; and
   the patient's Clinical Global Impression-Severity Scale (CGI-S) score relative to a CGI-S score measured for the patient prior to the administration.
148. The use according to any one of Features 141 to 147, wherein the patient has at least one stable negative symptom associated with schizophrenia prior to the administration.
149. The use according to Feature 148, wherein the at least one stable negative symptom associated with schizophrenia is chosen from anhedonia, loss of motivation, and reduced interest in social interaction.
150. The use according to Feature 148, wherein the stability of the at least one stable negative symptom associated with schizophrenia is assessed using PANSS.
151. The use according to Feature 148 or 149, wherein the stability of the at least one stable negative symptom associated with schizophrenia is assessed using the Brief Negative Symptom Scale (BNSS) instrument.
152. The use according to any one of Features 148 to 151, wherein the at least one stable negative symptom associated with schizophrenia is stable for at least one month prior to the administration.
153. The use according to any one of Features 141 to 147, wherein there is not statistically significant improvement in any negative symptom associated with schizophrenia in the patient following the administration.
154. The use according to any one of Features 141 to 153, wherein the Compound (I) or a pharmaceutically acceptable salt thereof is administered in combination with at least one additional active agent.
155. The use according to Feature 154, wherein the at least one additional active agent treats at least one negative symptom associated with schizophrenia in the patient.
156. The use according to Feature 155, wherein the at least one negative symptom associated with schizophrenia is chosen from anhedonia, loss of motivation, and reduced interest in social interaction.
157. The use according to Feature 154, wherein the at least one additional therapeutic agent is chosen from aripiprazole, olanzapine, risperidone, and quetiapine fumarate.
158. The use according to any one of Features 141 to 157, wherein the Compound (I) or a pharmaceutically acceptable salt thereof is administered to the patient for more than 14 weeks.
159. The use according to any one of Features 141 to 158, wherein the Compound (I) or a pharmaceutically acceptable salt thereof is administered to the patient for more than 20 weeks.
160. The use according to any one of Features 141 to 159, wherein the patient is administered 1 mg to 100 mg of the Compound (I) or a pharmaceutically acceptable salt thereof once daily.
161. The use according to any one of Features 141 to 159, wherein the patient is administered 20 mg or 50 mg of the Compound (I) or a pharmaceutically acceptable salt thereof once daily.
162. The use according to any one of Features 141 to 159, wherein the patient is administered 20 mg of the Compound (I) or a pharmaceutically acceptable salt thereof once daily.
163. The use according to any one of Features 141 to 159, wherein the patient is administered 50 mg of the Compound (I) or a pharmaceutically acceptable salt thereof once daily.
164. The use according to any one of Features 141 to 163, wherein the administration does not treat at least one negative symptom associated with schizophrenia.
165. The use according to Feature 164, wherein the at least one negative symptom associated with schizophrenia is chosen from anhedonia, loss of motivation, and reduced interest in social interaction.
166. The use according to any one of Features 141 to 165, wherein the patient is on a stable regimen of psychotropic medications.
167. The use according to any one of Features 141 to 166, wherein the patient was diagnosed with schizophrenia at least one year prior to the administration.
168. The use according to any one of Features 141 to 167, wherein the patient has stable symptomatology for at least 3 months prior to the administration.
169. Use of Compound (I):

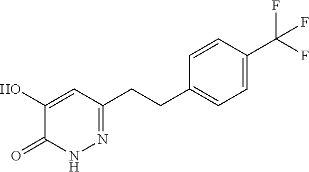 [SMILES: O=c1[nH]nc(CCc2ccc(C(F)(F)F)cc2)cc1O]

or a pharmaceutically acceptable salt thereof,
   in the manufacture of a medicament for use in a method of treating cognitive impairment in a patient in need thereof,
   the method comprising administering to the patient a therapeutically effective amount of the Compound (I) or a pharmaceutically acceptable salt thereof,
   wherein the patient exhibits at least one cognitive symptom prior to the administration.
170. The use according to Feature 169, wherein the method comprises:
   administering to the patient a therapeutically effective amount of the Compound (I) or a pharmaceutically acceptable salt thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient a therapeutically effective amount of the Compound (I) or a pharmaceutically acceptable salt thereof once daily if the 6 to 8 day administration improved at least one biomarker.
171. The use according to Feature 169 or 170, wherein the at least one cognitive symptom is chosen from impaired verbal memory, impaired working memory, impaired motor function, impaired attention, impaired processing speed, impaired verbal fluency, and impaired executive function.
172. The use according to any one of Features 169 to 171, wherein the at least one cognitive symptom is chosen from impaired attention, impaired memory, impaired reasoning, impaired problem solving, impaired working memory, impaired processing speed, impaired language functions, and impaired social cognition.
173. The use according to any one of Features 169 to 172, wherein the administration treats the at least one cognitive symptom.
174. The use according to any one of Features 169 to 173, wherein the at least one cognitive symptom is associated with a psychotic disorder.
175. The use according to Feature 174, wherein the psychotic disorder is chosen from psychosis, schizophreniform disorder, schizoaffective disorder, and schizophrenia unassociated with aggression.
176. The use according to Feature 174 or 175, wherein the psychotic disorder is schizophreniform disorder, schizoaffective disorder, and schizophrenia unassociated with aggression.
177. The use according to any one of Features 174 to 176, wherein the psychotic disorder is schizophrenia unassociated with aggression.
178. The use according to any one of Features 169 to 174, wherein the at least one cognitive symptom is associated with attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
179. The use according to any one of Features 169 to 174, wherein the at least one cognitive symptom is associated with Cushing's disease, Lewy body disease, multiple sclerosis, stroke, addictive disorder, pervasive development disorder, fragile X syndrome, Prader-Willi syndrome, and delirium.
180. The use according to any one of Features 169 to 179, wherein the patient is administered 50 mg of the Compound (I) or a pharmaceutically acceptable salt thereof once daily.
181. The use according to any one of Features 169 to 179, wherein the patient is administered 20 mg of the Compound (I) or a pharmaceutically acceptable salt thereof once daily.
182. The use according to any one of Features 169 to 181, wherein the Compound (I) or a pharmaceutically acceptable salt thereof is administered in combination with at least one additional active agent.
183. The use according to Feature 182, wherein the patient has schizophrenia and the at least one additional active agent treats at least one negative symptom in the patient.
184. The use according to Feature 183, wherein the at least one negative symptom is chosen from anhedonia, loss of motivation, and reduced interest in social interaction.
185. The use according to any one of Features 169 to 184, wherein the patient has at least one stable negative symptom chosen from anhedonia, loss of motivation, and reduced interest in social interaction prior to the administration.
186. The use according to any one of Features 141 to 185, wherein the Compound (I) or a pharmaceutically acceptable salt thereof is administered in the form of at least one film-coated tablet.
187. The use according to any one of Features 141 to 186, wherein the Compound (I) or a pharmaceutically acceptable salt thereof is administered with water or milk.
188. Use of Compound (I):

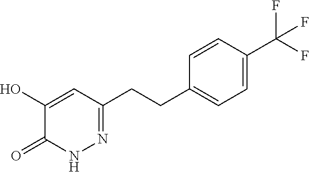 [SMILES: O=c1[nH]nc(CCc2ccc(C(F)(F)F)cc2)cc1O]

or a pharmaceutically acceptable salt thereof,
   in the manufacture of a medicament for use in a method of treating at least one cognitive symptom associated with schizophrenia in a patient,
   wherein the patient has no more than moderate-severe positive symptoms associated with schizophrenia (<5 rating on PANSS positive symptom items P1, P3, P4, P5, P6),
   the method comprising administering to the patient 1 mg to 500 mg of the Compound (I) or a pharmaceutically acceptable salt thereof once daily.
189. The use according to Feature 188, wherein the method comprises:
   administering to the patient 1 mg to 500 mg of the Compound (I) or a pharmaceutically acceptable salt thereof once daily for 6 to 8 days;
   determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
   continuing to administer to the patient 1 mg to 500 mg of the Compound (I) or a pharmaceutically acceptable salt thereof once daily if the 6 to 8 day administration improved at least one biomarker.
190. The use according to Feature 188 or 189, wherein the administration improves the patient's Brief Assessment of Cognition in Schizophrenia (BACS) Composite Score relative to a BACS Composite Score measured for the patient prior to the administration.
191. The use according to any one of Features 188 to 190, wherein the administration improves the patient's Schizophrenia Cognition Rating Scale (SCORS) interviewer total score relative to a SCORS interviewer total score measured for the patient prior to the administration.
192. The use according to any one of Features 188 to 191, wherein the administration improves at least one metric chosen from:
   the patient's performance on the Continuous Performance Test-Identical Pairs (CPT-IP) test relative to a CPT-IP test prior to the administration;
   the patient's performance on the Brief Visuospatial Memory Test-Revised (BVMT-R) test relative to the patient's performance on a BVMT-R test prior to the administration;
   the patient's performance on the Mayer-Salovey-Caruso Emotional Intelligence Test (MSCEIT) relative to the patient's performance on a MSCEIT prior to the administration;
   the patient's performance on the Virtual Reality Functional Capacity Assessment Tool (VRFCAT) relative to the patient's performance on a VRFCAT prior to the administration; and
   the patient's Clinical Global Impression-Severity Scale (CGI-S) score relative to a CGI-S score measured for the patient prior to the administration.
193. The use according to any one of Features 188 to 192, wherein there is not statistically significant improvement in any negative symptom associated with schizophrenia in the patient following the administration.
194. The use according to any one of Features 188 to 193, wherein the administration does not treat at least one negative symptom associated with schizophrenia.
195. The use according to Feature 194, wherein the at least one negative symptom associated with schizophrenia is chosen from anhedonia, loss of motivation, and reduced interest in social interaction.
196. The use according to any one of Features 188 to 195, wherein the patient is administered 50 mg of the Compound (I) or a pharmaceutically acceptable salt thereof once daily.
197. The use according to any one of Features 188 to 195, wherein the patient is administered 20 mg of the Compound (I) or a pharmaceutically acceptable salt thereof once daily.
198. Use of Compound (I):

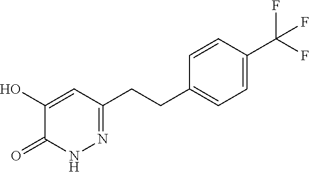 [SMILES: O=c1[nH]nc(CCc2ccc(C(F)(F)F)cc2)cc1O]

or a pharmaceutically acceptable salt thereof,
   in the manufacture of a medicament for use in a method of increasing D-serine levels, long-term potentiation, and/or synaptic plasticity in a patient in need thereof,
   the method comprising administering to the patient less than 500 mg of the Compound (I) or a pharmaceutically acceptable salt thereof once daily.
199. The use according to Feature 198, wherein the patient is administered 50 mg of the Compound (I) or a pharmaceutically acceptable salt thereof once daily.
200. The use according to Feature 198, wherein the patient is administered 20 mg of the Compound (I) or a pharmaceutically acceptable salt thereof once daily.
201. The use according to any one of Features 198 to 200, wherein the method increases D-serine levels in the patient.
202. The use according to any one of Features 198 to 201, wherein the method increases long-term potentiation in the patient.
203. The use according to any one of Features 198 to 202, wherein the method increases synaptic plasticity in the patient.
204. The use according to any one of Features 198 to 203, wherein the patient exhibits at least one cognitive symptom prior to the administration.
205. The use according to Feature 204, wherein the at least one cognitive symptom is chosen from impaired verbal memory, impaired working memory, impaired motor function, impaired attention, impaired processing speed, impaired verbal fluency, and impaired executive function.
206. The use according to Feature 204, wherein the at least one cognitive symptom is chosen from impaired attention, impaired memory, impaired reasoning, impaired problem solving, impaired working memory, impaired processing speed, impaired language functions, and impaired social cognition.
207. The use according to any one of Features 198 to 206, wherein the administration improves at least one biomarker chosen from the patient's eye-blink conditioning (EBC) response, the patient's mismatch negativity (MMN) amplitude, and the patient's auditory steady state response (ASSR) gamma band power.
208. The use according to any one of Features 198 to 207, wherein the patient suffers from a disease chosen from attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.
209. The use according to any one of Features 198 to 207, wherein the patient suffers from a disease chosen from psychosis, schizophreniform disorder, schizoaffective disorder, and schizophrenia unassociated with aggression.
210. The use according to any one of Features 198 to 207, wherein the patient has no more than moderate-severe positive symptoms associated with schizophrenia (<5 rating on PANSS positive symptom items P1, P3, P4, P5, P6).

### BRIEF DESCRIPTION OF THE DRAWINGS

FIG. 1A is a graph depicting the results of D-serine administration on short-term potentiation in the rat hippocampus compared to vehicle.

FIG. 1B is a graph depicting the results of D-serine administration on long-term depression in the rat hippocampus compared to vehicle.

FIG. 1C is a chart showing changes in potentiation and depression with D-serine administration compared to vehicle.

FIG. 2 depicts the effects of Compound (I) on mouse hippocampal synaptic plasticity after a single oral dose of Compound (I).

FIG. 3 depicts the effects of Compound (I) on mouse hippocampal synaptic plasticity after sub-chronic (14-day) oral doses of Compound (I).

FIG. 4A is a chart depicting plasma D-serine levels after sub-chronic dosing with Compound (I) in a mouse model.

FIG. 4B is a chart depicting cerebellum D-serine levels after sub-chronic dosing with Compound (I) in a mouse model.

FIG. 5 depicts the study design for the Phase 2 INTERACT study of Compound (I) as an adjunctive therapy in adults with schizophrenia with persistent negative symptoms (ClinicalTrials.gov identifier: NCT03382639).

FIG. 6 is a graph depicting the least-squares (LS) mean change from baseline in PANSS NSFS during the 12-week treatment period for patients enrolled in the INTERACT study.

FIG. 7 is a graph depicting the least-squares (LS) mean change from baseline in BACS composite score during the 12-week treatment period for patients enrolled in the INTERACT study.

FIG. 8 is a chart depicting the least-squares (LS) mean change from baseline in the SCORS interviewer total score during the 12-week treatment period for patients enrolled in the INTERACT study.

FIG. 9 depicts the study design for a Phase 1b study designed to evaluate pharmacodynamic effects, safety, tolerability and pharmacokinetics of multiple oral doses of Compound (I) in patients with schizophrenia (ClinicalTrials.gov identifier: NCT03359785).

FIG. 10 depicts the eyeblink conditioning assessment used in a Phase 1b study evaluating multiple oral doses of Compound (I) in patients with schizophrenia (ClinicalTrials.gov identifier: NCT03359785).

FIGS. 11A and 11B are graphs depicting mismatch negativity (MMN) at baseline (pre-drug and pre-placebo).

FIG. 12A is a chart showing least-squares mean changes in eyeblink conditioning following treatment with placebo or Compound (I) 50 mg in patients with schizophrenia.

FIG. 12B is a chart showing least-squares mean changes in eyeblink conditioning following treatment with placebo or Compound (I) 500 mg in patients with schizophrenia.

FIG. 13A is a chart showing least-squares mean changes in MMN following treatment with placebo or Compound (I) 50 mg in patients with schizophrenia.

FIG. 13B is a chart showing least-squares mean changes in MMN following treatment with placebo or Compound (I) 500 mg in patients with schizophrenia.

FIG. 14A is a chart showing least-squares mean changes in ASSR following treatment with placebo or Compound (I) 50 mg in patients with schizophrenia.

FIG. 14B is a chart showing least-squares mean changes in ASSR following treatment with placebo or Compound (I) 500 mg in patients with schizophrenia.

FIG. 15 summarizes the directionality of pharmacodynamic endpoint changes observed in a Phase 1b study evaluating multiple oral doses of Compound (I) in patients with schizophrenia (ClinicalTrials.gov identifier: NCT03359785).

FIGS. 16A and 16B are study design schematics for a planned Phase 2b study evaluating the safety and efficacy of Compound (I) compared with placebo on improving cognitive performance in subjects with schizophrenia.

### DEFINITIONS

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

As used herein, the term “active pharmaceutical ingredient” (“API”), “active agent,” or “therapeutic agent” refers to a biologically active compound.

As used herein, “administration” of an API to a patient refers to any route (e.g., oral delivery) of introducing or delivering the API to the patient. Administration includes self-administration and the administration by another.

As used herein, a “condition,” “disorder,” or “disease” relates to any unhealthy or abnormal state.

As used herein, an “effective amount,” “effective dose,” or a “therapeutically effective amount” refers to an amount of a molecule that treats, upon single or multiple dose administration, a patient suffering from a condition. An effective amount can be determined by the attending diagnostician through the use of known techniques and by observing results obtained under analogous circumstances. In determining the effective amount, a number of factors are considered by the attending diagnostician, including, but not limited to: the species of patient; its size, age, and general health; the specific condition, disorder, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances.

As used herein, an amount expressed in terms of “mg of at least one compound chosen from Compound (I) and pharmaceutically acceptable salts thereof” is based on the total weight of the free base of Compound (I) present, in the form of the free base and/or one or more pharmaceutically acceptable salts of Compound (I).

As used herein, a “mammal” refers to domesticated animals (e.g., dogs, cats, and horses) and humans. In some embodiments, the mammal is a human.

As used herein, the term “modulate” refers to altering positively or negatively. Non-limiting example modulations include a 1% change, a 2% change, a 5% change, a 10% change, a 25% change, a 50% change, a 75% change, or a 100% change.

As used herein, the terms “patient” and “subject” are used interchangeably and refer to a mammal, such as, e.g., a human.

As used herein, a “pharmaceutically acceptable excipient” refers to a carrier or an excipient that is useful in preparing a pharmaceutical composition. For example, a pharmaceutically acceptable excipient is generally safe and includes carriers and excipients that are generally considered acceptable for mammalian pharmaceutical use. As a non-limiting example, pharmaceutically acceptable excipients may be solid, semi-solid, or liquid materials which in the aggregate can serve as a vehicle or medium for the active ingredient. Some examples of pharmaceutically acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

As used herein, the term “pharmaceutically acceptable salt” refers to a non-toxic salt form of a compound of this disclosure. Pharmaceutically acceptable salts of Compound (I) of this disclosure include those derived from suitable inorganic and organic acids and bases. Pharmaceutically acceptable salts are well known in the art. Suitable pharmaceutically acceptable salts are, e.g., those disclosed in Berge, S. M., et al. J. Pharma. Sci. 66:1-19 (1977). Non-limiting examples of pharmaceutically acceptable salts disclosed in that article include: acetate; benzenesulfonate; benzoate; bicarbonate; bitartrate; bromide; calcium edetate; camsylate; carbonate; chloride; citrate; dihydrochloride; edetate; edisylate; estolate; esylate; fumarate; gluceptate; gluconate; glutamate; glycollylarsanilate; hexylresorcinate; hydrabamine; hydrobromide; hydrochloride; hydroxynaphthoate; iodide; isethionate; lactate; lactobionate; malate; maleate; mandelate; mesylate; methylbromide; methylnitrate; methylsulfate; mucate; napsylate; nitrate; pamoate (embonate); pantothenate; phosphate/diphosphate; polygalacturonate; salicylate; stearate; subacetate; succinate; sulfate; tannate; tartrate; teociate; triethiodide; benzathine; chloroprocaine; choline; diethanolamine; ethylenediamine; meglumine; procaine; aluminum; calcium; lithium; magnesium; potassium; sodium; and zinc.

Non-limiting examples of pharmaceutically acceptable salts derived from appropriate acids include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Additional non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Non-limiting examples of pharmaceutically acceptable salts derived from appropriate bases include alkali metal, alkaline earth metal, ammonium, and N⁺ (C₁₋₄ alkyl)₄ salts. This disclosure also envisions the quaternization of any basic nitrogen-containing groups of the compounds disclosed herein. Non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other non-limiting examples of pharmaceutically acceptable salts include besylate and glucosamine salts.

Additionally, “at least one compound chosen from Compound (I) and pharmaceutically acceptable salts” thereof may be used interchangeably herein with “at least one compound or salt chosen from Compound (I) and pharmaceutically acceptable salts” and “at least one entity chosen from Compound (I) and pharmaceutically acceptable salts.” Similarly, “the at least one compound” may be used interchangeably herein with “the at least one compound or salt” or “the at least one entity.”

As used herein, the term “reduce” refers to altering negatively by at least 5% including, but not limited to, altering negatively by 5%, altering negatively by 10%, altering negatively by 25%, altering negatively by 30%, altering negatively by 50%, altering negatively by 75%, or altering negatively by 100%.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art. As a non-limiting example, in some embodiments, administration effects may be assessed using a cognitive battery (e.g., BACS, MCCB, CogState, or Cambridge Cognition) or a measure of functional capacity (e.g., SCORS, UPSA, UPSA-B, CAI, or VRFCAT).

As used herein, the “Calgary Depression Scale for Schizophrenia” or “CDSS” is used to assess symptoms of major depressive disorder in patients with schizophrenia, and specifically designed to assess comorbid depressive symptoms. See, e.g., Addington D, Addington J, Maticka-Tyndale E. “Assessing depression in schizophrenia: the Calgary Depression Scale.” Br. J. Psychiatry Suppl. 1993; (22): 39-44. The CDSS consists of 9 items: depressed mood, hopelessness, self-deprecation, guilty ideas of reference, pathological guilt, depression worse in the morning, early wakening, suicide, and observed depression. Each item is rated using a 0 to 3 point scale (0-3); the CDSS score can range from 0 to 27. The items on the CDSS are all typical depressive symptoms and do not appear to overlap with the negative symptoms of schizophrenia. The CDSS will be conducted by the investigator or other qualified site personnel.

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Furthermore, the disclosure encompasses all variations, combinations, and permutations in which at least one limitation, element, clause, and descriptive term from at least one of the listed claims is introduced into another claim. For example, any claim that is dependent on another claim can be modified to include at least one limitation found in any other claim that is dependent on the same base claim. Where elements are presented as lists, such as, e.g., in Markush group format, each subgroup of the elements is also disclosed, and any element(s) can be removed from the group. It should be understood that, in general, where the disclosure, or aspects of the disclosure, is/are referred to as comprising particular elements and/or features, embodiments of the disclosure or aspects of the disclosure consist, or consist essentially of, such elements and/or features. For purposes of simplicity, those embodiments have not been specifically set forth in haec verba herein. Where ranges are given (such as, e.g., from [X] to [Y]), endpoints (such as, e.g., [X] and [Y] in the phrase “from [X] to [Y]”) are included unless otherwise indicated. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those of ordinary skill in the art will recognize or be able to ascertain using no more than routine experimentation, many equivalents to the specific embodiments of the disclosure described herein. Such equivalents are intended to be encompassed by the following claims.

### EXAMPLES

The following examples are intended to be illustrative and are not meant in any way to limit the scope of the disclosure.

### Abbreviations

% Percent
aCSF Artificial cerebrospinal fluid
ANCOVA Analysis of covariance
ANOVA Analysis of variance
ASSR Auditory Steady State Response
ASST Attention set-shifting task
BACS Brief Assessment of Cognition in Schizophrenia
BNSS Brief Negative Symptom Scale
CI Confidence interval
CIAS Cognitive impairment associated with schizophrenia
CR Conditioned response
CS Conditioned stimulus
CSF Cerebrospinal fluid
d Day(s)
DAAO D-amino acid oxidase
DB Double-bind
EBC Eyeblink conditioning
ECG Electrocardiogram
EEG Electroencephalogram
EPSP Excitatory postsynaptic potential
fEPSP Field excitatory postsynaptic potential
F/U Follow-up
g Gram
h Hour(s)
HFS High-frequency stimulation
Hz Hertz
kg Kilogram(s)
LS Least-squares
LTD Long-term depression
LTP Long-term potentiation
m² Square meter(s)
mg Milligram
min Minute(s)
mL Milliliter(s)
mm Millimeter(s)
MMN Mismatch negativity
NMDA N-methyl-d-aspartate
ns Not significant
PANSS NSFS Positive and Negative Syndrome Scale-Negative Symptom Factor
Score
PK Pharmacokinetics
PD Pharmacodynamics
qd Once daily
s Second(s)
SCORS Schizophrenia Cognition Rating Scale
SD Standard deviation
SE Standard error
STP Short-term potentiation
TEAE Treatment-emergent adverse event
US Unconditioned stimulus
UR Unconditioned response

### Example 1: Pre-Clinical Evaluation of Hippocampal Long-Term Potentiation after Acute and Sub-Chronic Oral Dosing with Compound (I) in Mice

DAAO inhibition increases levels of D-serine, a co-agonist of the N-methyl-d-aspartate (NMDA) receptor. The DAAO enzyme is highly expressed in the cerebellum and, as such, inhibition of DAAO by Compound (I) should result in increases in D-serine levels in the cerebellum, which can then also be measured in the cerebrospinal fluid (CSF). Increases in cerebellum D-serine levels may also influence distal regions of the brain (such as the hippocampus) or cortical regions, through a mechanism that remains to be elucidated.

Preclinical studies in rodents have explored the effects of Compound (I) on cognitive performance tasks (Table 1). In Table 1, a + sign means efficacious, a − sign means not efficacious, and an empty cell indicates a dose was not tested. In these preclinical studies, sub-chronic dosing with Compound (I) over 14 days led to sensitization, compared with acute dosing, in the behavioral response for the novel object recognition task (i.e., decreased minimum effective dose with loss of efficacy at higher doses). These findings suggest that Compound (I) may influence hippocampal synaptic plasticity after sub-chronic dosing.

TABLE 1
Sensitization Response in In Vivo Models After Sub-Chronic Dosing of Compound (I)

| Efficacy/ | | | | | | | | | | | |
|---|---|---|---|---|---|---|---|---|---|---|---|
| pharmacodynamic | | Dose (mg/kg) | | | | | | | | | |
| marker | Species | 0.001 | 0.003 | 0.01 | 0.03 | 0.1 | 0.3 | 1 | 3 | 10 | 30 |

Single Dose

| | | | | | | | |
|---|---|---|---|---|---|---|---|
| Novel object | Mouse | − | + | + | + | + | |
| recognition | | | | | | | |
| (cognition) | | | | | | | |
| ASST (executive | Rat | | | +/− | + | + | + |
| function) | | | | | | | |
| BALB/C social | Mouse | | − | + | + | | |
| interaction (acute) | | | | | | | |
| Poly I:C social | Mouse | | − | − | + | | |
| interaction (acute) | | | | | | | |
| D-serine increase | Mouse | | + | + | + | + | |
| in cerebellum | | | | | | | |
| (6 hours) | | | | | | | |

10-Day Once Daily (QD)

| | | | |
|---|---|---|---|
| Eyeblink | Mouse | + | + |
| conditioning | | | |
| (ataxia and | | | |
| schizophrenia) | | | |

14-Day Once Daily (QD)

| | | | | | | | | | | |
|---|---|---|---|---|---|---|---|---|---|---|
| Novel object | Mouse | − | + | + | + | + | − | − | − | |
| recognition | | | | | | | | | | |
| (cognition) | | | | | | | | | | |
| Attention | Rat | | | +/− | | + | | + | | + |
| set-shifting task | | | | | | | | | | |
| (ASST) | | | | | | | | | | |
| (executive | | | | | | | | | | |
| function) | | | | | | | | | | |
| BALB/C social | Mouse | − | − | + | + | + | + | + | + | |
| interaction | | | | | | | | | | |
| (negative | | | | | | | | | | |
| symptoms) | | | | | | | | | | |
| D-serine increase | Mouse | | | | − | − | + | + | + | |
| in cerebellum | | | | | | | | | | |
| (6 hours) | | | | | | | | | | |

In this study, hippocampal synaptic plasticity was evaluated using a well-established long-term potentiation (LTP) paradigm after acute and sub-chronic (14 days) dosing with Compound (I).

### Preparation of Rat Hippocampal Slices

To obtain rat hippocampal slices, 4-week-old Sprague Dawley rats (provided by Elevage Janvier, Le Genest-Saint-Isle, France) were euthanized by fast decapitation without anesthesia. The brain was quickly removed and soaked in ice-cold oxygenated buffer: 2 mM KCl, 1.2 mM NaH₂PO₄, 7 mM MgCl₂, 0.5 mM CaCl₂), 26 mM NaHCO₃, 11 mM glucose, and 250 mM sucrose. Hippocampal slices were prepared at 400 μm thickness with a McILWAIN tissue chopper. Slices were allowed to recover at room temperature for at least 1 hour in artificial CSF (aCSF): 126 mM NaCl, 3.5 mM KCl, 1.2 mM NaH₂PO₄, 1.3 mM MgCl₂, 2 mM CaCl₂), 25 mM NaHCO₃, and 11 mM glucose. Slices were continuously perfused with oxygenated aCSF during experiments. The animals were housed and used in accordance with French and European legislation.

### Preparation of Mouse Hippocampal Slices

Wild-type mice aged 5-6 weeks (total of 97; n=5-8 per condition) were given acute or sub-chronic (14-day) oral doses of Compound (I) (from 0.001 mg/kg to 10 mg/kg, p.o.) or vehicle (1% Tween 80 in 0.5% methylcellulose). Hippocampal slices were harvested after 5 hours. Cerebellum and blood plasma were also obtained to measure D-serine levels achieved in the experimental animals.

Electrophysiological recordings were obtained from a single hippocampal slice placed on the chamber and perfused with aCSF at a constant rate. Extracellular field excitatory postsynaptic potentials (fEPSPs) were recorded in the CA1 stratum radiatum using a glass micropipette filled with aCSF. fEPSPs were evoked by electrical stimulation of Schaffer collaterals/commissural pathway at 0.1 Hz with a glass stimulating electrode placed in the stratum radiatum.

To test the effect of Compound (I) on basal synaptic transmission, Input/Output (I/V) curves were constructed at the beginning of the experiment. The slope of fEPSPs was measured and plotted against different intensities of stimulation (from 0 to 100 μA). Long-term potentiation (LTP) was induced by a theta burst stimulation protocol (10 bursts of 4 pulses at 100 Hz, with 200 ms between bursts) at baseline stimulation intensity. Following this conditioning stimulus, a 1-hour test period was recorded where responses were again elicited by a single stimulation every 10 seconds (0.1 Hz) at the same stimulus intensity.

### Results

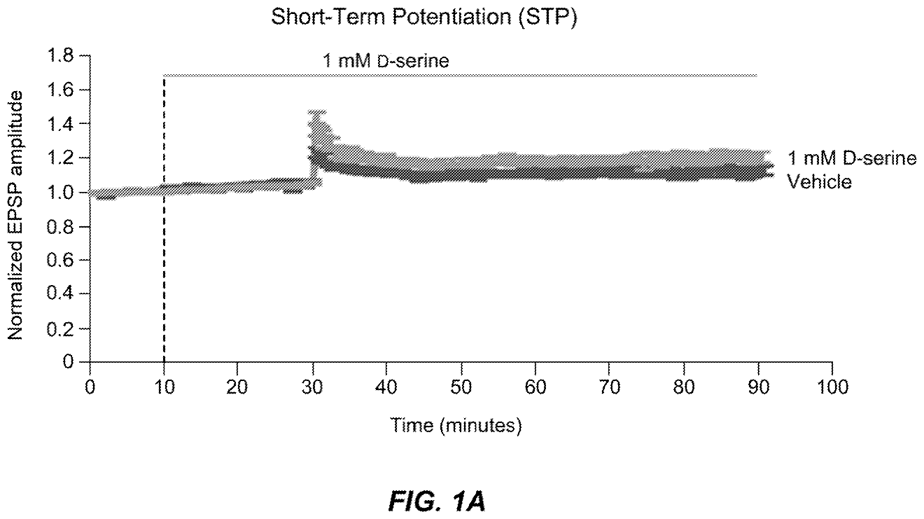

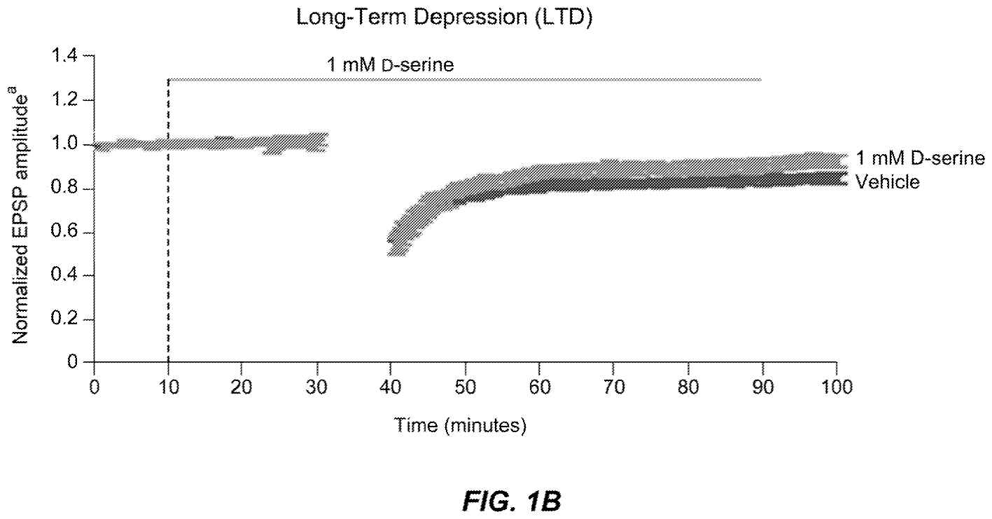

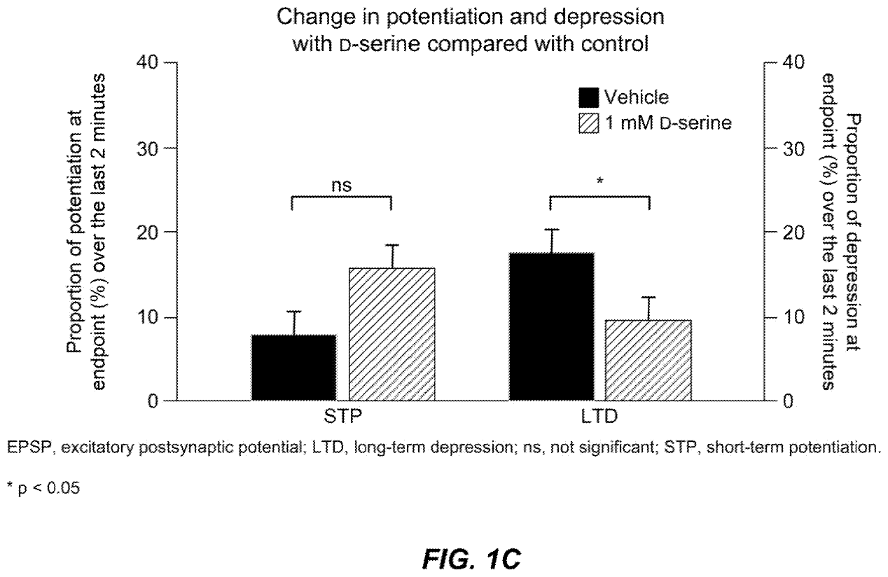

Acute high doses (1 mM) of D-serine induced hippocampal synaptic plasticity (short-term potentiation and long-term depression) compared with controls in the rat hippocampus (FIGS. 1A-1C). Data in FIGS. 1A and 1B were normalized to the 10-minute control period. D-serine increased the potentiation of excitatory postsynaptic potential (EPSP) amplitudes, although this change was not statistically significant (p=0.0603, unpaired t-test). However, D-serine significantly decreased the depression of EPSP amplitudes (p=0.0374, unpaired t-test).

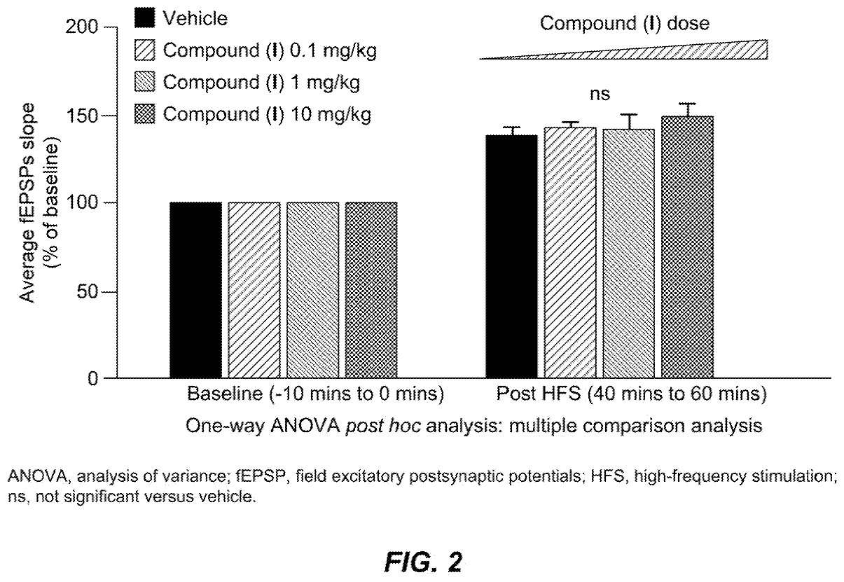

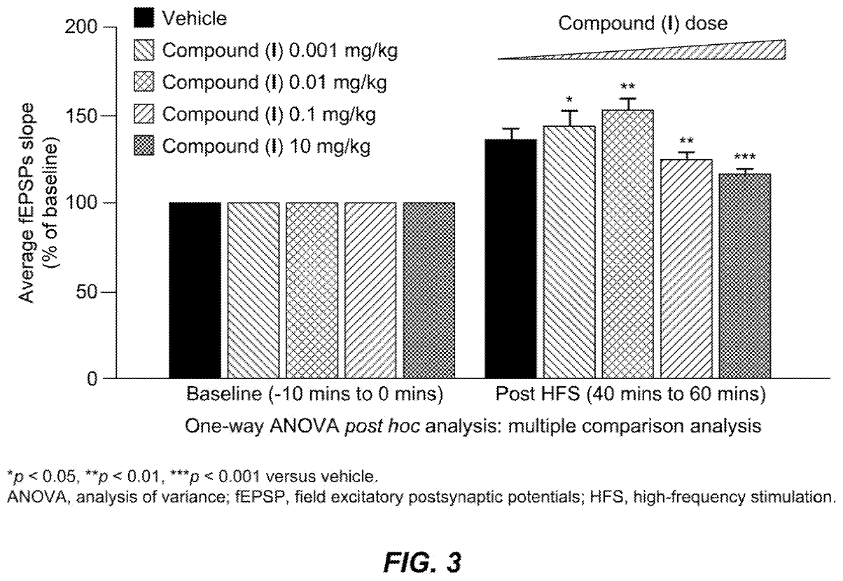

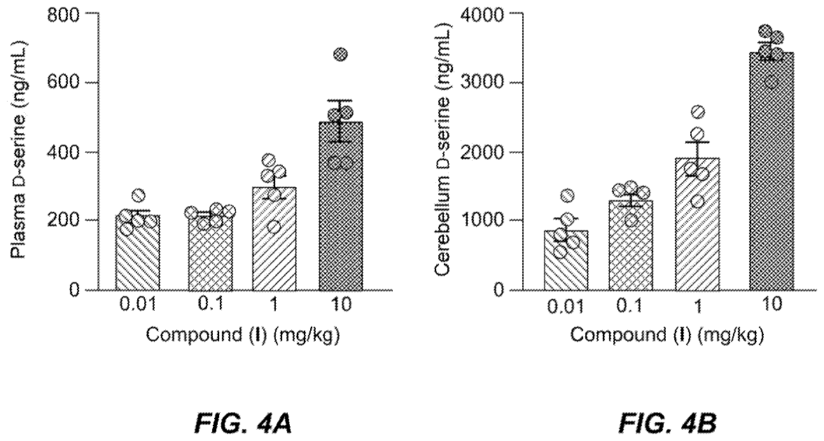

Single oral doses of Compound (I) did not significantly affect LTP (based on one-way analysis of variance [ANOVA] vs. administration of vehicle in the mouse hippocampus) (FIG. 2). By contrast, low sub-chronic oral doses of Compound (I) (0.001 mg/kg and 0.01 mg/kg) induced increased LTP, whereas higher doses (0.1 mg/kg and 10 mg/kg) were associated with decreased LTP (all doses p<0.05 vs vehicle based on a one-way ANOVA post-hoc multiple comparison analysis) (FIG. 3), suggesting increases in synaptic plasticity. In addition, dose-dependent increases in plasma and cerebellum D-serine levels were observed after sub-chronic dosing of mice with Compound (I) (FIGS. 4A, 4B). Specifically, Compound (I) produced a dose-dependent increase in D-serine levels in the mouse brain (from 8.7 nmol/g to 34 nmol/g) and mouse plasma (from 213 ng/ml to 490 ng/ml).

### Discussion

Compound (I) induced sensitization of in vivo responses when dosed chronically, which suggested potential changes in synaptic plasticity. Acute dosing of Compound (I) did not change LTP, which is a phenomenon that reflects changes in neuronal synaptic plasticity. Acute changes in D-serine levels were not likely to produce a structural change in the synapse; however, plasticity changes were observed after chronic increases in D-serine levels, which can be achieved after chronic dosing with Compound (I). Sub-chronic dosing with lower doses of Compound (I) significantly increased LTP, suggesting increases in synaptic plasticity. Higher doses (0.1 mg/kg and 10 mg/kg) induced decreases in LTP, indicating an inverted U-shaped dose response. This phenomenon may underlie the leftward shift in behavioral responses (i.e., novel object recognition task) observed after acute vs chronic dosing. The leftward shift of the dose-response relationship reported in this study suggests low doses may be efficacious in clinical trials of DAAO inhibitors such as Compound (I).

### Example 2: 10 mg Formulation Comprising Compound (I) (Preparation 1)

Hydroxypropyl cellulose (810 g, grade L, produced by NIPPON SODA CO., LTD.) was dissolved in purified water (12690 g) to prepare a binder solution. Compound (I) (900 g), D-mannitol (19620 g, PEARLITOL 50C, produced by Roquette Co.), and microcrystalline cellulose (2700 g, CEOLUS PH-101, produced by Asahi Kasei Corporation) were granulated in a fluid-bed granulator (FD-WGS-30, manufactured by Powrex Co.) while spraying a binder solution and then dried to obtain a granulated powder. The granulated powder was manufactured for 2 batches. The granulated powder was then milled, 44060 g of the obtained milled powder was weighed, and low-substituted hydroxypropyl cellulose (4950 g, grade LH-21, produced by Shin-Etsu Chemical Co., Ltd.) (containing 11% hydroxypropoxy group (dry weight)), and magnesium stearate (495 g, produced by Taihei Chemical Industrial Co., Ltd.) were added and mixed to obtain a mixed powder. The obtained mixed powder was made into tablets using a rotary tablet press (AQUARIUS 08242L2JI, manufactured by KIKUSUI SEISAKUSHO LTD.) to obtain uncoated tablets having a weight of 300 mg per tablet and a diameter of 9 mm. 40500 g of the obtained uncoated tablets were inserted into a Doria coater (DRC-900S, manufactured by Powrex Co.), and an aqueous dispersion of hypromellose, macrogol 6000, titanium oxide, red ferric oxide, and yellow ferric oxide was sprayed to prepare 12.2 mg of a film coating per tablet and obtain Preparation 1 (film-coated tablets). The composition of Preparation 1 (on a per tablet basis) is shown in Table 2.

TABLE 2
Composition of Preparation 1 per Tablet (10 mg Formulation)

| | Amount |
|---|---|
| Component | (mg) |
| Compound (I) | 10 |
| D-mannitol (PEARLITOL 50C) | 218 |
| Microcrystalline Cellulose (CEOLUS PH-101) | 30 |
| Hydroxypropyl Cellulose (HPC-L) | 9 |
| Low-substituted Hydroxypropyl Cellulose (LH-21) | 30 |
| Magnesium Stearate | 3 |
| Hypromellose (TC-5R) | 9 |
| Titanium Dioxide | 1 |
| Red Ferric Oxide | 0.1 |
| Yellow Ferric Oxide | 0.1 |
| Total | 310.2 |

### Example 3: 25 mg Formulation Comprising Compound (I) (Preparation 2)

Hydroxypropyl cellulose (810 g, grade L, produced by NIPPON SODA CO., LTD.) was dissolved in purified water (12690 g) to prepare a binder solution. Compound (A) (2250 g), D-mannitol (18270 g, PEARLITOL 50C, produced by Roquette Co.), and microcrystalline cellulose (2700 g, CEOLUS PH-101, produced by Asahi Kasei Corporation) were granulated in a fluid-bed granulator (FD-WGS-30, manufactured by Powrex Co.) while spraying a binder solution and then dried to obtain a granulated powder. The granulated powder was manufactured for 2 batches. The granulated powder was then milled, 44060 g of the obtained milled powder was weighed, and low-substituted hydroxypropyl cellulose (4950 g, grade LH-21, produced by Shin-Etsu Chemical Co., Ltd.) (containing 11% hydroxypropoxy group (dry weight)), and magnesium stearate (495 g, produced by Taihei Chemical Industrial Co., Ltd.) were added and mixed to obtain a mixed powder. The obtained mixed powder was made into tablets using a rotary tablet press (AQUARIUS 08242L2JI, manufactured by KIKUSUI SEISAKUSHO LTD.) to obtain uncoated tablets having a weight of 300 mg per tablet and a diameter of 9 mm. 40500 g of the obtained uncoated tablets were inserted into a Doria coater (DRC-900S, manufactured by Powrex Co.), and an aqueous dispersion of hypromellose, macrogol 6000, titanium oxide, red ferric oxide, and yellow ferric oxide was sprayed to prepare 12.2 mg of a film coating per tablet and obtain Preparation 2 (film-coated tablets). The composition of Preparation 2 (on a per tablet basis) is shown in Table 3.

TABLE 3
Composition of Preparation 2 per Tablet (25 mg Formulation)

| Component | Amount (mg) |
|---|---|
| Compound I | 25 |
| D-mannitol (PEARLITOL 50C) | 203 |
| Microcrystalline Cellulose (CEOLUS PH-101) | 30 |
| Hydroxypropyl Cellulose (HPC-L) | 9 |
| Low-substituted Hydroxypropyl Cellulose (LH-21) | 30 |
| Magnesium Stearate | 3 |
| Hypromellose (TC-5R) | 9 |
| Titanium Dioxide | 1 |
| Red Ferric Oxide | 0.1 |
| Yellow Ferric Oxide | 0.1 |
| Total | 310.2 |

### Example 4: 100 mg Formulation Comprising Compound (I) (Preparation 3)

Preparation 3 (film-coated tablets), which contains 100 mg of Compound (I) per tablet, can be produced by similar production methods. The composition of Preparation 3 (on a per tablet basis) is shown in Table 4.

TABLE 4
Composition of Preparation 3 per Tablet

| Component | Amount (mg) |
|---|---|
| Compound (A) | 100 |
| D-mannitol (PEARLITOL 50C) | 128 |
| Microcrystalline Cellulose (CEOLUS PH-101) | 30 |
| Hydroxypropyl Cellulose (HPC-L) | 9 |
| Low-substituted Hydroxypropyl Cellulose (LH-21) | 30 |
| Magnesium Stearate | 3 |
| Hypromellose (TC-5R) | 9 |
| Titanium Dioxide | 1 |
| Red Ferric Oxide | 0.1 |
| Yellow Ferric Oxide | 0.1 |
| Total | 310.2 |

### Example 5: 50 mg Formulation Comprising Compound (I) (Preparation 4)

Preparation 4 (film-coated tablets), which contains 50 mg of Compound (I) per tablet, can be produced by similar production methods. The composition of Preparation 4 (on a per tablet basis) is shown in Table 5.

TABLE 5
Composition of Preparation 4 per Tablet

| | Amount |
|---|---|
| Component | (mg) |
| Compound (I) | 50 |
| D-mannitol (PEARLITOL 50C) | 178 |
| Microcrystalline Cellulose (CEOLUS PH-101) | 30 |
| Hydroxypropyl Cellulose (HPC-L) | 9 |
| Low-substituted Hydroxypropyl Cellulose (LH-21) | 30 |
| Magnesium Stearate | 3 |
| Hypromellose (TC-5R) | 9 |
| Titanium Dioxide | 1 |
| Red Ferric Oxide | 0.1 |
| Yellow Ferric Oxide | 0.1 |
| Total | 310.2 |

### Example 6: A Study to Evaluate Efficacy, Safety, Tolerability, and Pharmacokinetics of 3 Dose Levels of Compound (I) in Adjunctive Treatment of Adult Participants with Negative Symptoms of Schizophrenia (NCT03382639)

A Phase 2 randomized, double-blind, parallel-group, placebo-controlled, dose-range finding study clinical trial (INTERACT) was performed to determine whether add-on/adjunctive Compound (I) is superior to placebo in the treatment of adult patients with persistent negative symptoms of schizophrenia (ClinicalTrials.gov Identifier: NCT03382639). This multi-center trial was conducted in the United States, Bulgaria, Czech Republic, Italy, Poland, the Russian Federation, Serbia, Spain, and Ukraine. The study enrolled 256 of the 503 patients who were screened for eligibility (Tables 5, 6). Eligible participants were aged 18-60 years with symptomatically stable schizophrenia and had a baseline Brief Negative Symptom Scale (BNSS) score of 28 or higher (12-item, excluding item 4). Additionally, eligible participants were receiving stable antipsychotic treatment at a 2-6 mg daily dose of risperidone equivalents, and were allowed to be treated with a second antipsychotic if it was used as a hypnotic at a subtherapeutic dose (subject to sponsor approval). The most common concomitant antipsychotic medications were aripiprazole (25.4%), olanzapine (18.8%), risperidone (18.0%) and quetiapine fumarate (9.4%). To overcome potential confounding effects, those with depressive and extrapyramidal symptoms were excluded.

Participants were randomly assigned (by chance, e.g., flipping a coin) to one of the four treatment groups in the double-blind period: (1) Compound (I) 50 mg once daily (5 Preparation 1 tablets); (2) Compound (I) 125 mg once daily (5 Preparation 2 tablets); (3) Compound (I) 500 mg once daily (5 Preparation 3 tablets); and (4) placebo once daily (5 oral placebo tablets). Dose selection for this study was based on target occupancy and elevation of D-serine.

Overall, the 256 participants were randomized 3:2:2:2 to receive placebo, Compound (I) 50 mg, Compound (I) 125 mg, and Compound (I) 500 mg, respectively, with 228 (89.1%) participants completing the study. Participants' mean age was 40 years (range 18-60 years), 168 (65.6%) were male, and 208 (81.3%) were white. Demographic and baseline characteristics were evenly distributed across treatment groups.

Compound (I) was orally administered in the form of a tablet once daily for up to 14 weeks. Compound (I) placebo-matching tablets were similarly orally administered once daily for up to 14 weeks. The treatment group remained undisclosed to the participant and study doctor/site personnel during the study unless there was an urgent medical need.

Inclusion criteria for the study included:
1. Has a current diagnosis of schizophrenia as defined by the Mini International Neuropsychiatric Interview (MINI) 7.0.2 for Psychotic Disorders for the Diagnostic and Statistical Manual of Mental Disorders, Fifth Edition (DSM-5) and the general psychiatric evaluation.
2. Initial diagnosis must be greater than or equal to (>=1) year from screening.
3. Is receiving primary background antipsychotic therapy (other than clozapine) at a total daily dose between 2 and 6 mg of risperidone equivalents. Concomitant treatment with a sub-therapeutic dose of a second antipsychotic may be permitted with sponsor or designee approval if used to treat specific symptoms, such as insomnia or anxiety (for example, quetiapine 25-50 mg or its equivalent as needed for anxiety), but not if it is used for refractory positive psychosis symptoms.
4. Is treated with a stable regimen of psychotropic medications with no clinically meaningful change (no increase in dose, less than or equal to [<=] 25 percent [%] decrease in dose for tolerability) in the prescribed dose for 2 months before the screening visit and no dose adjustment is anticipated throughout study participation up to the Day 84/early termination visit.
5. Has a BNSS total score (12-item, excluding number 4)>=28; stable Single-blind Placebo Run-in and baseline BNSS total (12-item, excluding number 4) scores (<=20% change from the screening score).
6. Has no more than moderate-severe (<=5) rating on PANSS positive symptom items P1 (delusions), P3 (hallucinatory behavior), P4 (excitement), P5 (grandiosity), P6 (suspiciousness), and/or G9 (unusual thought content), with a maximum of 2 of these items rated ‘5’; no more than moderate (<=4) rating on P2 (conceptual disorganization).
7. There is evidence that the participant has stable symptomatology >=3 months prior to the screening visit (example, no hospitalizations for schizophrenia, no emergency room admission due to symptoms of schizophrenia, no increase in level of psychiatric care due to worsening of symptoms of schizophrenia).
8. Have an adult informant who will be able to provide input for completing study rating scales, including the PANSS and SCORS (for example, a family member, social worker, caseworker, residential facility staff, or nurse who spends >=4 hours/week with the participant) and is considered reliable by the investigator. The informant must be able and willing to provide written informed consent and to participate in at least 1 in-person interview, then be able to provide continuing input by attending each clinical assessment visit or via participating in a telephone interview for other study visits that include the PANSS or SCORS endpoints.

Exclusion criteria for the study included:
1. Has a lifetime diagnosis of schizoaffective disorder; a lifetime diagnosis of bipolar disorder; or a lifetime diagnosis of obsessive compulsive disorder based on the MINI combined with the general psychiatric evaluation.
2. Has a recent (within the last 6 months) occurrence of panic disorder, depressive episode, or other comorbid psychiatric conditions currently requiring clinical attention based on the MINI for DSM-5 and the general psychiatric evaluation.
3. Has a diagnosis of substance use disorder (with the exception of nicotine dependence) within the preceding 6 months based on the MINI for DSM-5 and the general psychiatric evaluation.
4. Is participating in a formal structured nonpharmacological psychosocial therapeutic treatment program (cognitive remediation, cognitive-behavioral therapy, intensive symptom/vocational rehabilitation) for a duration of less than (<) 3 months before randomization. In addition, initiation of such nonpharmacological treatment programs is not permitted during study participation through the Day 84 visit.
5. The participant exhibits more than a minimal level of antipsychotic-induced parkinsonism symptoms, as documented by a score on the modified Simpson Angus Scale (SAS) (excluding item number 10, Akathisia) greater than (>) 6.
6. Has evidence of depression as measured by a Calgary Depression Scale Score (CDSS)>9.
7. Is considered by the investigator to be at imminent risk of suicide or injury to self, others, or property, or the participant has attempted suicide within the past year prior to screening. Participants who have positive answers on item number 4 or 5 on the Columbia-Suicide Severity Rating Scale (C-SSRS) (based on the past year) prior to randomization are excluded.
8. Has a history of brain trauma associated with loss of consciousness for >15 minutes.
9. Diagnosis of schizophrenia occurred prior to 12 years of age.
10. Has received electroconvulsive therapy within 6 months (180 days) before Screening.
11. Has a history of developmental intellectual disability or mental retardation.
12. Antipsychotic plasma levels for the participant's primary background antipsychotic are below the minimum acceptable concentration criteria per the Antipsychotic Reference document at the screening or placebo run-in visits. This criterion is not applicable to participants on a primary background antipsychotic for which a clinical assay is unavailable.
13. Is treatment resistant. Treatment resistance is defined as prior nonresponse of positive symptoms of schizophrenia to 2 courses of treatment with antipsychotics of different chemical classes for at least 4 weeks, each at doses considered to be effective.
14. Does not have a stable residence or is homeless.

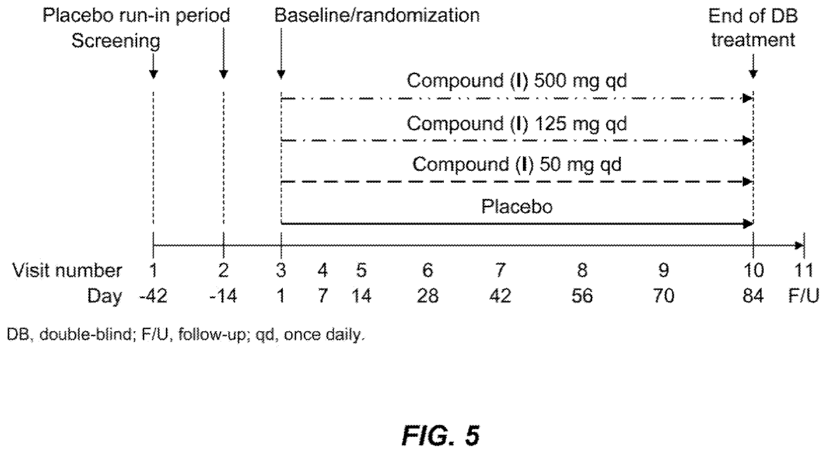

The study comprised a 28-day screening period, a 14-day single-blinded placebo run-in period to prospectively evaluate drug adherence and BNSS score stability, and a 12-week double-blinded treatment period (FIG. 5). The overall time to participate in the study was approximately 20 weeks. Participants who completed the study typically made 11 visits to the clinic, and were followed up for safety assessment 10 to 14 days after the last dose of Compound (I) (Day 98). Demographics, baseline characteristics, and efficacy assessments were based on the full analysis set, which included randomized participants who received at least one dose of the study drug during the treatment period (Table 6). The safety analysis set included randomized participants who received at least one dose of the study drug. All 256 enrolled patients were included in the full analysis set and safety analysis set.

TABLE 6
Baseline Demographic and Clinical Characteristics (Full Analysis Set)
for Patients Enrolled in the INTERACT Study

| | | Compound | Compound | Compound |
|---|---|---|---|---|
| | | (I) | (I) | (I) |
| | Placebo | 50 mg | 125 mg | 500 mg |
| | (n = 87) | (n = 58) | (n = 56) | (n = 55) |
| Age, years | | | | |
| Mean (SD) | 39.9 (11.11) | 39.9 (10.96) | 40.1 (9.80) | 40.1 (11.79) |
| Min., max. | 20, 60 | 21, 60 | 23, 60 | 18, 60 |
| Sex, n (%) | | | | |
| Male | 63 (72.4) | 38 (65.5) | 34 (60.7) | 33 (60.0) |
| Female | 24 (27.6) | 20 (34.5) | 22 (39.3) | 22 (40.0) |
| Race, n (%) | | | | |
| American Indian | 2 (2.3) | 0 | 1 (1.8) | 0 |
| or Alaska Native | | | | |
| Asian | 2 (2.3) | 0 | 3 (5.4) | 0 |
| Black or African | 10 (11.5) | 9 (15.5) | 9 (16.1) | 5 (9.1) |
| American | | | | |
| White | 70 (80.5) | 46 (79.3) | 42 (75.0) | 50 (90.9) |
| Not reported | 3 (3.4) | 3 (5.2) | 1 (1.8) | 0 |

PANSS NSFS at baseline

| | | | | |
|---|---|---|---|---|
| Mean (SD) | 24.6 (4.76) | 24.6 (4.25) | 24.1 (4.36) | 24.3 (5.02) |
| Min., max. | 14, 41 | 17, 35 | 16, 37 | 16, 37 |

BACS composite score at baseline

| | | | | |
|---|---|---|---|---|
| Mean (SD) | 33.4 (15.50) | 31.3 (15.61) | 32.6 (13.44) | 33.6 (12.67) |
| Min., max. | −11, 72 | −10, 63 | 0, 62 | 2, 62 |

SCoRS interviewer total score at baseline

| | | | | |
|---|---|---|---|---|
| Mean (SD) | 39.5 (9.90) | 39.6 (10.63) | 38.5 (10.21) | 41.3 (9.89) |
| Min., max. | 20, 61 | 21, 73 | 20, 62 | 23, 60 |

BACS, Brief Assessment of Cognition in Schizophrenia; max., maximum; Min., minimum; PANSS NSFS, Positive and Negative Syndrome Scale-Negative Symptom Factor Score; SCORS, Schizophrenia Cognition Rating Scale; SD, standard deviation.

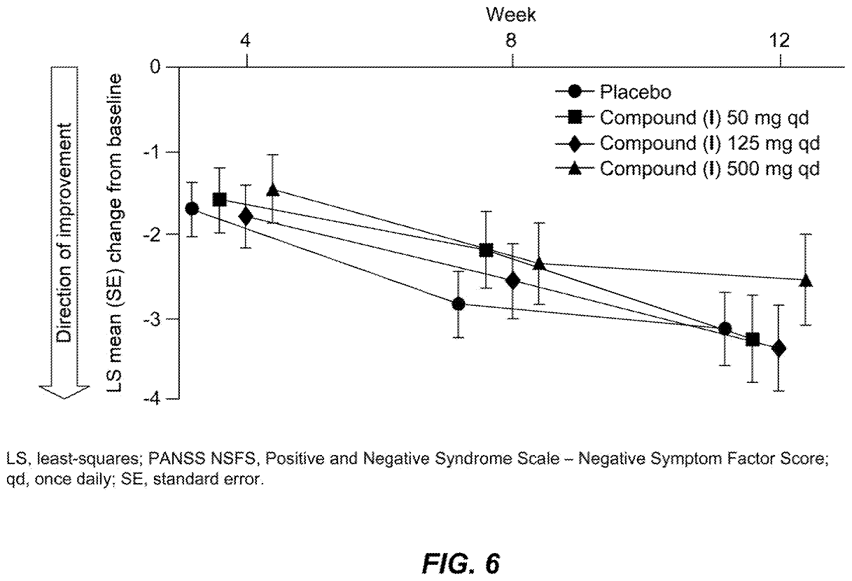

The primary endpoint evaluated was the change in negative symptoms, measured as the 12-week change from baseline in the Positive and Negative Syndrome Scale-Negative Symptom Factor Score (PANSS NSFS). For negative symptoms, no significant improvements in PANSS NSFS versus placebo were observed with Compound (1) 50 mg, 125 mg, or 500 mg at Week 12 (p=0.426, p=0.362 and p=0.808, respectively). From baseline to Week 12, least squares (LS) mean changes in PANSS NSFS were −3.3 (95% confidence interval [CI], −4.3 to −2.2), −3.4 (95% CI, −4.4 to −2.3) and −2.5 (95% CI, −3.6 to −1.5) with Compound (I) 50 mg, 125 mg, and 500 mg, respectively, and −3.1 (95% CI, −4.0 to −2.3) with placebo (FIG. 6). Additionally, no significant improvements were observed in negative symptoms as measured with the BNSS.

Safety endpoints included assessment of treatment-emergent adverse events (TEAEs). In total, 76 participants (29.7%) experienced a TEAE, of whom 23 (9%) were considered drug related by the investigator. Most TEAEs were mild or moderate in severity (Table 7). TEAEs occurring in more than 5 participants (greater than or equal to 2% of study participants) were headache, insomnia, and weight gain, which were observed at similar frequencies in the Compound (I) and placebo groups. Mild, moderate, and severe TEAEs occurred in 49 (19.1%), 24 (9.4%) and 3 (1.2%) participants, respectively. Severe events of rhabdomyolysis (placebo, n=1) and chronic cholecystitis (Compound (I) 50 mg, n=1) were not considered drug related. Severe schizophrenia was reported in one participant taking Compound (I) 125 mg and was considered a drug-related serious TEAE. Four serious TEAEs were reported in the placebo group; none were considered drug related. Five participants, including three taking placebo, experienced TEAEs resulting in discontinuation (blood creatine phosphokinase increased, liver function test increased, psychiatric disorders). No deaths were reported, and no clinically significant findings in safety, laboratory, vital signs, or electrocardiogram assessments were observed across groups.

TABLE 7
Incidence of TEAEs (Safety Analysis Set) for Patients Enrolled in the
INTERACT Study

| | | Compound | Compound | Compound |
|---|---|---|---|---|
| | | (I) | (I) | (I) |
| | Placebo | 50 mg | 125 mg | 500 mg |
| | (N = 87) | (N = 58) | (N = 56) | (N = 55) |
| Patients with any TEAEs | 30 (34.5) | 13 (22.4) | 17 (30.4) | 16 (29.1) |
| Mild | 18 (20.7) | 10 (17.2) | 13 (23.2) | 8 (14.5) |
| Moderate | 11 (12.6) | 2 (3.4) | 3 (5.4) | 8 (14.5) |
| Severe | 1 (1.1) | 1 (1.7) | 1 (1.8) | 0 |
| TEAEs related to study | 12 (13.8) | 0 | 4 (7.1) | 7 (12.7) |
| drug^{a} | | | | |
| Serious adverse events | 4 (4.6) | 0 | 1 (1.8) | 0 |
| Deaths | 0 | 0 | 0 | 0 |
| TEAEs leading to study | 3 (3.4) | 0 | 1 (1.8) | 1 (1.8) |
| discontinuation | | | | |
| Frequent TEAEs | | | | |
| Headache | 4 (4.6) | 0 | 5 (8.9) | 2 (3.6) |
| Insomnia | 3 (3.4) | 0 | 2 (3.6) | 2 (3.6) |
| Increased weight | 3 (3.4) | 2 (3.4) | 0 | 0 |

To assess changes in cognitive impairment associated with schizophrenia (CIAS), secondary endpoints included the 12-week change from baseline in the Brief Assessment of Cognition in Schizophrenia (BACS) composite score, a measure of cognition across multiple domains, and the Schizophrenia Cognition Rating Scale (SCORS) score, a measure of day-to-day cognitive functioning requiring input from an adult informant. Secondary endpoints were not corrected for multiplicity.

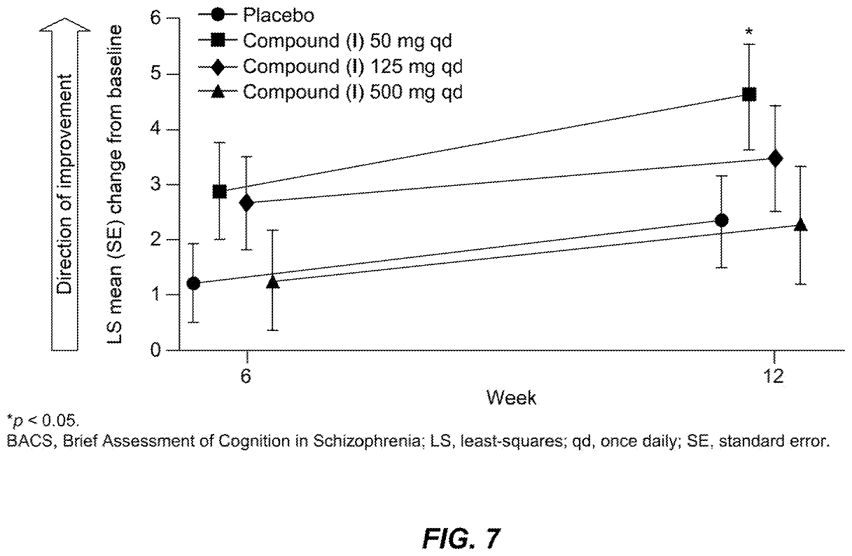

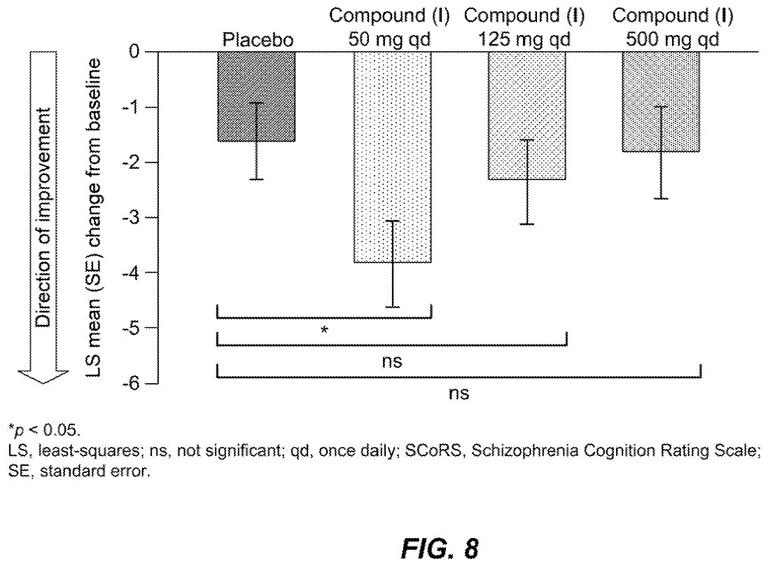

For CIAS, nominally significant improvements in cognitive assessment and patient cognitive functioning were observed with Compound (I) 50 mg versus placebo in the BACS composite score (p=0.031; effect size, 0.4, not corrected for multiplicity, FIG. 7) and the SCORS interviewer total score (p=0.011; effect size, 0.4), not corrected for multiplicity, FIG. 8, but not with Compound (I) 125 mg or 500 mg. For the BACS composite score, a 12-week LS mean change from baseline of 4.6 (95% CI, 2.7 to 6.5) was observed with Compound (I) 50 mg, versus 2.3 (95% CI, 0.7 to 3.9) with placebo. For the SCORS interviewer total score, a 12-week LS mean change from baseline of −3.8 (95% CI, −5.3 to −2.3) was observed with Compound (I) 50 mg versus −1.6 (95% CI, −2.9 to −0.3) with placebo.

The BACS is specifically designed to measure treatment-related improvements in cognition and includes alternate forms. BACS is a reliable and sensitive measure of cognitive function in schizophrenia. The BACS is a cognition assessment battery that assesses 6 domains of cognitive function found to be consistently impaired in schizophrenia: verbal memory; working memory; motor speed; attention; executive functions; and verbal fluency. The primary measure from each test of the BACS is standardized by creating T-scores whereby the mean of the test session of a matched reference healthy participant is set to 50 and the standard deviation set to 10. Preliminary analyses of results from the BACS assessment are summarized in Tables 10-16.

The SCORS is an interview-based measure of cognitive functioning that was developed to specifically assess aspects of cognitive functioning in participants with schizophrenia. The items assess the 7 cognitive domains of attention, memory, reasoning and problem solving, working memory, processing speed, language functions, and social cognition. The SCORS global total scores is the sum of the 20 items, and it varies from 20 to 80 with 20 being the best outcome and 80 being the worst. Preliminary analysis of results from the SCORS assessment is summarized in Table 17.

Compound (I) did not show significant efficacy in the treatment of negative symptoms of schizophrenia at the three doses evaluated in the INTERACT study. However, Compound (I) 50 mg showed an improvement in cognitive test performance and also in self-rated and informant-rated day-to-day cognitive functioning, as measured by the BACS composite score and the SCORS interviewer total score, respectively, with a clinically meaningful effect size of 0.4 for each. For CIAS, an inverted U-shaped dose response was observed with Compound (I) 50 mg, 125 mg, and 500 mg. As described in Example 1, inverted U-shaped dose responses were also observed in long-term potentiation studies in in vitro models that were conducted in parallel with the INTERACT study. Additional clinical research is warranted to further evaluate this efficacy signal. Consistent with prior clinical experience, Compound (I) was generally well tolerated in the INTERACT study.

TABLE 8
Disposition of Subjects (Preliminary Analysis)

| | Number of Subjects (%) | | | | | | | | | | | |
|---|---|---|---|---|---|---|---|---|---|---|---|---|
| | | | Compound | | Compound | | Compound | | Compound | | | |
| | | | (I) | | (I) | | (I) | | (I) | | | |
| | Placebo | | 50 mg | | 125 mg | | 500 mg | | Overall | | Overall | |
| | (N = 87) | | (N = 58) | | (N = 56) | | (N = 55) | | (N = 169) | | (N = 256) | |
| Received At Least | 87 | | 58 | | 56 | | 55 | | 169 | | 256 | |
| One Dose of Study | | | | | | | | | | | | |
| Drug | | | | | | | | | | | | |
| Completed the Study | 76 | (87.4) | 53 | (91.4) | 52 | (92.9) | 47 | (85.5) | 152 | (89.9) | 228 | (89.1) |
| Prematurely | | | | | | | | | | | | |
| Discontinued Study | 11 | (12.6) | 5 | (8.6) | 4 | (7.1) | 8 | (14.5) | 17 | (10.1) | 28 | (10.9) |
| Drug | | | | | | | | | | | | |
| Prematurely | 11 | (12.6) | 5 | (8.6) | 4 | (7.1) | 8 | (14.5) | 17 | (10.1) | 28 | (10.9) |
| Discontinued Study | | | | | | | | | | | | |
| Reason for | | | | | | | | | | | | |
| Discontinuation of | | | | | | | | | | | | |
| Study Drug | | | | | | | | | | | | |
| Adverse Event | 3 | (27.3) | 0 | | 1 | (25.0) | 1 | (12.5) | 2 | (11.8) | 5 | (17.9) |
| Protocol | 0 | | 0 | | 0 | | 0 | | 0 | | 0 | |
| Deviation | | | | | | | | | | | | |
| Lost to Follow-Up | 1 | (9.1) | 0 | | 0 | | 0 | | 0 | | 1 | (3.6) |
| Withdrawal by | 4 | (36.4) | 3 | (60.0) | 2 | (50.0) | 4 | (50.0) | 9 | (52.9) | 13 | (46.4) |
| Subject | | | | | | | | | | | | |
| Study Terminated | 0 | | 0 | | 0 | | 0 | | 0 | | 0 | |
| by Sponsor | | | | | | | | | | | | |
| Pregnancy | 0 | | 0 | | 0 | | 0 | | 0 | | 0 | |
| Unsatisfactory | 1 | (9.1) | 0 | | 0 | | 0 | | 0 | | 1 | (3.6) |
| Therapeutic Response | | | | | | | | | | | | |
| Noncompliance | | | | | | | | | | | | |
| with Study Drug | | | | | | | | | | | | |
| During the | 1 | (9.1) | 2 | (40.0) | 1 | (25.0) | 1 | (12.5) | 4 | (23.5) | 5 | (17.9) |
| Double-Blind | | | | | | | | | | | | |
| Treatment Period | | | | | | | | | | | | |
| Significant Risk | 0 | | 0 | | 0 | | 0 | | 0 | | 0 | |
| of Suicide | | | | | | | | | | | | |
| Symptomatic | 0 | | 0 | | 0 | | 0 | | 0 | | 0 | |
| Deterioration | | | | | | | | | | | | |
| Other | 1 | (9.1) | 0 | | 0 | | 2 | (25.0) | 2 | (11.8) | 3 | (10.7) |
| Reason for | | | | | | | | | | | | |
| Discontinuation of | | | | | | | | | | | | |
| Study | | | | | | | | | | | | |
| Death | 0 | | 0 | | 0 | | 0 | | 0 | | 0 | |
| Adverse Event | 2 | (18.2) | 0 | | 1 | (25.0) | 1 | (12.5) | 2 | (11.8) | 4 | (14.3) |
| Protocol | 0 | | 0 | | 0 | | 0 | | 0 | | 0 | |
| Deviation | | | | | | | | | | | | |
| Lost to Follow-Up | 1 | (9.1) | 0 | | 0 | | 0 | | 0 | | 1 | (3.6) |
| Withdrawal by | 4 | (36.4) | 3 | (60.0) | 2 | (50.0) | 3 | (37.5) | 8 | (47.1) | 12 | (42.9) |
| Subject | | | | | | | | | | | | |
| Study Terminated | 0 | | 0 | | 0 | | 0 | | 0 | | 0 | |
| by Sponsor | | | | | | | | | | | | |
| Pregnancy | 0 | | 0 | | 0 | | 0 | | 0 | | 0 | |
| Unsatisfactory | 1 | (9.1) | 0 | | 0 | | 0 | | 0 | | 1 | (3.6) |
| Therapeutic Response | | | | | | | | | | | | |
| Noncompliance | | | | | | | | | | | | |
| with Study Drug | | | | | | | | | | | | |
| During the | 1 | (9.1) | 2 | (40.0) | 1 | (25.0) | 1 | (12.5) | 4 | (23.5) | 5 | (17.9) |

| Double-Blind | 0 | | 0 | 0 | 0 | | 0 | | 0 | |
|---|---|---|---|---|---|---|---|---|---|---|
| Treatment Period | | | | | | | | | | |
| Significant Risk | 0 | | 0 | 0 | 0 | | 0 | | 0 | |
| of Suicide | | | | | | | | | | |
| Symptomatic | 0 | | 0 | 0 | 0 | | 0 | | 0 | |
| Deterioration | | | | | | | | | | |
| Other | 2 | (18.2) | 0 | 0 | 3 | (37.5) | 3 | (17.6) | 5 | (17.9) |

Note 1:
Percentages are based on the total number of subjects in the analysis set for each treatment group that received at least one dose of study drug.
Note 2:
Percentages for reason for discontinuation of study drug/study are based on the total number of subjects who prematurely discontinued study drug/study.

TABLE 9
Demographic and Baseline Characteristics (Preliminary Analysis)

| | | | Compound (I) | | Compound (I) | | Compound (I) | | Compound (I) | | | |
|---|---|---|---|---|---|---|---|---|---|---|---|---|
| | Placebo | | 50 mg | | 125 mg | | 500 mg | | Overall | | Overall | |
| | (N = 87) | | (N = 58) | | (N = 56) | | (N = 55) | | (N = 169) | | (N = 256) | |
| Age (Years) | | | | | | | | | | | | |
| N | 87 | | 58 | | 56 | | 55 | | 169 | | 256 | |
| Mean (SD) | 39.9 | (11.11) | 39.9 | (10.96) | 40.1 | (9.80) | 40.1 | (11.79) | 40.0 | (10.81) | 40.0 | (10.89) |
| Median | 38.0 | | 39.0 | | 40.5 | | 39.0 | | 39.0 | | 39.0 | |
| Minimum, | 20, 60 | | 21, 60 | | 23, 60 | | 18, 60 | | 18, 60 | | 18, 60 | |
| Maximum | | | | | | | | | | | | |
| Age Categories | | | | | | | | | | | | |
| (n (%)) | | | | | | | | | | | | |
| Age <35 years | 30 | (34.5) | 19 | (32.8) | 18 | (32.1) | 18 | (32.7) | 55 | (32.5) | 85 | (33.2) |
| Age >=35 years | 57 | (65.5) | 39 | (67.2) | 38 | (67.9) | 37 | (67.3) | 114 | (67.5) | 171 | (66.8) |
| Ethnicity (n (%)) | | | | | | | | | | | | |
| Hispanic or | 3 | (3.4) | 2 | (3.4) | 2 | (3.6) | 1 | (1.8) | 5 | (3.0) | 8 | (3.1) |
| Latino | | | | | | | | | | | | |
| Not-Hispanic | 19 | (21.8) | 12 | (20.7) | 18 | (32.1) | 8 | (14.5) | 38 | (22.5) | 57 | (22.3) |
| and Latino | | | | | | | | | | | | |
| Not Reported | 65 | (74.7) | 44 | (75.9) | 36 | (64.3) | 46 | (83.6) | 126 | (74.6) | 191 | (74.6) |
| Unknown | 0 | | 0 | | 0 | | 0 | | 0 | | 0 | |
| Gender (n (%)) | | | | | | | | | | | | |
| Male | 63 | (72.4) | 38 | (65.5) | 34 | (60.7) | 33 | (60.0) | 105 | (62.1) | 168 | (65.6) |
| Female | 24 | (27.6) | 20 | (34.5) | 22 | (39.3) | 22 | (40.0) | 64 | (37.9) | 88 | (34.4) |
| Race (n (%)) | | | | | | | | | | | | |
| American Indian | 2 | (2.3) | 0 | | 1 | (1.8) | 0 | | 1 | (0.6) | 3 | (1.2) |
| or Alaska Native | | | | | | | | | | | | |
| Asian | 2 | (2.3) | 0 | | 3 | (5.4) | 0 | | 3 | (1.8) | 5 | (2.0) |
| Black or African | 10 | (11.5) | 9 | (15.5) | 9 | (16.1) | 5 | (9.1) | 23 | (13.6) | 33 | (12.9) |
| American | | | | | | | | | | | | |
| Native Hawaiian | 0 | | 0 | | 0 | | 0 | | 0 | | 0 | |
| or Other Pacific | | | | | | | | | | | | |
| Islander | | | | | | | | | | | | |
| White | 70 | (80.5) | 46 | (79.3) | 42 | (75.0) | 50 | (90.9) | 138 | (81.7) | 208 | (81.3) |
| Multiracial | 0 | | 0 | | 0 | | 0 | | 0 | | 0 | |
| Not Reported | 3 | (3.4) | 3 | (5.2) | 1 | (1.8) | 0 | | 4 | (2.4) | 7 | (2.7) |
| Country (n (%)) | | | | | | | | | | | | |
| Bulgaria | 18 | (20.7) | 12 | (20.7) | 13 | (23.2) | 11 | (20.0) | 36 | (21.3) | 54 | (21.1) |
| Czech Republic | 5 | (5.7) | 4 | (6.9) | 2 | (3.6) | 3 | (5.5) | 9 | (5.3) | 14 | (5.5) |
| Italy | 10 | (11.5) | 5 | (8.6) | 2 | (3.6) | 6 | (10.9) | 13 | (7.7) | 23 | (9.0) |
| Poland | 3 | (3.4) | 0 | | 2 | (3.6) | 1 | (1.8) | 3 | (1.8) | 6 | (2.3) |
| Russian | 7 | (8.0) | 6 | (10.3) | 4 | (7.1) | 5 | (9.1) | 15 | (8.9) | 22 | (8.6) |
| Federation | | | | | | | | | | | | |
| Serbia | 5 | (5.7) | 1 | (1.7) | 3 | (5.4) | 3 | (5.5) | 7 | (4.1) | 12 | (4.7) |
| Spain | 3 | (3.4) | 2 | (3.4) | 1 | (1.8) | 3 | (5.5) | 6 | (3.6) | 9 | (3.5) |
| Ukraine | 14 | (16.1) | 14 | (24.1) | 9 | (16.1) | 14 | (25.5) | 37 | (21.9) | 51 | (19.9) |
| United States | 22 | (25.3) | 14 | (24.1) | 20 | (35.7) | 9 | (16.4) | 43 | (25.4) | 65 | (25.4) |
| Height (cm) | | | | | | | | | | | | |
| N | 87 | | 58 | | 56 | | 55 | | 169 | | 256 | |
| Mean (SD) | 173.65 | (8.568) | 175.11 | (8.992) | 172.23 | (9.884) | 171.14 | (8.951) | 172.86 | (9.382) | 173.13 | (9.104) |
| Median | 174.00 | | 175.60 | | 173.00 | | 172.00 | | 174.00 | | 174.00 | |
| Minimum, | 148.0, 193.0 | | 154.0, 197.0 | | 146.0, 193.0 | | 150.0, 188.5 | | 146.0, 197.0 | | 146.0, 197.0 | |
| Maximum | | | | | | | | | | | | |
| Weight at | | | | | | | | | | | | |
| Screening (kg) | | | | | | | | | | | | |
| N | 87 | | 58 | | 56 | | 55 | | 169 | | 256 | |
| Mean (SD) | 86.80 | (16.474) | 84.86 | (18.961) | 83.70 | (16.816) | 81.96 | (16.346) | 83.53 | (17.374) | 84.64 | (17.112) |
| Median | 85.00 | | 81.30 | | 85.50 | | 82.30 | | 83.00 | | 84.15 | |
| Minimum, | 51.6, 135.6 | | 52.0, 140.9 | | 46.0, 132.9 | | 47.8, 115.0 | | 46.0, 140.9 | | 46.0, 140.9 | |
| Maximum | | | | | | | | | | | | |
| BMI (kg/m\hat{ }2) | | | | | | | | | | | | |
| N | 87 | | 58 | | 56 | | 55 | | 169 | | 256 | |
| Mean (SD) | 28.70 | (4.497) | 27.55 | (5.061) | 28.18 | (5.081) | 27.95 | (5.081) | 27.89 | (5.051) | 28.16 | (4.876) |

| | | | | | | |
|---|---|---|---|---|---|---|
| Median | 28.70 | 27.55 | 28.05 | 27.40 | 27.70 | 28.00 |
| Minimum, | 18.1, 40.2 | 19.2, 37.0 | 19.3, 39.7 | 18.5, 39.6 | 18.5, 39.7 | 18.1, 40.2 |
| Maximum | | | | | | |

Note 1:
Percentages are based on the total number of subjects in the analysis set for each treatment group.
Note 2:
If a subject documented more than one race category on the CRF, the subject is only included under the Multiracial category.
Note 3:
Age at Screening is calculated from the informed consent date.
Note 4:
Hispanic ethnicity is entered only for U.S. sites. Ethnicity for non-U.S. sites will be categorized as Not Reported.

TABLE 10
Analysis of Change from Baseline on the BACS Composite Score by Visit
(Preliminary Analysis)

| | | | Compound | Compound |
|---|---|---|---|---|
| | | Compound (I) | (I) | (I) |
| Visit | Placebo | 50 mg | 125 mg | 500 mg |
| Statistic | (N = 87) | (N = 58) | (N = 56) | (N = 55) |
| Baseline (a) | | | | |
| N | 87 | 58 | 56 | 55 |
| Mean (SD) | 34.9 (14.84) | 33.1 (14.44) | 33.7 (13.65) | 34.1 (12.80) |
| Median | 35.0 | 32.5 | 34.0 | 35.0 |
| Minimum, Maximum | −7, 72 | −1, 63 | 0, 62 | 2, 62 |
| Week 6 | | | | |
| N | 79 | 56 | 56 | 51 |
| Mean (SD) | 34.9 (15.67) | 35.4 (14.53) | 35.6 (14.55) | 35.6 (13.84) |
| Median | 36.0 | 34.0 | 37.0 | 38.0 |
| Minimum, Maximum | −16, 70 | −1, 73 | 2, 72 | −7, 63 |
| Change from Baseline to Week 6 (a) | | | | |
| N | 79 | 56 | 56 | 51 |
| Mean (SD) | 0.4 (5.28) | 1.7 (5.81) | 1.9 (4.95) | 1.5 (7.57) |
| Median | 1.0 | 2.0 | 2.0 | 1.0 |
| Minimum, Maximum | −13, 18 | −12, 19 | −7, 13 | −24, 20 |
| Adjusted Change from Baseline | | | | |
| to Week 6 | | | | |
| LS Mean (SE) | −0.4 (0.70) | 0.9 (0.81) | 1.4 (0.80) | 0.5 (0.87) |
| 95% CI | (−1.8, 1.0) | (−0.7, 2.5) | (−0.2, 3.0) | (−1.2, 2.2) |
| Difference from Placebo for Week | | | | |
| 6 | | | | |
| LS Mean (SE) | | 1.3 (1.01) | 1.8 (1.02) | 0.9 (1.04) |
| 95% CI | | (−0.7, 3.3) | (−0.2, 3.8) | (−1.1, 3.0) |
| p-value (unadjusted) | | 0.096 | 0.038 | 0.194 |
| Week 12 | | | | |
| N | 73 | 52 | 50 | 45 |
| Mean (SD) | 36.4 (15.31) | 37.7 (13.30) | 37.1 (15.02) | 37.6 (13.86) |
| Median | 36.0 | 37.5 | 38.5 | 40.0 |
| Minimum, Maximum | −3, 74 | −8, 67 | −8, 71 | 5, 65 |
| Change from Baseline to Week 12 | | | | |
| (a) | | | | |
| N | 73 | 52 | 50 | 45 |
| Mean (SD) | 1.4 (5.47) | 3.8 (5.91) | 2.9 (6.54) | 3.0 (7.98) |
| Median | 1.0 | 3.0 | 3.5 | 3.0 |
| Minimum, Maximum | −10, 21 | −10, 19 | −11, 16 | −14, 26 |
| Adjusted Change from Baseline to | | | | |
| Week 12 | | | | |
| LS Mean (SE) | 0.7 (0.78) | 2.7 (0.92) | 2.3 (0.91) | 1.5 (0.99) |
| 95% CI | (−0.8, 2.3) | (0.9, 4.5) | (0.5, 4.1) | (−0.5, 3.5) |
| Difference from Placebo for Week | | | | |
| 12 | | | | |
| LS Mean (SE) | | 1.9 (1.15) | 1.5 (1.16) | 0.8 (1.20) |
| 95% CI | | (−0.3, 4.2) | (−0.8, 3.8) | (−1.6, 3.1) |
| p-value (unadjusted) | | 0.046 | 0.095 | 0.260 |

CI—Confidence interval;
LS—Least squares;
SE—Standard error.
Note 1:
The change from Baseline on the BACS composite score is analyzed using the mixed model for repeated measures (MMRM) with Baseline BACS composite score as a covariate; pooled site, visit, treatment, and categorical age (randomization factor) as fixed factors; and treatment-by-visit and Baseline-by-visit interactions. Based on a Missing at Random Assumption, this analysis will be performed using observed case only. P-values are one-sided, with the alternative hypothesis that Compound (I) is superior to placebo in the clinically favorable direction.
Note 2:
(a) Baseline is defined as the last observed value before the first dose of study medication.

TABLE 11
Analysis of Change from Baseline on the BACS Verbal Memory Cognition
Domain by Visit (Preliminary Analysis)

| | | | Compound | Compound |
|---|---|---|---|---|
| | | Compound (I) | (I) | (I) |
| Visit | Placebo | 50 mg | 125 mg | 500 mg |
| Statistic | (N = 87) | (N = 58) | (N = 56) | (N = 55) |
| Baseline (a) | | | | |
| N | 87 | 58 | 56 | 55 |
| Mean (SD) | 43.1 (11.60) | 39.6 (11.65) | 40.6 (12.82) | 40.5 (12.53) |
| Median | 44.0 | 40.5 | 40.0 | 41.0 |
| Minimum, Maximum | 19, 73 | 12, 64 | 5, 70 | 5, 72 |
| Week 6 | | | | |
| N | 79 | 56 | 56 | 51 |
| Mean (SD) | 41.8 (13.69) | 41.7 (11.59) | 42.4 (12.62) | 42.1 (12.06) |
| Median | 42.0 | 44.5 | 42.0 | 42.0 |
| Minimum, Maximum | 3, 69 | 9, 68 | 16, 75 | 10, 72 |
| Change from Baseline to Week | | | | |
| 6 (a) | | | | |
| N | 79 | 56 | 56 | 51 |
| Mean (SD) | −0.8 (7.10) | 1.6 (7.40) | 1.8 (7.71) | 1.8 (8.65) |
| Median | 0.0 | 0.0 | 2.5 | 2.0 |
| Minimum, Maximum | −17, 16 | −12, 21 | −17, 19 | −15, 22 |
| Adjusted Change from Baseline | | | | |
| to Week 6 | | | | |
| LS Mean (SE) | −1.3 (0.88) | 0.6 (1.04) | 1.2 (1.01) | 0.7 (1.11) |
| 95% CI | (−3.1, 0.4) | (−1.5, 2.6) | (−0.8, 3.2) | (−1.5, 2.8) |
| Difference from Placebo for Week | | | | |
| 6 | | | | |
| LS Mean (SE) | | 1.9 (1.29) | 2.5 (1.29) | 2.0 (1.33) |
| 95% CI | | (−0.6, 4.4) | (0.0, 5.1) | (−0.6, 4.6) |
| p-value (unadjusted) | | 0.071 | 0.025 | 0.070 |
| Week 12 | | | | |
| N | 72 | 52 | 50 | 45 |
| Mean (SD) | 42.7 (11.04) | 42.9 (12.41) | 42.3 (13.54) | 41.8 (13.82) |
| Median | 42.0 | 43.5 | 42.5 | 41.0 |
| Minimum, Maximum | 19, 69 | 14, 70 | −1, 72 | 5, 76 |
| Change from Baseline to Week | | | | |
| 12 (a) | | | | |
| N | 72 | 52 | 50 | 45 |
| Mean (SD) | −0.1 (7.26) | 3.2 (7.87) | 1.6 (8.07) | 1.6 (10.04) |
| Median | −0.5 | 4.0 | 1.0 | 0.0 |
| Minimum, Maximum | −17, 23 | −11, 26 | −17, 23 | −20, 33 |
| Adjusted Change from Baseline to | | | | |
| Week 12 | | | | |
| LS Mean (SE) | −0.7 (0.98) | 1.9 (1.13) | 0.8 (1.13) | 0.0 (1.23) |
| 95% CI | (−2.6, 1.3) | (−0.3, 4.1) | (−1.5, 3.0) | (−2.4, 2.4) |
| Difference from Placebo for Week | | | | |
| 12 | | | | |
| LS Mean (SE) | | 2.6 (1.43) | 1.4 (1.44) | 0.7 (1.49) |
| 95% CI | | (−0.3, 5.4) | (−1.4, 4.3) | (−2.3, 3.6) |
| p-value (unadjusted) | | 0.038 | 0.164 | 0.329 |

CI—Confidence interval;
LS—Least squares;
SE—Standard error.
Note 1:
The change from Baseline on the f Verbal Memory Cognition Domain are analyzed using the mixed model for repeated measures (MMRM) with Baseline BACS Verbal Memory Cognition Domain as a covariate; pooled site, visit, treatment, and categorical age (randomization factor) as fixed factors; and treatment-by-visit and Baseline-by-visit interactions. Based on a Missing at Random Assumption, this analysis will be performed using observed case only. P-values are one-sided, with the alternative hypothesis that Compound (I) is superior to placebo in the clinically favorable direction.
Note 2:
(a) Baseline is defined as the last observed value before the first dose of study medication.

TABLE 12
Analysis of Change from Baseline on the BACS Working Memory Cognition
Domain by Visit (Preliminary Analysis)

| | | | Compound | Compound |
|---|---|---|---|---|
| | | Compound (I) | (I) | (I) |
| Visit | Placebo | 50 mg | 125 mg | 500 mg |
| Statistic | (N = 87) | (N = 58) | (N = 56) | (N = 55) |
| Baseline (a) | | | | |
| N | 87 | 58 | 56 | 55 |
| Mean (SD) | 39.2 (12.18) | 39.3 (14.00) | 39.5 (11.17) | 39.6 (12.95) |
| Median | 38.0 | 37.5 | 40.0 | 41.0 |
| Minimum, Maximum | 14, 67 | 13, 66 | 15, 59 | 7, 64 |
| Week 6 | | | | |
| N | 79 | 56 | 56 | 51 |
| Mean (SD) | 39.8 (12.94) | 40.8 (14.10) | 41.0 (11.17) | 40.1 (13.75) |
| Median | 39.0 | 42.0 | 41.0 | 43.0 |
| Minimum, Maximum | 4, 66 | 14, 72 | 18, 66 | 5, 60 |
| Change from Baseline to Week | | | | |
| 6 (a) | | | | |
| N | 79 | 56 | 56 | 51 |
| Mean (SD) | 1.2 (6.84) | 1.0 (6.60) | 1.5 (5.89) | 0.5 (7.99) |
| Median | 2.0 | 0.0 | 2.0 | 0.0 |
| Minimum, Maximum | −20, 28 | −12, 15 | −12, 14 | −18, 18 |
| Adjusted Change from Baseline | | | | |
| to Week 6 | | | | |
| LS Mean (SE) | 0.0 (0.79) | 0.0 (0.92) | 0.9 (0.90) | −0.9 (0.98) |
| 95% CI | (−1.5, 1.6) | (−1.8, 1.8) | (−0.9, 2.6) | (−2.8, 1.0) |
| Difference from Placebo for Week | | | | |
| 6 | | | | |
| LS Mean (SE) | | 0.0 (1.15) | 0.8 (1.16) | −0.9 (1.19) |
| 95% CI | | (−2.3, 2.2) | (−1.5, 3.1) | (−3.3, 1.4) |
| p-value (unadjusted) | | 0.507 | 0.239 | 0.788 |
| Week 12 | | | | |
| N | 73 | 52 | 50 | 45 |
| Mean (SD) | 40.5 (13.94) | 42.0 (13.95) | 41.7 (13.58) | 42.6 (13.20) |
| Median | 38.0 | 42.5 | 40.0 | 45.0 |
| Minimum, Maximum | 12, 75 | 10, 63 | 10, 67 | 14, 67 |
| Change from Baseline to Week 12 | | | | |
| (a) | | | | |
| N | 73 | 52 | 50 | 45 |
| Mean (SD) | 2.0 (8.69) | 2.1 (6.61) | 1.8 (7.40) | 1.7 (7.39) |
| Median | 3.0 | 1.0 | 0.0 | 0.0 |
| Minimum, Maximum | −31, 23 | −11, 17 | −16, 20 | −10, 20 |
| Adjusted Change from Baseline to | | | | |
| Week 12 | | | | |
| LS Mean (SE) | 0.7 (0.91) | 0.9 (1.06) | 1.0 (1.06) | 0.2 (1.15) |
| 95% CI | (−1.1, 2.4) | (−1.1, 3.0) | (−1.1, 3.1) | (−2.1, 2.4) |
| Difference from Placebo for Week | | | | |
| 12 | | | | |
| LS Mean (SE) | | 0.3 (1.34) | 0.3 (1.35) | −0.5 (1.40) |
| 95% CI | | (−2.3, 2.9) | (−2.3, 3.0) | (−3.2, 2.3) |
| p-value (unadjusted) | | 0.413 | 0.406 | 0.637 |

CI—Confidence interval;
LS—Least squares;
SE—Standard error.
Note 1:
The change from Baseline on the BACS Working Memory Cognition Domain are analyzed using the mixed model for repeated measures (MMRM) with Baseline BACS Working Memory Cognition Domain as a covariate; pooled site, visit, treatment, and categorical age (randomization factor) as fixed factors; and treatment-by-visit and Baseline-by-visit interactions. Based on a Missing at Random Assumption, this analysis will be performed using observed case only. P-values are one-sided, with the alternative hypothesis that Compound (I) is superior to placebo in the clinically favorable direction.
Note 2:
(a) Baseline is defined as the last observed value before the first dose of study medication.

TABLE 13
Analysis of Change from Baseline on the BACS Motor Speed Cognition Domain
by Visit (Preliminary Analysis)

| | | | Compound | Compound |
|---|---|---|---|---|
| | | Compound (I) | (I) | (I) |
| Visit | Placebo | 50 mg | 125 mg | 500 mg |
| Statistic | (N = 87) | (N = 58) | (N = 56) | (N = 55) |
| Baseline (a) | | | | |
| N | 86 | 58 | 56 | 55 |
| Mean (SD) | 41.7 (14.10) | 39.6 (14.11) | 39.6 (9.64) | 40.5 (12.13) |
| Median | 40.5 | 37.0 | 39.5 | 39.0 |
| Minimum, Maximum | −4, 73 | 13, 78 | 20, 60 | 3, 73 |
| Week 6 | | | | |
| N | 79 | 56 | 56 | 51 |
| Mean (SD) | 43.1 (14.70) | 40.6 (13.61) | 39.6 (10.80) | 40.7 (12.38) |
| Median | 43.0 | 40.0 | 39.0 | 41.0 |
| Minimum, Maximum | 4, 73 | 18, 77 | 19, 68 | 1, 66 |
| Change from Baseline to Week | | | | |
| 6 (a) | | | | |
| N | 78 | 56 | 56 | 51 |
| Mean (SD) | 0.9 (8.44) | 1.7 (5.73) | 0.0 (5.97) | 0.5 (9.53) |
| Median | 0.0 | 1.0 | 0.5 | 2.0 |
| Minimum, Maximum | −29, 33 | −10, 15 | −10, 21 | −33, 25 |
| Adjusted Change from Baseline | | | | |
| to Week 6 | | | | |
| LS Mean (SE) | −0.1 (0.86) | 0.1 (1.00) | −1.0 (0.98) | −1.3 (1.07) |
| 95% CI | (−1.8, 1.6) | (−1.9, 2.1) | (−2.9, 1.0) | (−3.4, 0.9) |
| Difference from Placebo for Week | | | | |
| 6 | | | | |
| LS Mean (SE) | | 0.2 (1.26) | −0.9 (1.26) | −1.1 (1.30) |
| 95% CI | | (−2.3, 2.7) | (−3.4, 1.6) | (−3.7, 1.4) |
| p-value (unadjusted) | | 0.440 | 0.753 | 0.812 |
| Week 12 | | | | |
| N | 72 | 52 | 50 | 45 |
| Mean (SD) | 42.9 (14.36) | 42.0 (12.93) | 42.6 (10.35) | 41.3 (11.31) |
| Median | 41.5 | 41.5 | 42.0 | 40.0 |
| Minimum, Maximum | 8, 71 | 9, 74 | 19, 70 | 1, 63 |
| Change from Baseline to Week | | | | |
| 12 (a) | | | | |
| N | 71 | 52 | 50 | 45 |
| Mean (SD) | 0.1 (6.74) | 2.6 (6.80) | 2.5 (7.60) | 2.0 (6.58) |
| Median | 0.0 | 1.0 | 2.0 | 2.0 |
| Minimum, Maximum | −16, 14 | −7, 23 | −13, 24 | −14, 17 |
| Adjusted Change from Baseline to | | | | |
| Week 12 | | | | |
| LS Mean (SE) | −0.5 (0.81) | 1.0 (0.93) | 1.3 (0.93) | −0.2 (1.02) |
| 95% CI | (−2.1, 1.1) | (−0.9, 2.8) | (−0.6, 3.1) | (−2.2, 1.8) |
| Difference from Placebo for Week | | | | |
| 12 | | | | |
| LS Mean (SE) | | 1.5 (1.17) | 1.8 (1.18) | 0.3 (1.22) |
| 95% CI | | (−0.8, 3.8) | (−0.5, 4.1) | (−2.1, 2.7) |
| p-value (unadjusted) | | 0.101 | 0.067 | 0.405 |

CI—Confidence interval;
LS—Least squares;
SE—Standard error.
Note 1:
The change from Baseline on the BACS Motor Speed Cognition Domain are analyzed using the mixed model for repeated measures (MMRM) with Baseline BACS Motor Speed Cognition Domain as a covariate; pooled site, visit, treatment, and categorical age (randomization factor) as fixed factors; and treatment-by-visit and Baseline-by-visit interactions. Based on a Missing at Random Assumption, this analysis will be performed using observed case only. P-values are one-sided, with the alternative hypothesis that Compound (I) is superior to placebo in the clinically favorable direction.
Note 2:
(a) Baseline is defined as the last observed value before the first dose of study medication.

TABLE 14
Analysis of Change from Baseline on the BACS Attention Cognition Domain by
Visit (Preliminary Analysis)

| | | | Compound | Compound |
|---|---|---|---|---|
| | | Compound (I) | (I) | (I) |
| Visit | Placebo | 50 mg | 125 mg | 500 mg |
| Statistic | (N = 87) | (N = 58) | (N = 56) | (N = 55) |
| Baseline (a) | | | | |
| N | 87 | 58 | 56 | 55 |
| Mean (SD) | 34.7 (12.64) | 34.2 (13.75) | 34.5 (11.35) | 34.1 (11.49) |
| Median | 36.0 | 35.5 | 33.5 | 35.0 |
| Minimum, Maximum | 6, 66 | −2, 59 | 13, 61 | 12, 63 |
| Week 6 | | | | |
| N | 79 | 56 | 56 | 51 |
| Mean (SD) | 35.2 (13.07) | 36.0 (14.78) | 36.7 (12.72) | 35.5 (12.68) |
| Median | 36.0 | 34.0 | 37.0 | 35.0 |
| Minimum, Maximum | 6, 64 | 5, 94 | 12, 65 | 13, 64 |
| Change from Baseline to Week | | | | |
| 6 (a) | | | | |
| N | 79 | 56 | 56 | 51 |
| Mean (SD) | 1.0 (5.58) | 0.9 (7.74) | 2.2 (6.07) | 0.9 (6.26) |
| Median | 2.0 | 0.0 | 2.0 | 0.0 |
| Minimum, Maximum | −17, 13 | −16, 38 | −8, 32 | −18, 15 |
| Adjusted Change from Baseline | | | | |
| to Week 6 | | | | |
| LS Mean (SE) | 1.0 (0.77) | 1.0 (0.90) | 2.2 (0.88) | 1.0 (0.96) |
| 95% CI | (−0.5, 2.6) | (−0.8, 2.7) | (0.5, 4.0) | (−0.9, 2.9) |
| Difference from Placebo for Week | | | | |
| 6 | | | | |
| LS Mean (SE) | | −0.1 (1.12) | 1.2 (1.12) | 0.0 (1.15) |
| 95% CI | | (−2.3, 2.1) | (−1.0, 3.4) | (−2.3, 2.2) |
| p-value (unadjusted) | | 0.524 | 0.146 | 0.516 |
| Week 12 | | | | |
| N | 71 | 51 | 50 | 45 |
| Mean (SD) | 36.7 (12.73) | 38.0 (11.83) | 36.2 (12.21) | 38.0 (14.08) |
| Median | 37.0 | 39.0 | 36.0 | 36.0 |
| Minimum, Maximum | 6, 67 | 7, 72 | 14, 69 | 8, 84 |
| Change from Baseline to Week | | | | |
| 12 (a) | | | | |
| N | 71 | 51 | 50 | 45 |
| Mean (SD) | 1.7 (6.40) | 2.2 (6.95) | 1.2 (6.41) | 3.1 (9.31) |
| Median | 2.0 | 2.0 | 2.0 | 2.0 |
| Minimum, Maximum | −12, 25 | −14, 21 | −13, 21 | −24, 39 |
| Adjusted Change from Baseline to | | | | |
| Week 12 | | | | |
| LS Mean (SE) | 1.9 (0.87) | 2.3 (1.02) | 1.4 (1.01) | 3.1 (1.09) |
| 95% CI | (0.2, 3.6) | (0.3, 4.3) | (−0.6, 3.4) | (1.0, 5.3) |
| Difference from Placebo for Week | | | | |
| 12 | | | | |
| LS Mean (SE) | | 0.4 (1.28) | −0.5 (1.29) | 1.2 (1.33) |
| 95% CI | | (−2.1, 2.9) | (−3.1, 2.0) | (−1.4, 3.8) |
| p-value (unadjusted) | | 0.376 | 0.659 | 0.178 |

CI—Confidence interval;
LS—Least squares;
SE—Standard error.
Note 1:
The change from Baseline on the BACS Attention Cognition Domain are analyzed using the mixed model for repeated measures (MMRM) with Baseline BACS Attention Cognition Domain as a covariate; pooled site, visit, treatment, and categorical age (randomization factor) as fixed factors; and treatment-by-visit and Baseline-by-visit interactions. Based on a Missing at Random Assumption, this analysis will be performed using observed case only. P-values are one-sided, with the alternative hypothesis that Compound (I) is superior to placebo in the clinically favorable direction.
Note 2:
(a) Baseline is defined as the last observed value before the first dose of study medication.

TABLE 15
Analysis of Change from Baseline on the BACS Executive Functions Cognition
Domain by Visit (Preliminary Analysis)

| | | | Compound | Compound |
|---|---|---|---|---|
| | | Compound (I) | (I) | (I) |
| Visit | Placebo | 50 mg | 125 mg | 500 mg |
| Statistic | (N = 87) | (N = 58) | (N = 56) | (N = 55) |
| Baseline (a) | | | | |
| N | 87 | 58 | 56 | 55 |
| Mean (SD) | 45.8 (15.63) | 42.7 (13.98) | 45.5 (13.88) | 45.9 (13.48) |
| Median | 49.0 | 45.5 | 47.5 | 50.0 |
| Minimum, Maximum | −21, 66 | −8, 65 | 1, 66 | 9, 68 |
| Week 6 | | | | |
| N | 79 | 56 | 56 | 51 |
| Mean (SD) | 45.0 (14.77) | 44.9 (11.66) | 46.3 (16.06) | 47.0 (10.95) |
| Median | 48.0 | 48.5 | 50.0 | 51.0 |
| Minimum, Maximum | −17, 66 | 4, 62 | −13, 69 | −3, 63 |
| Change from Baseline to Week | | | | |
| 6 (a) | | | | |
| N | 79 | 56 | 56 | 51 |
| Mean (SD) | −1.2 (9.79) | 1.6 (10.55) | 0.8 (9.01) | 1.3 (7.62) |
| Median | 0.0 | 2.0 | 0.0 | 0.0 |
| Minimum, Maximum | −48, 17 | −29, 60 | −32, 21 | −13, 20 |
| Adjusted Change from Baseline | | | | |
| to Week 6 | | | | |
| LS Mean (SE) | −1.2 (1.04) | 0.9 (1.22) | 0.8 (1.19) | 1.1 (1.29) |
| 95% CI | (−3.2, 0.9) | (−1.5, 3.3) | (−1.6, 3.1) | (−1.5, 3.6) |
| Difference from Placebo for Week | | | | |
| 6 | | | | |
| LS Mean (SE) | | 2.1 (1.53) | 2.0 (1.53) | 2.3 (1.57) |
| 95% CI | | (−0.9, 5.1) | (−1.0, 5.0) | (−0.8, 5.4) |
| p-value (unadjusted) | | 0.085 | 0.099 | 0.075 |
| Week 12 | | | | |
| N | 73 | 52 | 50 | 45 |
| Mean (SD) | 46.8 (12.21) | 46.4 (10.55) | 47.6 (13.17) | 48.5 (11.88) |
| Median | 49.0 | 49.5 | 51.0 | 51.0 |
| Minimum, Maximum | −4, 69 | 18, 63 | 11, 66 | 3, 68 |
| Change from Baseline to Week | | | | |
| 12 (a) | | | | |
| N | 73 | 52 | 50 | 45 |
| Mean (SD) | 0.5 (8.78) | 2.6 (7.73) | 2.0 (8.79) | 2.0 (9.28) |
| Median | 0.0 | 1.0 | 3.0 | 0.0 |
| Minimum, Maximum | −32, 31 | −14, 34 | −33, 17 | −17, 26 |
| Adjusted Change from Baseline to | | | | |
| Week 12 | | | | |
| LS Mean (SE) | 0.6 (0.94) | 1.8 (1.09) | 2.0 (1.09) | 2.0 (1.19) |
| 95% CI | (−1.3, 2.4) | (−0.4, 4.0) | (−0.2, 4.1) | (−0.3, 4.3) |
| Difference from Placebo for Week | | | | |
| 12 | | | | |
| LS Mean (SE) | | 1.2 (1.36) | 1.4 (1.38) | 1.4 (1.42) |
| 95% CI | | (−1.5, 3.9) | (−1.3, 4.1) | (−1.4, 4.2) |
| p-value (unadjusted) | | 0.188 | 0.157 | 0.160 |

CI—Confidence interval;
LS—Least squares;
SE—Standard error.
Note 1:
The change from Baseline on the BACS Executive Functions Cognition Domain are analyzed using the mixed model for repeated measures (MMRM) with Baseline BACS Executive Functions Cognition Domain as a covariate; pooled site, visit, treatment, and categorical age (randomization factor) as fixed factors; and treatment-by-visit and Baseline-by-visit interactions. Based on a Missing at Random Assumption, this analysis will be performed using observed case only. P-values are one-sided, with the alternative hypothesis that Compound (I) is superior to placebo in the clinically favorable direction.
Note 2:
(a) Baseline is defined as the last observed value before the first dose of study medication.

TABLE 16
Analysis of Change from Baseline on the BACS Verbal Fluency Cognition
Domain by Visit (Preliminary Analysis)

| | | | Compound | Compound |
|---|---|---|---|---|
| | | Compound (I) | (I) | (I) |
| Visit | Placebo | 50 mg | 125 mg | 500 mg |
| Statistic | (N = 87) | (N = 58) | (N = 56) | (N = 55) |
| Baseline (a) | | | | |
| N | 87 | 57 | 56 | 55 |
| Mean (SD) | 38.0 (10.89) | 39.7 (9.78) | 38.1 (8.46) | 38.7 (9.45) |
| Median | 38.0 | 39.0 | 38.0 | 38.0 |
| Minimum, Maximum | 14, 69 | 19, 61 | 21, 53 | 22, 67 |
| Week 6 | | | | |
| N | 79 | 55 | 55 | 51 |
| Mean (SD) | 38.1 (10.88) | 39.9 (10.86) | 39.1 (8.98) | 39.6 (10.14) |
| Median | 37.0 | 38.0 | 40.0 | 40.0 |
| Minimum, Maximum | 16, 65 | 16, 71 | 19, 58 | 20, 63 |
| Change from Baseline to Week | | | | |
| 6 (a) | | | | |
| N | 79 | 55 | 55 | 51 |
| Mean (SD) | 0.5 (4.22) | −0.2 (5.57) | 0.8 (4.53) | 0.8 (5.87) |
| Median | 1.0 | 0.0 | 1.0 | 1.0 |
| Minimum, Maximum | −11, 12 | −22, 13 | −13, 11 | −17, 15 |
| Adjusted Change from Baseline | | | | |
| to Week 6 | | | | |
| LS Mean (SE) | 0.2 (0.60) | −0.3 (0.70) | 0.7 (0.68) | 0.5 (0.74) |
| 95% CI | (−1.0, 1.3) | (−1.7, 1.0) | (−0.6, 2.1) | (−1.0, 1.9) |
| Difference from Placebo for Week | | | | |
| 6 | | | | |
| LS Mean (SE) | | −0.5 (0.87) | 0.6 (0.87) | 0.3 (0.89) |
| 95% CI | | (−2.2, 1.2) | (−1.1, 2.3) | (−1.4, 2.1) |
| p-value (unadjusted) | | 0.722 | 0.258 | 0.364 |
| Week 12 | | | | |
| N | 73 | 51 | 50 | 45 |
| Mean (SD) | 38.9 (11.91) | 41.8 (10.41) | 40.6 (9.54) | 40.6 (10.30) |
| Median | 38.0 | 40.0 | 39.0 | 40.0 |
| Minimum, Maximum | 13, 77 | 24, 70 | 21, 62 | 20, 63 |
| Change from Baseline to Week | | | | |
| 12 (a) | | | | |
| N | 73 | 51 | 50 | 45 |
| Mean (SD) | 1.2 (5.53) | 1.5 (5.87) | 2.1 (5.59) | 1.3 (6.95) |
| Median | 0.0 | 0.0 | 2.0 | 0.0 |
| Minimum, Maximum | −11, 17 | −12, 14 | −11, 14 | −11, 20 |
| Adjusted Change from Baseline to | | | | |
| Week 12 | | | | |
| LS Mean (SE) | 1.0 (0.72) | 1.2 (0.85) | 1.9 (0.84) | 0.5 (0.91) |
| 95% CI | (−0.4, 2.4) | (−0.5, 2.9) | (0.2, 3.6) | (−1.3, 2.3) |
| Difference from Placebo for Week | | | | |
| 12 | | | | |
| LS Mean (SE) | | 0.2 (1.07) | 0.9 (1.07) | −0.5 (1.11) |
| 95% CI | | (−1.9, 2.3) | (−1.2, 3.0) | (−2.7, 1.7) |
| p-value (unadjusted) | | 0.433 | 0.202 | 0.668 |

CI—Confidence interval;
LS—Least squares;
SE—Standard error.
Note 1:
The change from Baseline on the BACS Verbal Fluency Cognition Domain are analyzed using the mixed model for repeated measures (MMRM) with Baseline BACS Verbal Fluency Cognition Domain as a covariate; pooled site, visit, treatment, and categorical age (randomization factor) as fixed factors; and treatment-by-visit and Baseline-by-visit interactions. Based on a Missing at Random Assumption, this analysis will be performed using observed case only. P-values are one-sided, with the alternative hypothesis that Compound (I) is superior to placebo in the clinically favorable direction.
Note 2:
(a) Baseline is defined as the last observed value before the first dose of study medication.

TABLE 17
Analysis of Change from Baseline on the SCoRS Interviewer Total Score at Week
12 (Preliminary Analysis)

| | | Compound (I) | Compound (I) | Compound (I) |
|---|---|---|---|---|
| | Placebo | 50 mg | 125 mg | 500 mg |
| Visit Statistic | (N = 87) | (N = 58) | (N = 56) | (N = 55) |
| Baseline (a) | | | | |
| N | 84 | 57 | 55 | 52 |
| Mean (SD) | 39.7 (9.96) | 39.6 (10.73) | 38.5 (10.29) | 41.6 (9.98) |
| Median | 39.0 | 40.0 | 39.0 | 40.5 |
| Minimum, Maximum | 20, 61 | 21, 73 | 20, 62 | 23, 60 |
| Week 12 | | | | |
| N | 77 | 54 | 52 | 49 |
| Mean (SD) | 37.8 (9.39) | 35.5 (10.91) | 36.1 (9.69) | 39.4 (9.60) |
| Median | 38.0 | 32.0 | 36.5 | 39.0 |
| Minimum, Maximum | 20, 58 | 20, 72 | 20, 57 | 21, 59 |
| Change from Baseline to | | | | |
| Week 12 (a) | | | | |
| N | 74 | 53 | 51 | 47 |
| Mean (SD) | −1.8 (5.14) | −4.0 (5.93) | −2.1 (5.89) | −2.4 (5.34) |
| Median | −1.0 | −3.0 | −2.0 | −2.0 |
| Minimum, Maximum | −16, 14 | −20, 17 | −16, 15 | −15, 15 |
| Adjusted Change from | | | | |
| Baseline to Week 12 | | | | |
| LS Mean (SE) | −1.4 (0.66) | −3.7 (0.77) | −2.1 (0.77) | −1.5 (0.84) |
| 95% CI | (−2.7, −0.1) | (−5.2, −2.2) | (−3.6, −0.6) | (−3.2, 0.1) |
| Difference from Placebo for | | | | |
| Week 12 | | | | |
| LS Mean (SE) | | −2.4 (0.94) | −0.7 (0.96) | −0.1 (0.98) |
| 95% CI | | (−4.2, −0.5) | (−2.6, 1.2) | (−2.1, 1.8) |
| p-value (unadjusted) | | 0.007* | 0.236 | 0.449 |

Note 1:
The change from Baseline on the SCoRS Interviewer Total Score is analyzed using the mixed model for repeated measures (MMRM) with Baseline SCoRS Interviewer Total Score as a covariate; pooled site, visit, treatment, and categorical age (randomization factor) as fixed factors; and treatment-by-visit and Baseline-by-visit interactions. Based on a Missing at Random Assumption, this analysis will be performed using observed case only. P-values are one-sided, with the alternative hypothesis that Compound (I) is superior to placebo in the clinically favorable direction.
Note 2:
(a) Baseline is defined as the last observed value before the first dose of study medication.
*indicates statistical significance at the 0.025 level (one-sided).

### Example 7: A Study to Evaluate Efficacy, Tolerability, Pharmacodynamics, and Pharmacokinetics of Multiple Oral Dose Levels of Compound (I) in Adults with Schizophrenia (NCT03359785)

NCT03359785 was a randomized, double-blind, placebo-controlled cross-over Phase 1b clinical trial in schizophrenia patients at a single site to evaluate PD effects, safety, tolerability, and pharmacokinetics of multiple oral doses of Compound (I). The objective of the study was to assess pharmacodynamic (PD) biomarkers of Compound (I) on measures related to cerebellar circuitry function and NMDA pharmacology that are known to be affected in schizophrenia.

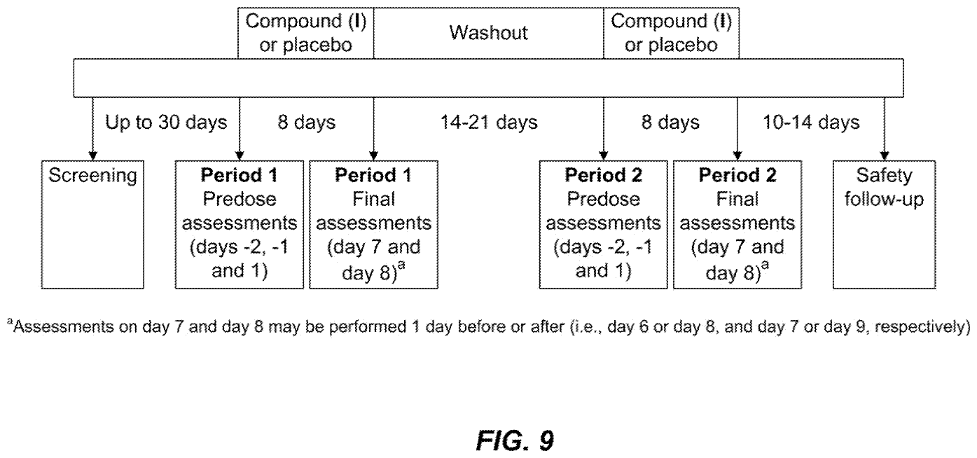

The study had a two-sequence, two-way crossover design, in which participants received either 50 or 500 mg of Compound (I) and placebo for 8 consecutive days in two periods separated by a 2- or 3-week washout (FIG. 9).

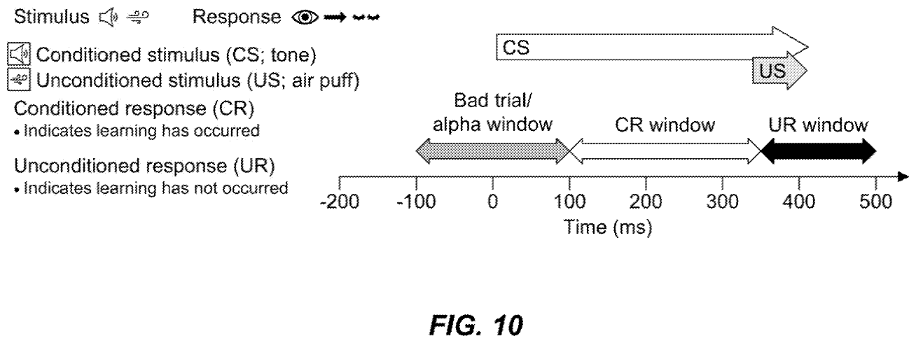

The primary endpoint was the average percentage of conditioned responses during the eye-blink conditioning (EBC) test at day 8 of each treatment period (FIG. 10). EBC was used to assess the impact of Compound (I) on cerebellar circuitry function. EBC is a highly conserved reflex that is dependent on cerebellar circuitry, with NMDA receptors playing a key role. EBC is impaired in patients with schizophrenia compared with healthy controls. Compound (I) has shown robust effects in reversing scopolamine-induced EBC deficits in preclinical studies (see, e.g., Example 2 of WO 2015/132608).

Evoked potentials in the electroencephalogram were also tested to assess the impact of multiple oral doses of Compound (I) on NMDA-sensitive physiological responses that are affected in schizophrenia. Specifically, mismatch negativity (MMN), auditory steady state response (ASSR), and P300 were measured at baseline and following 8 days of treatment in both periods.

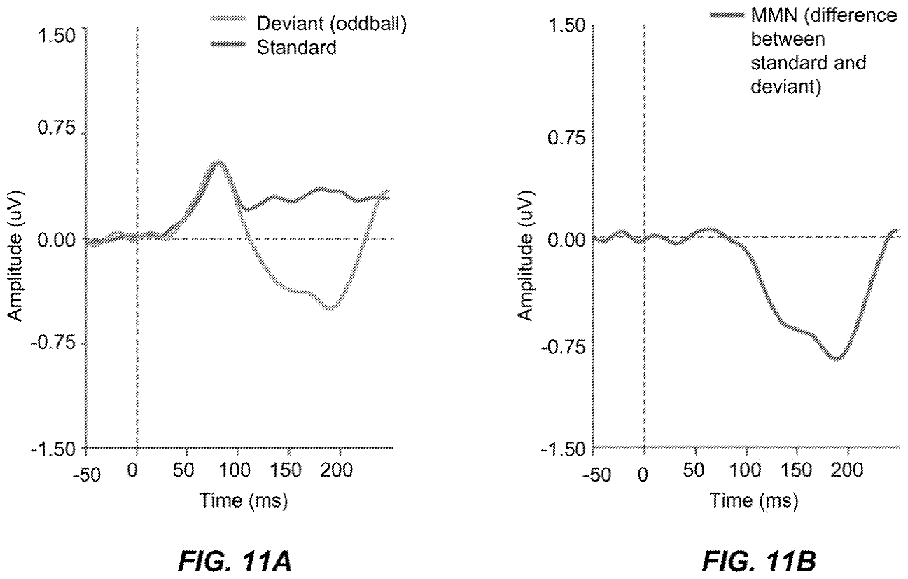

MMN is an oddball event-related potential sensitive to NMDA-receptor pharmacology. MMN is an event related potential (ERP) evoked in response to unattended changes in background stimulation and is responsive to D-serine elevations. There is evidence of D-serine administration improving MMN in patients with schizophrenia. MMN reflects an automatic process of detecting a mismatch between a deviant stimulus and a sensory-memory trace (FIGS. 11A, 11B). Smaller amplitudes of MMN have been consistently identified in schizophrenia participants, with a large effect size compared with healthy controls. In this study, MMN amplitude was measured at Midline frontal electrode (Fz) of electroencephalogram [EEG].

The P300 wave is an ERP component that is elicited by the presentation of a novel, behaviorally relevant target stimulus embedded among irrelevant stimuli in a manner similar to MMN, but requiring active listening and responding from participants. Auditory stimuli were presented in an oddball paradigm consisting of 1 standard tone and 1 target tone. Participants were instructed to push a button as quickly as possible when they hear the target tone, but not when they hear the standard tone. P300 reflects allocation of attention and activation of immediate memory. P300 amplitude indexes brain actions when the mental representation of the stimulus environment is updated, while P300 latency indexes stimulus classification speed unrelated to response selection processes. P300 amplitude was measured at midline parietal electrode (Pz) of EEG.

ASSR are evoked oscillatory responses that are entrained to the frequency and phase of temporally modulated stimuli. Individuals with schizophrenia experience subjective sensory anomalies and objective deficits on assessment of sensory function. These deficits can be produced by abnormal signaling in the sensory pathways and sensory cortex or by later-stage disturbances in the cognitive processing of such inputs. ASSR can be used to assess the integrity of sensory pathways including cortical processing. ASSR was measured at midline central electrode (Cz) of EEG.

Secondary endpoints of the study included: (1) mean mismatch negativity (MMN) amplitude at day 8 of each treatment period; (2) mean Auditory Steady State Response (ASSR) at day 8 of each treatment period; and (3) Brief Assessment of Cognition in Schizophrenia (BACS) score at day 7 of each treatment period.

Levels of D- and L-serine were also analyzed for all groups. Specifically, other secondary outcome measures included: (1) mean concentration of plasma D-serine and L-serine levels at day 8; (2) mean plasma D-serine to total serine ratio at day 8; and (3) mean plasma concentration of Compound (I).

Safety objectives included the incidence of treatment-emergent adverse events (TEAEs), laboratory tests, vital signs, and electrocardiogram (ECG) assessments.

All endpoints were analyzed as change from baseline compared to placebo within the same subjects. Statistical comparisons were conducted with an ANOVA model, with treatment sequence, period, and treatment as fixed effects, and subject as a random effect. As a supplementary analysis, an analysis of covariance model (ANCOVA) was applied to the primary endpoint with (period) baseline as a covariate, treatment sequence, period, treatment effect and baseline-by-treatment as fixed effects, and subject as a random effect.

COVID-19 impacted the study, resulting in fewer completers than initially planned, which affected the power of the study. Therefore, the data are considered exploratory in nature, and the focus of the study was the directionality of change and effect sizes.

The Phase 1b study enrolled 31 patients, aged 18 to 60 years. Participants were symptomatically stabilized patients with schizophrenia, receiving stable antipsychotic medication over 2 months. 12 patients completed both active and placebo periods for each dose. Patients were primarily male (26/31) and African-American (26/31). 14 patients were randomized to Compound (I) 50 mg (median age, 45 years; 11 men), and 12 completed the study. 17 patients were randomized to Compound (I) 500 mg (median age, 34 years; 15 men), and 12 completed the study.

Inclusion criteria for the study included:
1. Have a body mass index (BMI) greater than or equal to (>=) 18.5 and less than or equal to (<=) 40.0 (kilogram per square meter [kg/m\hat{ }2]) at the Screening Visit.
2. With a current Diagnostic and Statistical Manual of Mental Disorders, Fifth Edition (DSM-5) diagnosis of schizophrenia who are receiving stable antipsychotic therapy (no increase, no decrease greater than [>] 20% in dose in the preceding 2 months).
3. Positive and Negative Syndrome Scale (PANSS) negative symptom factor score (NSFS)>=15, stable screening and baseline PANSS NSFS (<25% change).
4. PANSS total score <=90; stable screening and baseline PANSS total score (less than [<] 20% change).
5. Receiving stable (no increase, no decrease >25% in dose in the preceding 2 months) antipsychotic medication at doses not to exceed risperidone 6 mg or its equivalent. Concomitant treatment with a subtherapeutic dose of a second antipsychotic may be permitted with sponsor or designee approval if used as a hypnotic (maximum of quetiapine 300 mg or its equivalent once daily at bedtime) and subject does not show morning sedation as per the investigator opinion, but not if it is used for refractory positive psychosis symptoms. Under this exception, the total daily dose the second antipsychotic will not have to be included in the calculation of the 6 mg/day risperidone-equivalent limit.

Exclusion criteria for the study included:
1. Has a history of cancer (malignancy) excluding treated basal cell carcinoma or treated stage 0 (in situ) cervical carcinoma.
2. Has a history of significant multiple and/or severe allergies (example [eg], food, drug, latex allergy) or has had an anaphylactic reaction or significant intolerability to prescription or nonprescription drugs or food.
3. Has a QT interval with Fridericia's correction method (QTcF)>450 milliseconds [ms] (males) or >470 ms (females) confirmed with one repeat testing, at the Screening Visit or Check-in.
4. Has a positive alcohol or drug screen for disallowed substances, including amphetamines, barbiturates, cocaine, marijuana, methadone, methamphetamine, 3,4-methylenedioxymethamphetamine, phencyclidine, or nonprescribed benzodiazepines or opiates.
5. Is positive for hepatitis B surface antigen (HBsAg), hepatitis C antibodies, or has HIV by history (confirmatory testing is allowed; most sensitive test should take precedence).
6. Had major surgery, donated or lost 250 milliliter [mL] of blood within 4 weeks prior to the prestudy (screening) visit.
7. Has a known hypersensitivity to any component of the formulation of Compound (I).
8. Has a history of significant skin reactions (hypersensitivity) to adhesives, metals or plastic.
9. Is considered by the investigator to be at imminent risk of suicide or injury to self, others, or property, or participants who within the past year prior to Screening have attempted suicide. Participants who have positive answers on item 4 or 5 on the Columbia Suicide Severity Rating Scale (C-SSRS) (based on the past year) prior to randomization are excluded.

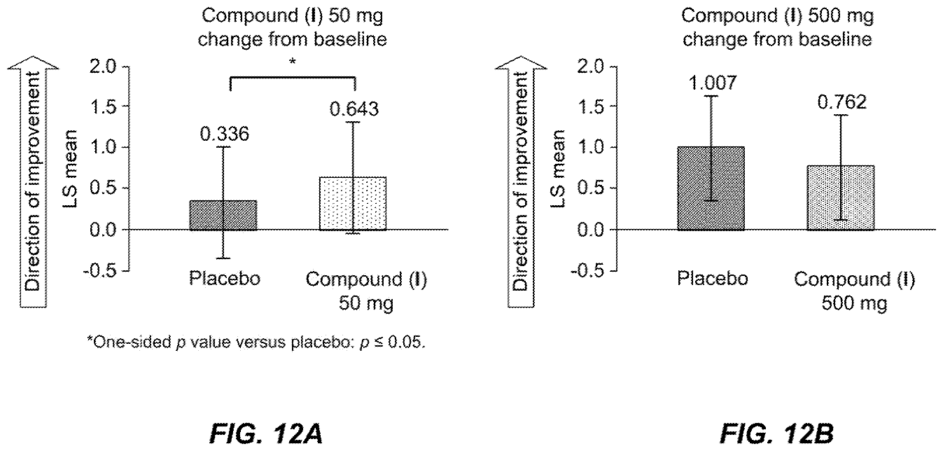

The primary endpoint was change from baseline in EBC. A trend toward improvement compared with placebo was observed with Compound (I) 50 mg (p=0.109), but not with Compound (I) 500 mg (p=0.777) (FIGS. 12A, 12B). The effect sizes were 0.246 and −0.198 for Compound (I) 50 mg and Compound (I) 500 mg, respectively (ANCOVA).

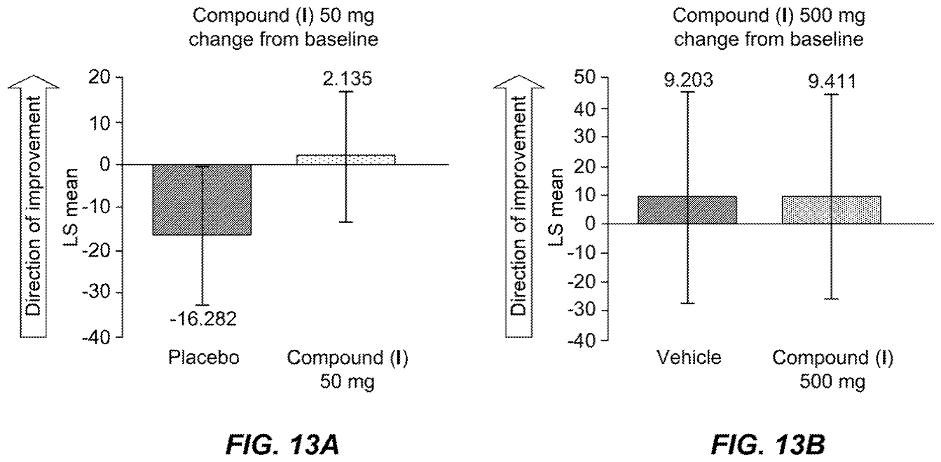

On secondary endpoints, the MMN amplitude improved (became more negative than baseline) with Compound (I) 50 mg, but not with Compound (I) 500 mg, compared to placebo (FIGS. 13A, 13B). The difference between 50 mg and placebo in LS means change from baseline was statistically significant (50 mg: −0.239 (−0.863 to 0.386); placebo: 0.669 (0.012 to 1.326); p=0.0497; effect size: −0.691). The 500 mg dose did not show a significant difference from placebo in change from baseline (500 mg: 0.594 (−0.245 to 1.432); placebo: −0.154 (−1.008 to 0.700); p=0.8517; effect size: 0.389).

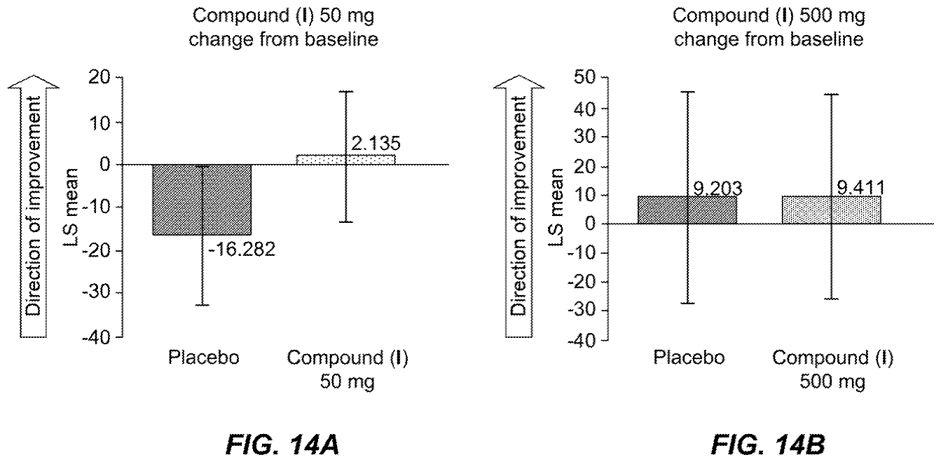

The ASSR gamma band power showed numerical increase from baseline with Compound (I) 50 mg but not Compound (I) 500 mg compared to placebo (FIGS. 14A, 14B). The 50 mg dose showed a greater change from baseline than placebo in ASSR gamma power, but it showed only a trend towards significance (50 mg: 2.135 (−13.127 to 17.396); placebo: −16.282 (−32.353 to −0.211); p=0.056; effect size: 0.575). The 500 mg dose change from baseline was similar to placebo (500 mg: 9.411 (−26.018 to 44.480); placebo: 9.203 (−26.981 to 45.387); p=0.495; effect size: 0.003).

Compound (I) was generally well tolerated. There were no deaths, serious adverse events (AEs), or TEAEs leading to discontinuation of study drug. Additionally, there were no significant findings in safety, laboratory, or ECG assessments. Of the five adverse events that were reported, all were mild and not considered to be related to the study drug, and all patients recovered.

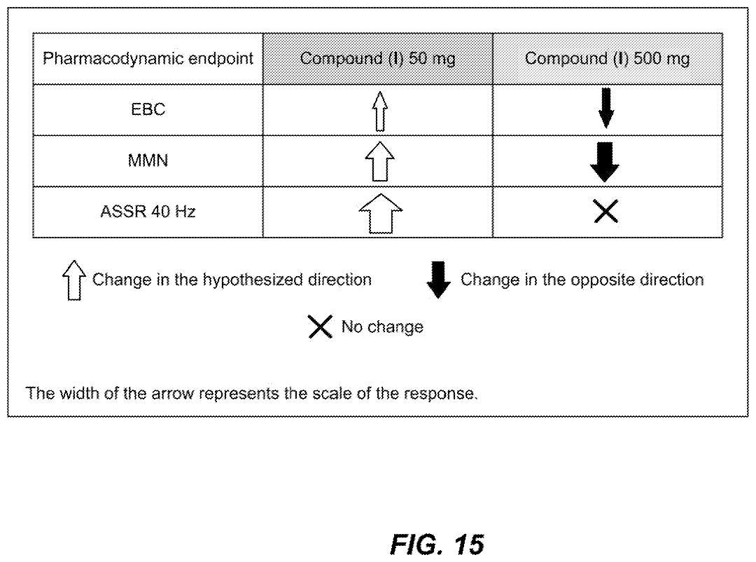

In summary, Compound (I) demonstrated a statistically significant improvement in MMN amplitude and a numerical improvement in ASSR gamma band power in stable patients with schizophrenia (FIG. 15). These effects were observed only at the lower dose of 50 mg and not with 500 mg. These results support the findings from the recently completed phase 2 INTERACT study (ClinicalTrials.gov: NCT03382639) that showed improvements in BACS and Schizophrenia Cognition Rating Scale (SCORS) in patients with schizophrenia taking Compound (I) 50 mg for 12 weeks, but not in those taking Compound (I) 500 mg. The observed higher impact of the lower dose (potential inverted U-shaped dose response) is consistent with preclinical data with Compound (I) (e.g., preclinical studies with Compound (I) on long-term potentiation in mice) and with NMDA receptor pharmacology in general. Although the effects on the primary EBC endpoint were not significant, this may be due to the sample size being underpowered for this assay. Overall, the data suggest low doses of Compound (I) improve the outcome of neural circuitry activity that has been associated with NMDA receptor pharmacology and correlated with cognitive performance in schizophrenia. Additionally, in combination with the INTERACT study findings, this study suggests that MMN is a potential biomarker for NMDA pharmacology that predicts cognitive improvement with more extended treatment.

### Example 8: A Randomized, Double-Blind, Placebo-Controlled, Parallel-Group Study to Evaluate the Efficacy, Safety, and Tolerability of Compound (I) in Subjects with Cognitive Impairment Associated with Schizophrenia, Followed by Open-Label Treatment

A Phase 2b, randomized, double-blind, parallel, placebo-controlled study with a 12-month open-label extension will be conducted to evaluate the efficacy, safety, and tolerability, and pharmacokinetics (PK) of treatment with Compound (I) when administered orally once daily as an adjunctive treatment on improving symptoms of cognitive impairment associated with schizophrenia (CIAS). Additionally, the study will evaluate the long-term safety and tolerability of treatment with 50 mg Compound (I) QD.

The Phase 2b study will be conducted at approximately 45 study centers in regions including, but not limited to, North America and Europe and will enroll approximately 308 adult subjects with schizophrenia. The expected duration of study participation for each subject is approximately 72 weeks, including 4 weeks of screening, 14 weeks of double blind treatment, 52 weeks of open-label treatment, and 2 weeks of follow-up. In order to obtain, as objectively as possible, assessment of symptom severity during treatment with Compound (I), investigators and staff interacting with the investigators will be blinded to the following: timing of randomization; timing of endpoint assessment; and details of the statistical analysis methodology.

The study will enroll male and female patients between the ages of 18 and 50 years old with schizophrenia as defined by the MINI Version 7.0.2, where the patient's initial diagnosis of schizophrenia must have occurred more than one year before screening (Visit 1). Subjects must have stable symptomatology for at least 3 months before screening and must currently be receiving a stable regimen of psychotropic medications with no clinically meaningful change (no increase in dose, <25% decrease in dose for tolerability) in the prescribed dose for at least 2 months before screening. In addition, subjects must have limited PANSS symptoms.

Subjects must meet all of the following inclusion criteria:
1. Completed written informed consent.
2. Subject must be 18 to 50 years of age (inclusive) and able to comply with all protocol procedures.
3. Diagnosis of schizophrenia as defined by the Diagnostic and Statistical Manual of Mental Disorders (DSM-5).
4. The initial diagnosis of schizophrenia must be ≥1 year before screening.
5. The subject is currently receiving a stable regimen of psychotropic medications.
6. Subject has stable symptomatology >3 months before the screening visit.
7. The subject must have an adult informant.
8. A body weight of at least 45 kg and a body mass index (BMI) of 18.0 to 40.5 kg/m², inclusive.

Additionally, subjects will be excluded from the study if they meet any of the following criteria:
1. Pregnant or breastfeeding or plans to become pregnant during the study.
2. Exhibit more than a minimal level of extrapyramidal signs/symptoms.
3. Schizophrenia diagnosis occurred before 12 years of age.
4. Lifetime diagnosis of schizoaffective disorder, bipolar disorder, or obsessive-compulsive disorder.
5. Recent occurrence of panic disorder, depressive episode, or other comorbid psychiatric conditions.
6. Considered by the investigator to be at imminent risk of suicide or injury to self, others, or property, or the subject has attempted suicide within 6 months before screening.
7. Diagnosis of moderate or severe substance use disorder (with the exception of nicotine dependence) within 12 months prior to screening.
8. Positive drug screen for disallowed substances.
9. Any other medical or psychiatric condition or cognitive impairment that may interfere with study conduct or clinical assessments.

### Screening Period (Days −28 to −1)

At the Screening Visit, subjects who provide informed consent and meet a current diagnosis of schizophrenia, as defined by the Mini International Neuropsychiatric Interview (MINI) Version 7.0.2, will undergo additional screening assessments to determine eligibility. In addition, the adult informant must provide written informed consent and participate in at least one screening interview at the study site during the Screening Period (Days −28 to −1). Adherence to background daily oral antipsychotic medication will be assessed throughout the Screening Period (Days 28 to 1) using a medication adherence application; subjects using a long-acting injectable antipsychotic will not use the medication adherence application during the screening period.

### Double-Blind Treatment Period (Days 1 to 98)

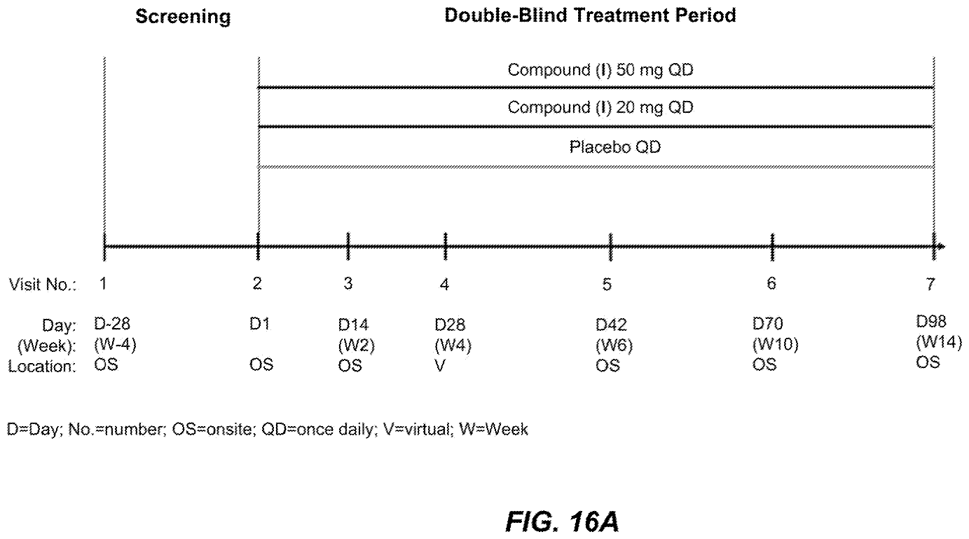

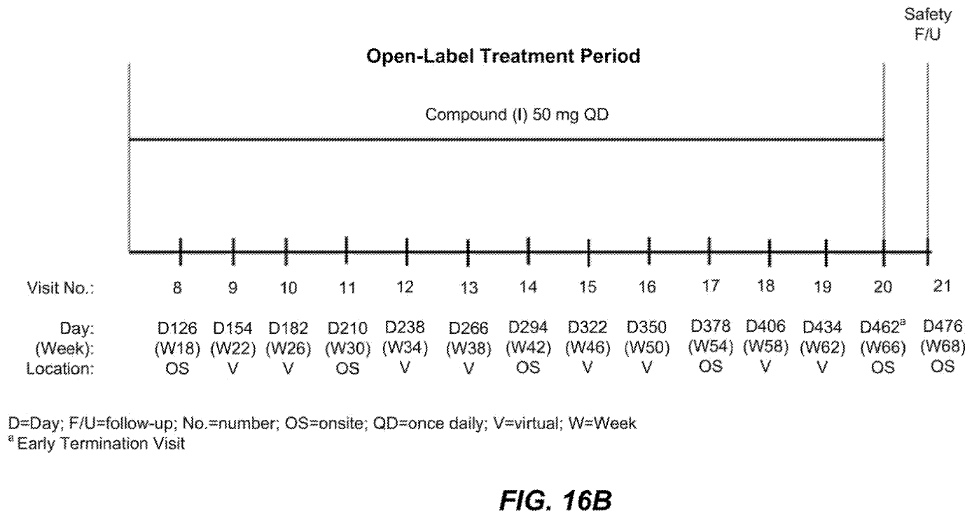

Eligible subjects will be randomized (1:1:2) to receive Compound (I) 20 mg, Compound (I) 50 mg, or placebo orally QD during the 14 week Double Blind Treatment Period. Subsequently, all subjects who complete the Double Blind Treatment Period and who are continuing to take study treatment will enter the 12 month Open Label Treatment Period and receive Compound (I) 50 mg QD. A final Safety Follow-Up Visit will be conducted approximately 2 weeks after the final dose of study treatment (Day 476 [Visit 21]). The study design schematics are provided in FIGS. 16A (screening and double-blind treatment periods) and 16B (open-label treatment and safety follow-up periods).

Subjects will be instructed to take two tablets QD in the morning with water or milk and to avoid drinking juices 1 hour before and 1 hour after taking study treatment. Compound (I) will be supplied as matching tablet dosage forms of 10 and 25 mg for oral administration. Adherence to study treatment will be monitored with a medication adherence application.

Subjects will have onsite and virtual study visits. Efforts will be made to perform cognitive and cognitive functioning assessments at approximately the same time of day and using the same rater throughout the Double-Blind Treatment Period (Days 1 to 98).

Adherence to background daily oral antipsychotic medication will continue to be assessed throughout the Double Blind Treatment Period (Days 1 to 98); subjects using a long-acting injectable antipsychotic will use the medication adherence application to record study treatment intake only.

### Open-Label Treatment Period (Days 99 to 462)

After Day 98 (Visit 7) assessments have been performed, all subjects who are continuing to take study treatment will enter the 12-month Open Label Treatment Period and receive Compound (I) 50 mg QD. Dose reductions or adjustments will not be permitted during this period. Subjects will be instructed to take two tablets (25 mg each) QD starting on the morning of Day 99. As in the Double-Blind Treatment Period, subjects will be instructed to take both tablets in the morning with water or milk and to avoid drinking juices 1 hour before and 1 hour after taking study treatment. Subjects will have onsite and virtual study visits.

A final Safety Follow-Up Visit will be conducted 14 days after each subject's final dose of study treatment (Day 476 [Visit 21]).

### Efficacy Endpoints

The primary endpoint for the study will be the change from baseline on the Brief Assessment of Cognition in Schizophrenia (BACS) composite score at Day 98.

The key secondary endpoint will be change from baseline on the Schizophrenia Cognition Rating Scale (SCORS) interviewer score at Day 98.

Additional secondary endpoints will include: (1) change from baseline on the Continuous Performance Test-Identical Pairs (CPT-IP) test at Day 98; (2) change from baseline on the Brief Visuospatial Memory Test-Revised (BVMT-R) test at Day 98; (3) change from baseline on the Mayer-Salovey-Caruso Emotional Intelligence Test (MSCEIT) at Day 98; (4) change from baseline on the Virtual Reality Functional Capacity Assessment Tool (VRFCAT) at Day 98; and (5) change from baseline on the Clinical Global Impression-Severity Scale (CGI-S) score at Day 98. The BACS is a cognition assessment battery that assesses the following 6 domains of cognitive function found to be consistently impaired in schizophrenia: verbal memory, processing speed, working memory, verbal fluency, motor function, and executive function. It is scientifically validated and has high reliability and sensitivity to impairment (Keefe et al., 2004). The BACS evaluates aspects of function that are related to cognitive improvement, and its global score can be used to assess overall cognitive function. See, e.g., Bralet M C, Navarre M, Eskenazi A M, Lucas-Ross M, Falissard B. Intérêt d'un nouvel instrument dans l'évaluation cognitive dans la schizophrénie [Interest of a new instrument to assess cognition in schizophrenia: The Brief Assessment of Cognition in Schizophrenia (BACS)]. Encephale. 2008;34 (6): 557-62. French. The BACS assessment is devised for easy administration and scoring by nonpsychologists. It is specifically designed to measure treatment-related improvements in cognition, is available in multiple languages, and has a large database of normative data available for generating standardized scores. Originally developed as a pen and paper assessment, a tablet-based version of the BACS (BAC App) has been developed and validated against the original version (see, e.g., Atkins A S, Tseng T, Vaughan A, et al. Validation of the tablet-administered Brief Assessment of Cognition (BAC App). Schizophr Res. 2017; 181:100-6) and employs automated response capture and scoring. The BACS assessment takes approximately 35 minutes to complete. The BACS will be administered and scored by the investigator or other qualified site personnel throughout the Double Blind Treatment Period (Days 1 to 98).

The Schizophrenia Cognition Rating Scale (SCORS) interviewer score is a scientifically validated interview-based measure of cognitive functioning. It was developed to specifically assess aspects of cognitive functioning found in each of the 7 cognitive domains assessed by the MATRICS Consensus Cognitive Battery (MCCB) which is a primary outcome measure for clinical studies of new medications to improve cognition in schizophrenia. See, e.g., Keefe R S, Poe M, Walker T M, Kang J W, Harvey P D. “The Schizophrenia Cognition Rating Scale: an interview-based assessment and its relationship to cognition, real-world functioning, and functional capacity.” Am J Psychiatry. 2006;163 (3): 426-32. The scale is intended to incorporate information obtained from an informant who bases his/her responses on interaction with and knowledge of the subject. The SCORS includes 20 items focusing on cognitive impairment and the degree to which it affects day-to-day functioning, as well as a global functioning scale; at follow-up visits there is also a global scale reflecting change from the beginning of the subject's treatment rated by the administrator, the informant, and the subject. The SCORS assessment takes approximately 12 minutes to complete. SCORS will be administered and scored by the investigator or other qualified site personnel throughout the Double Blind Treatment Period (Days 1 to 98).

The Continuous Performance Test-Identical Pairs (CPT-IP) test evaluates sustained attention and vigilance. See, e.g., Keilp J G, Herrera J, Stritzke P, Cornblatt B A. “The continuous performance test, identical pairs version (CPT-IP): III. Brain functioning during performance of numbers and shapes subtasks.” Psychiatry Res. 1997;74 (1): 35-45. This test is a variant of the original CPT and was designed to be more cognitively challenging than the original. In this computerized test, 150 stimuli (4-digit numbers) will be rapidly flashed in sequence; subjects will be asked to press a response button whenever 2 identical stimuli appear in a row. In every test, there are 30 identical pairs and an equal number of “catch” trials (pairs of very similar, but not identical, stimuli). The remaining 90 stimuli are dissimilar and randomly organized. This version of the CPT-IP includes a series of practice sequences, which will initially use 2-digit numbers as stimuli but will then progress to using 4-digit numbers, which require more short-term memory to perform correctly. The CPT-IP is demanding of attention and working memory, since each stimulus must first be processed, then retained in working memory until the next one appears, and a comparison can be made. The CPT-IP assessment takes approximately 10 to 20 minutes to complete. CPT-IP will be administered and scored by the investigator or other qualified site personnel throughout the Double Blind Treatment Period (Days 1 to 98).

The Brief Visuospatial Memory Test-Revised (BVMT-R) test is a reliable assessment of visual learning and memory that was designed to be used as part of a larger neuropsychological battery and to document changes over time. See, e.g., Tam J W, Schmitter-Edgecombe M. “The role of processing speed in the Brief Visuospatial Memory Test-revised.” Clin. Neuropsychol. 2013;27 (6): 962-72. In this test, subjects will be asked to reproduce 6 geometric figures (printed in a 2×3 array) from memory. A series of trials will be conducted. During the Learning Trial, the subject views the stimulus page for 10 seconds, and is then asked to reproduce as many of the arrays as possible in the correct location on a page in the response booklet. A Delayed Recall Trial then is administered similarly but uses a 25-minute delay. In the Recognition Trial, the subject will be asked to identify which of 12 arrays were included among the original geometric figures. The BVMT-R assessment takes approximately 45 minutes (including a 25-minute delay) to complete. BVMT-R will be administered and scored by the investigator or other qualified site personnel throughout the Double Blind Treatment Period (Days 1 to 98).

The Mayer-Salovey-Caruso Emotional Intelligence Test (MSCEIT) tests emotional intelligence by evaluating how well subjects manage their emotions in everyday situations. The MSCEIT was designed to measure the 4 branches of Mayer and Solvey's model of Emotional Intelligence: Perceiving Emotions, Facilitating Thought, Understanding Emotions, and Managing Emotions. See, e.g., Mayer J D, Salovey P, Caruso D. “Mayer-Salovey-Caruso Emotional Intelligence Test (MSCEIT) Users Manual.” Toronto, Ontario: Multi-Health Systems. 2002. This computerized test consists of 141 items. MSCEIT generates 15 main scores (Total Emotional Intelligence score, 2 Area scores, 4 Branch scores, and 8 Task scores) and 3 Supplemental scores. The MSCEIT assessment takes approximately 30 to 45 minutes to complete. The MSCEIT will be administered and scored by the investigator or other qualified site personnel throughout the Double Blind Treatment Period (Days 1 to 98).

The Virtual Reality Functional Capacity Assessment Tool (VRFCAT) is an immersive, virtual-reality-based computerized assessment that evaluates functional capacity across the following 4 domains: transportation, finances, household management, and planning. Briefly, the VRFCAT consists of 4 mini scenarios, which include checking the kitchen for the availability of items to complete a recipe and planning a trip to the grocery store, taking a bus and paying the correct fare, shopping for the items in a store, and returning home. The VRFCAT is a reliable and validated instrument and has demonstrated sensitivity to basic functional capacity deficits in patients with schizophrenia. See, e.g., Keefe R S E, Davis V G, Atkins A S, et al. “Validation of a computerized test of functional capacity.” Schizophr. Res. 2016;175 (1-3): 90-6. The VRFCAT assessment takes approximately 20 minutes to complete.

The “Clinical Global Impression of Severity” or “CGI-S” is a 7-point scale (range: 1=not severe to 7=very severe) will be used to rate the overall global severity of schizophrenia. This scale is a modification of a scale developed by the Psychopharmacology Research Branch of the National Institute of Mental Health to rate the subject's overall improvement in clinical disorder and provides a global evaluation of improvement over time from the clinician's perspective. See, e.g., Guy W. Ed. ECDEU Assessment Manual of Psychopharmacology, revised, 1976. US Department of Health, Education, and Welfare. Pub. No. (ADM), 76-338. Rockville (MD): National Institute of Mental Health-Clinical Global Impression-Improvement. p. 217-22. CGI-S will be administered and scored by the investigator or other qualified site personnel. The investigator or qualified clinician designee will rate the scale at the scheduled times. CGI-S will be administered and scored by the investigator or other qualified site personnel throughout the Double Blind Treatment Period (Days 1 to 98).

The “Positive and Negative Symptoms Scale,” “Positive and Negative Syndrome Scale,” or “PANSS” is a reliable, well known, widely used, clinician administered, validated, 30-item scale designed to evaluate the severity of various symptoms of schizophrenia and is commonly employed in clinical studies involving antipsychotics to measure symptom reduction in patients taking antipsychotics. See, e.g., European Medicines Agency [EMA], Committee for Medicinal Products for Human Use. Guidance on the clinical investigation of medicinal products, including depot preparations in the treatment of schizophrenia. 2012; EMA/CHMP/40072/2010 Rev 1; Lehman A F, Lieberman J A, Dixon L B, et al. Treatment recommendations for patients with schizophrenia. In: Practice Guideline for the Treatment of Patients with Schizophrenia. Second edition. American Psychiatric Association; 2004:3-35 (Part A); and Kay S R, Fiszbein A, Opler L A. “The positive and negative syndrome scale (PANSS) for schizophrenia.” Schizophr. Bull. 1987;13 (2): 261-76.

The subscales of the PANSS and the 5-factor model of the PANSS are commonly employed to assess different symptom domains of schizophrenia. The scale is divided into 3 sections with 7 items designed to evaluate positive symptoms (symptoms of the disease which manifest as the presence of traits), 7 items designed to evaluate negative symptoms (symptoms that manifest as the absence of traits), and 16 items that address general psychopathology. Each item is scored on a 7-point severity scale (1=absent; 2=minimal; 3=mild; 4=moderate; 5=moderate severe; 6=severe; 7=extreme). The scale also includes 3 supplementary items that constitute an aggression risk profile; however, these items will not be scored as they are not applicable to the study. The PANSS total score is derived from the summation of each item.

As used herein, the “Modified Simpson Angus Scale” or “SAS” is a clinician-administered rating scale that is widely used to assess antipsychotic-induced parkinsonism in clinical practice and research settings. See, e.g., Simpson G M, Angus J W. “A rating scale for extrapyramidal side effects.” Acta Psychiatr. Scand. Suppl. 1970; 212:11-9. The study described herein uses a modified 10-item version of the SAS (for the screening and Day 1 assessment of eligibility) in which “Leg Pendulousness” and “Head Dropping” items included in the original version have been replaced with “Head Rotation” and “Akathisia,” which has been used frequently in schizophrenia clinical trials. See, e.g., Moore T J, Furberg C D. “The harms of antipsychotic drugs: evidence from key studies.” Drug Saf. 2017;40 (1): 3-14. Each item is rated using a 5-point scale (0-4); the modified SAS scores can range from 0 to 40.

As used herein, the “Columbia-Suicide Severity Rating Scale” or “C-SSRS” is a validated, evidence-supported scale used for suicide assessment and prospectively assesses suicidal ideation and behavior. See, e.g., The World Wide Web at cssrs.columbia.edu. The C-SSRS will be administered and scored by the investigator or qualified study site personnel.

As used herein, the “EuroQol 5 Dimensions 5 Levels” or “EQ-5D-5L” is a general, single index measure for describing and valuing health. See, e.g., Herdman M, Gudex C, Lloyd A, Janssen M, Kind P, Parkin D, et al. “Development and preliminary testing of the new five-level version of EQ-5D (EQ-5D-5L).” Qual. Life Res. 2011; 20 (10): 1727-36. It defines health in terms of 5 dimensions: Mobility, Self-Care, Usual Activities, Pain/Discomfort, and Anxiety/Depression. Each dimension has 5 levels: no problems, slight problems, moderate problems, severe problems, and extreme problems. The subject indicates his/her health state by checking the box next to the most appropriate statement. The scores for the 5 dimensions can be combined into a 5-digit number that describes the patient's health state. Subjects also rate their overall health on a 0 to 100 hash-marked, vertical visual analogue scale (VAS). The endpoints are labeled “The best health you can imagine” and “The worst health you can imagine.”

The EQ-5D youth (EQ-5D-Y) version is a more comprehensible instrument suitable for children and adolescents. The EQ-5D-Y comprises 2 pages: the EQ-5D descriptive system and the EQ visual analogue scale (EQ VAS). The EQ-5D-Y descriptive system comprises the following five dimensions: mobility, looking after myself, doing usual activities, having pain or discomfort and feeling worried, sad or unhappy. Each dimension has 3 levels: no problems, some problems and a lot of problems. The younger patient is asked to indicate his/her health state by ticking the box next to the most appropriate statement in each of the five dimensions. The EQ VAS records the subjects self-rated health on a vertical visual analogue scale where the endpoints are labeled “The best health you can imagine” and “The worst health you can imagine.” The EQ-5D-5L (subjects >18 years old) and the EQ-5D-Y (subjects 13 to 17 years old) will be administered by the investigator or qualified designee.

As used herein, the “Mini International Neuropsychiatric Interview” or “MINI” is a brief structured diagnostic interview for the major psychiatric disorders (including schizophrenia) in the revised DSM Third Edition, DSM Fourth Edition, DSM-5, and International Statistical Classification of Diseases and Related Health Problems, Tenth Edition (ICD-10). See, e.g., Sheehan D V, Lecrubier Y, Sheehan K H, et al. “The Mini-International Neuropsychiatric Interview (M.I.N.I.): the development and validation of a structured diagnostic psychiatric interview for DSM-IV and ICD-10.” J. Clin. Psychiatry. 1998;59 Suppl 20:22-33; quiz 4-57. Validation and reliability studies have been done comparing the MINI to other well-known psychiatric diagnostic interviews. The results of these studies show that the MINI has similar reliability and validity properties to these instruments but can be administered in a much shorter period of time and clinicians can use it after a brief training session. See, e.g., Sheehan D V, Lecrubier Y, Sheehan K H, et al. “The validity of the Mini International Neuropsychiatric Interview (MINI) according to the SCID-P and its reliability.” Eur. Psychiatry. 1997; 12:232-41.

The assessment of the inclusion criterion for schizophrenia will be standardized using the MINI Version 7.0.2. The MINI will also be used to evaluate the presence of comorbid psychiatric disorders to assess the appropriateness of the subject for inclusion.

### Safety Endpoints

Safety endpoints include adverse events (AEs), clinical laboratory tests (hematology, clinical chemistry, and urinalysis), vital sign measurements (including orthostatic blood pressure and pulse rate), 12-lead electrocardiogram (ECG), and Columbia-Suicide Severity Rating Scale (C-SSRS).

### Other Endpoints

Other endpoints that will be evaluated include: (1) change from baseline on the Positive and Negative Syndrome Scale (PANSS) total score; (2) change from baseline on the 5-level EQ-5D version (EQ-5D-5L) Visual Analogue Scale (VAS) score; and (3) plasma concentrations of Compound (I) at Days 1, 14, 42, and 98.

1. A method of treating cognitive impairment associated with schizophrenia (CIAS) in a patient in need thereof comprising administering to the patient 1 mg to 500 mg of at least one compound chosen from Compound (I): 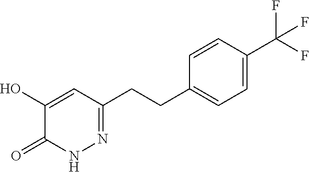 [SMILES: O=c1[nH]nc(CCc2ccc(C(F)(F)F)cc2)cc1O]
and pharmaceutically acceptable salts thereof once daily, wherein at least one cognitive symptom associated with schizophrenia in the patient is treated by the administration.

2. The method of treating cognitive impairment associated with schizophrenia according to claim 1, wherein the method comprises:
administering to the patient 1 mg to 500 mg of at least one compound chosen from Compound (I) and pharmaceutically acceptable salts thereof once daily for 6 to 8 days;
determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
continuing to administer to the patient 1 mg to 500 mg of at least one compound chosen from Compound (I) and pharmaceutically acceptable salts thereof once daily if the 6 to 8 day administration improved at least one biomarker.

3. The method of treating cognitive impairment associated with schizophrenia according to claim 1 or 2, wherein the at least one cognitive symptom associated with schizophrenia is chosen from impaired verbal memory, impaired working memory, impaired motor function, impaired attention, impaired processing speed, impaired verbal fluency, and impaired executive function.

4. The method of treating cognitive impairment associated with schizophrenia according to claim 1, wherein the administration improves the patient's Brief Assessment of Cognition in Schizophrenia (BACS) Composite Score relative to a BACS Composite Score measured for the patient prior to the administration.

5. The method of treating cognitive impairment associated with schizophrenia according to claim 1, wherein the at least one cognitive symptom associated with schizophrenia is chosen from impaired attention, impaired memory, impaired reasoning, impaired problem solving, impaired working memory, impaired processing speed, impaired language functions, and impaired social cognition.

6. The method of treating cognitive impairment associated with schizophrenia according to claim 1, wherein the administration improves the patient's Schizophrenia Cognition Rating Scale (SCORS) interviewer total score relative to a SCORS interviewer total score measured for the patient prior to the administration.

7. The method of treating cognitive impairment associated with schizophrenia according to claim 1, wherein the administration improves at least one metric chosen from:
the patient's performance on the Continuous Performance Test-Identical Pairs (CPT-IP) test relative to a CPT-IP test prior to the administration;
the patient's performance on the Brief Visuospatial Memory Test-Revised (BVMT-R) test relative to the patient's performance on a BVMT-R test prior to the administration;
the patient's performance on the Mayer-Salovey-Caruso Emotional Intelligence Test (MSCEIT) relative to the patient's performance on a MSCEIT prior to the administration;
the patient's performance on the Virtual Reality Functional Capacity Assessment Tool (VRFCAT) relative to the patient's performance on a VRFCAT prior to the administration; and
the patient's Clinical Global Impression-Severity Scale (CGI-S) score relative to a CGI-S score measured for the patient prior to the administration.

8. The method of treating cognitive impairment associated with schizophrenia according to claim 1, wherein the patient has at least one stable negative symptom associated with schizophrenia prior to the administration.

9. The method of treating cognitive impairment associated with schizophrenia according to claim 8, wherein the at least one stable negative symptom associated with schizophrenia is chosen from anhedonia, loss of motivation, and reduced interest in social interaction.

10. The method of treating cognitive impairment associated with schizophrenia according to claim 8, wherein the stability of the at least one stable negative symptom associated with schizophrenia is assessed using Positive and Negative Syndrome scale (PANSS).

11. The method of treating cognitive impairment associated with schizophrenia according to claim 8, wherein the stability of the at least one stable negative symptom associated with schizophrenia is assessed using the Brief Negative Symptom Scale (BNSS) instrument.

12. The method of treating cognitive impairment associated with schizophrenia according to claim 8, wherein the at least one stable negative symptom associated with schizophrenia is stable for at least one month prior to the administration.

13. The method of treating cognitive impairment associated with schizophrenia according to claim 1, wherein there is not statistically significant improvement in any negative symptom associated with schizophrenia in the patient following the administration.

14. The method of treating cognitive impairment associated with schizophrenia according to claim 1, wherein the at least one compound is administered in combination with at least one additional active agent.

15. The method of treating cognitive impairment associated with schizophrenia according to claim 14, wherein the at least one additional active agent treats at least one negative symptom associated with schizophrenia in the patient.

16. The method of treating cognitive impairment associated with schizophrenia according to claim 15, wherein the at least one negative symptom associated with schizophrenia is chosen from anhedonia, loss of motivation, and reduced interest in social interaction.

17. The method of treating cognitive impairment associated with schizophrenia according to claim 14, wherein the at least one additional therapeutic agent is chosen from aripiprazole, olanzapine, risperidone, and quetiapine fumarate.

18. The method of treating cognitive impairment associated with schizophrenia according to claim 1, wherein the at least one compound is administered to the patient for more than 14 weeks.

19. The method of treating cognitive impairment associated with schizophrenia according to claim 1, wherein the at least one compound is administered to the patient for more than 20 weeks.

20. The method of treating cognitive impairment associated with schizophrenia according to claim 1, wherein the patient is administered 1 mg to 100 mg of the at least one compound once daily.

21. The method of treating cognitive impairment associated with schizophrenia according to claim 1, wherein the patient is administered 20 mg or 50 mg of the at least one compound once daily.

22. The method of treating cognitive impairment associated with schizophrenia according to claim 1, wherein the patient is administered 20 mg of the at least one compound once daily.

23. The method of treating cognitive impairment associated with schizophrenia according to claim 1, wherein the patient is administered 50 mg of the at least one compound once daily.

24. The method of treating cognitive impairment associated with schizophrenia according to claim 1, wherein the administration does not treat at least one negative symptom associated with schizophrenia.

25. The method of treating cognitive impairment associated with schizophrenia according to claim 24, wherein the at least one negative symptom associated with schizophrenia is chosen from social withdrawal, affective flattening, anhedonia, diminished energy, loss of motivation, and reduced interest in social interaction.

26. The method of treating cognitive impairment associated with schizophrenia according to claim 1, wherein the patient is on a stable regimen of psychotropic medications.

27. The method of treating cognitive impairment associated with schizophrenia according to claim 1, wherein the patient was diagnosed with schizophrenia at least one year prior to the administration.

28. The method of treating cognitive impairment associated with schizophrenia according to claim 1, wherein the patient has stable symptomatology for at least 3 months prior to the administration.

29. A method of treating cognitive impairment in a patient in need thereof comprising administering to the patient a therapeutically effective amount of at least one compound chosen from Compound (I): 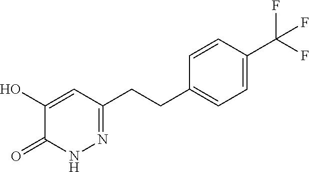 [SMILES: O=c1[nH]nc(CCc2ccc(C(F)(F)F)cc2)cc1O]
and pharmaceutically acceptable salts thereof, wherein the patient exhibits at least one cognitive symptom prior to the administration.

30. The method of treating cognitive impairment according to claim 29, wherein the method comprises:
administering to the patient a therapeutically effective amount of at least one compound chosen from Compound (I) and pharmaceutically acceptable salts thereof once daily for 6 to 8 days;
determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
continuing to administer to the patient a therapeutically effective amount of at least one compound chosen from Compound (I) and pharmaceutically acceptable salts thereof once daily if the 6 to 8 day administration improved at least one biomarker.

31. The method of treating cognitive impairment according to claim 29, wherein the at least one cognitive symptom is chosen from impaired verbal memory, impaired working memory, impaired motor function, impaired attention, impaired processing speed, impaired verbal fluency, and impaired executive function.

32. The method of treating cognitive impairment according to claim 29, wherein the at least one cognitive symptom is chosen from impaired attention, impaired memory, impaired reasoning, impaired problem solving, impaired working memory, impaired processing speed, impaired language functions, and impaired social cognition.

33. The method of treating cognitive impairment according to claim 29, wherein the administration treats the at least one cognitive symptom.

34. The method of treating cognitive impairment according to claim 29, wherein the at least one cognitive symptom is associated with a psychotic disorder.

35. The method of treating cognitive impairment according to claim 34, wherein the psychotic disorder is chosen from psychosis, schizophreniform disorder, schizoaffective disorder, and schizophrenia unassociated with aggression.

36. The method of treating cognitive impairment according to claim 34, wherein the psychotic disorder is schizophreniform disorder, schizoaffective disorder, and schizophrenia unassociated with aggression.

37. The method of treating cognitive impairment according to claim 29, wherein the psychotic disorder is schizophrenia unassociated with aggression.

38. The method of treating cognitive impairment according to claim 29, wherein the at least one cognitive symptom is associated with attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.

39. The method of treating cognitive impairment according to claim 29, wherein the at least one cognitive symptom is associated with Cushing's disease, Lewy body disease, multiple sclerosis, stroke, addictive disorder, pervasive development disorder, fragile X syndrome, Prader-Willi syndrome, and delirium.

40. The method of treating cognitive impairment according to claim 29, wherein the patient is administered 50 mg of the at least one compound once daily.

41. The method of treating cognitive impairment according to claim 29, wherein the patient is administered 20 mg of the at least one compound once daily.

42. The method of treating cognitive impairment according to claim 29, wherein the at least one compound is administered in combination with at least one additional active agent.

43. The method of treating cognitive impairment according to claim 42, wherein the patient has schizophrenia and the at least one additional active agent treats at least one negative symptom in the patient.

44. The method of treating cognitive impairment according to claim 43, wherein the at least one negative symptom is chosen from anhedonia, loss of motivation, and reduced interest in social interaction.

45. The method of treating cognitive impairment according to claim 29, wherein the patient has at least one stable negative symptom chosen from anhedonia, loss of motivation, and reduced interest in social interaction prior to the administration.

46. The method of treating cognitive impairment according to claim 1, wherein the at least one compound is administered in the form of at least one film-coated tablet.

47. The method of treating cognitive impairment according to claim 1, wherein the at least one compound is administered with water or milk.

48. A method of treating at least one cognitive symptom associated with schizophrenia in a patient, wherein the patient has no more than moderate-severe positive symptoms associated with schizophrenia (<5 rating on PANSS positive symptom items P1, P3, P4, P5, P6), comprising administering to the patient 1 mg to 500 mg of at least one compound chosen from Compound (I): 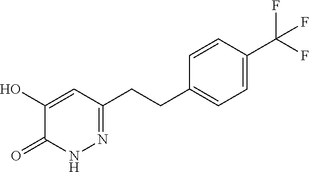 [SMILES: O=c1[nH]nc(CCc2ccc(C(F)(F)F)cc2)cc1O]
and pharmaceutically acceptable salts thereof once daily, wherein at least one cognitive symptom associated with schizophrenia in the patient is treated by the administration.

49. The method of treating at least one cognitive symptom associated with schizophrenia according to claim 48, wherein the method comprises:
administering to the patient 1 mg to 500 mg of at least one compound chosen from Compound (I) and pharmaceutically acceptable salts thereof once daily for 6 to 8 days;
determining or having determined whether the 6 to 8 day administration improved at least one biomarker chosen from the patient's eye-blink conditioning (EBC) response, the patient's mismatch negativity (MMN) amplitude, and the patient's auditory steady state response (ASSR) gamma band power; and
continuing to administer to the patient 1 mg to 500 mg of at least one compound chosen from Compound (I) and pharmaceutically acceptable salts thereof once daily if the 6 to 8 day administration improved at least one biomarker.

50. The method of treating at least one cognitive symptom associated with schizophrenia according to claim 48, wherein the administration improves the patient's Brief Assessment of Cognition in Schizophrenia (BACS) Composite Score relative to a BACS Composite Score measured for the patient prior to the administration.

51. The method of treating at least one cognitive symptom associated with schizophrenia according to claim 48, wherein the administration improves the patient's Schizophrenia Cognition Rating Scale (SCORS) interviewer total score relative to a SCORS interviewer total score measured for the patient prior to the administration.

52. The method of treating at least one cognitive symptom associated with schizophrenia according to claim 48, wherein the administration improves at least one metric chosen from:
the patient's performance on the Continuous Performance Test-Identical Pairs (CPT-IP) test relative to a CPT-IP test prior to the administration;
the patient's performance on the Brief Visuospatial Memory Test-Revised (BVMT-R) test relative to the patient's performance on a BVMT-R test prior to the administration;
the patient's performance on the Mayer-Salovey-Caruso Emotional Intelligence Test (MSCEIT) relative to the patient's performance on a MSCEIT prior to the administration;
the patient's performance on the Virtual Reality Functional Capacity Assessment Tool (VRFCAT) relative to the patient's performance on a VRFCAT prior to the administration; and
the patient's Clinical Global Impression-Severity Scale (CGI-S) score relative to a CGI-S score measured for the patient prior to the administration.

53. The method of treating at least one cognitive symptom associated with schizophrenia according to claim 48, wherein there is not statistically significant improvement in any negative symptom associated with schizophrenia in the patient following the administration.

54. The method of treating at least one cognitive symptom associated with schizophrenia according to claim 48, wherein the administration does not treat at least one negative symptom associated with schizophrenia.

55. The method of treating at least one cognitive symptom associated with schizophrenia according to claim 54, wherein the at least one negative symptom associated with schizophrenia is chosen from anhedonia, loss of motivation, and reduced interest in social interaction.

56. The method of treating at least one cognitive symptom associated with schizophrenia according to claim 48, wherein the patient is administered 50 mg of the at least one compound once daily.

57. The method of treating at least one cognitive symptom associated with schizophrenia according to claim 48, wherein the patient is administered 20 mg of the at least one compound once daily.

58. A method of increasing D-serine levels, long-term potentiation, and/or synaptic plasticity in a patient in need thereof comprising administering to the patient less than 500 mg of at least one compound chosen from Compound (I): 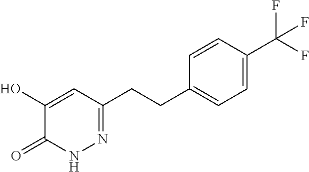 [SMILES: O=c1[nH]nc(CCc2ccc(C(F)(F)F)cc2)cc1O]
and pharmaceutically acceptable salts thereof once daily.

59. The method according to claim 58, wherein the patient is administered 50 mg of the at least one compound once daily.

60. The method according to claim 58, wherein the patient is administered 20 mg of the at least one compound once daily.

61. The method according to claim 58, wherein the method increases D-serine levels in the patient.

62. The method according to claim 58, wherein the method increases long-term potentiation in the patient.

63. The method according to claim 58, wherein the method increases synaptic plasticity in the patient.

64. The method according to claim 58, wherein the patient exhibits at least one cognitive symptom prior to the administration.

65. The method according to claim 64, wherein the at least one cognitive symptom is chosen from impaired verbal memory, impaired working memory, impaired motor function, impaired attention, impaired processing speed, impaired verbal fluency, and impaired executive function.

66. The method according to claim 64, wherein the at least one cognitive symptom is chosen from impaired attention, impaired memory, impaired reasoning, impaired problem solving, impaired working memory, impaired processing speed, impaired language functions, and impaired social cognition.

67. The method according to claim 58, wherein the administration improves at least one biomarker chosen from the patient's eye-blink conditioning (EBC) response, the patient's mismatch negativity (MMN) amplitude, and the patient's auditory steady state response (ASSR) gamma band power.

68. The method according to claim 58, wherein the patient suffers from a disease chosen from attention-deficit disorder, dementia, Alzheimer's disease, Parkinson's disease, Huntington's disease, Cushing's disease, Lewy body disease, multiple sclerosis, stroke, addictive disorder, pervasive development disorder, autism, fragile X syndrome, anxiety disorder, Prader-Willi syndrome, bipolar disorder, depression, and delirium.

69. The method according to claim 58, wherein the patient suffers from a disease chosen from psychosis, schizophreniform disorder, schizoaffective disorder, and schizophrenia unassociated with aggression.

70. The method according to claim 58, wherein the patient has no more than moderate-severe positive symptoms associated with schizophrenia (<5 rating on PANSS positive symptom items P1 (delusions), P3 (hallucinatory behavior), P4 (excitement), P5 (grandiosity), and/or P6 (suspiciousness).
